SEQUENCE LISTING (36 sequences)

SEQ ID NO: 1 - protein, 360 aa, Digenea simplex
MTVSKYNGVASTFNGVARRFDFAPLEADKLNWYPRSALPPEIPAIDLNKVNTEEELAQFLVDIRKSGLFYIVDHGIPEEISIGCYNAFREFCNLPEEAREKYNTDESFKSGGYVPFKGTSIGGGNLFERQKDFVVKFFWRGPSVVNRSPNDRFAEFHDEHHRKTAELAEKIITTILKALKTRFPEFHPDELKDNINVRNMFFSNRIYPEAPPDDGEKADYRLVPHRDLSFITLANQVPANNGFKGLFILTGDGEKIPVPPIRNSYLVFIGQGLSYLTNKYLPAALHGVDFPDNTNFEGSERASLISFYEPNDYMMPSKNINPLPEEIFEKSCTFYDDVGVGRAGTTYNYVRYKFHEGYYL

SEQ ID NO: 2 - protein, 351 aa, Palmaria palmata
MPVYNGTSQAFNFAPLHPDSLNWCPKSSLPPEIPVVDLSKLNSEAELAQFLVDIRKSGLFYVVNHGVPEELSIGCYNRFRQFCNIPEDQRAEYSTDHHFVNGGYMPYKSSSIGKANKGKSQKDFVVKYFWRGPRVENRSPDADFKSYHDEYHRRTADIANSVITKILQALTTRFPDFDPAEYKDNINSHHMFFSNRLYPDNEPEKGENVEYRLVPHRDLSFVTLAHQIPADNGYQGLFVFTGDGKKVCVPPIRNSYLVFMGQAMSYLTNKYLPAGLHGVDFPEKNCFEGSERSSLISFYEPHDHMMPSKALTPKDDEIFDRGCSFFDDIGVDKTGTTYMYVKNKFHEGYYL

SEQ ID NO: 3 - protein, 358 aa, Palmaria hecatensis
MPVYNGTPQAYNGTSQAFNFAPLDADSLTWCPESSLPPEIPAIDLSKVNSKSELAEFLVNIRKSGLFYVVNHGVPEELSMGCYNHFREFCNIPEEERAEYSTDHHFVHGGYMPYKSSSIGKANKGKDQKDFVVKYFWRGPLVENRSPSTGFKRYHDEYHRRTSDIANSVIAKIMQALTTRFPDFDPDEYKKNINSHHMFFSNRLYPDNEPEKGENAEYRLVPHRDLSFVTLAHQIPADNGYQGLFVLTGDGKKVCVPPIRNSYLVFMGQAMSYLTNKYLPAGLHGVDFPEKNCFEGSERSSLISFYEPHDHMMPSKALTPKDDEIFDRGCSFFDDIGVDKSGTAYMHVKNKFHEGYYL

SEQ ID NO: 4 - protein, 351 aa, Rhodophysema elegans
MAVYNGVSQAFDFAPLDADSLNWCPQSSLPPEIPAIDLGKVNTRAELAQFLIDIRRSGLFYVVNHGVPEELSIGCYNLFRRFCNIPEEERAKYSTDHHFVNGGYMPYKSSSIGKANMGKDQKDFVVKYFWRGPRVENRTPTAEFKKYHDEYHRRTSGVADTVIEKIMQALATRFPDFDPDEYKENTNSHHMFFSNRLYPENEPEKGENVEYRLVPHRDLSLVTLAHQIPADNGYQGLFVLTGDGKKVPVPPIRNSYLVFLGQALSYLTNKYLPAGLHGVDFPEKNSFEGSERSSLISFYEPHDRMMPSKALTPKEDEVFDRSCSFFDDIGVDTSGTTYMYVKNKFHEGYYL

SEQ ID NO: 5 - protein, 350 aa, Grateloupia filicina
MTSFDFVAKTFDFAPVKADELKWYPRSALPPEIPVIDLNNVNTEEELAQFLVDIRKSGLFYVVNHDIPEEISIQTYNEFREFCKLPEEARQKYNTDRCFFNGGYVPFKATSINGGNKNKQQRDFVVKFFWRGPHVVNRSPNERFAKWHHQYHTRTAELGEKVMTTIAKALKTRFPDFDPAELEDNVNPHNMIFSNRIYPEFPPSEGEDAEYRLTPHRDISYITLVNQTPANNGFKALFIVTGDGERVYVPPIRNSYLVFIGQSLSYLTNKYLPAALHGVAFPDGNLEGCERASLVSFYEPYDRMVPSKNIKPTAEEIAPKSCSFYDSIGCDKTGTTFTDVRAKFHSGYYV

SEQ ID NO: 6 - DNA, 1083 bp, Digenea simplex
atgacagtaagtaagtacaacggtgttgcgagtacgttcaacggtgttgcaaggagattcgactttgccccattggaggcagataaattgaattggtatcccaggtcagctcttcctccagaaatccctgctatcgacctgaataaggtcaatactgaagaagaactggctcaattcctggtcgatattcgcaaatctgggctcttttatattgttgatcatggcattccagaggagatctctataggatgttacaatgcattccgtgagttttgcaaccttcccgaggaagcaagagagaagtacaacacagatgagtcctttaaaagcggtggctatgttcctttcaaaggcacgtcgattggtggagggaacttgtttgaacgacagaaggatttcgttgtcaaattcttttggagaggaccaagtgttgtcaataggtctcctaatgatcgctttgccgaattccatgatgaacatcatcgaaagacagctgagttggccgaaaaaatcatcactaccattttgaaagccctgaagacacgctttccagaattccatcctgacgagctaaaggataatatcaatgttcgaaacatgtttttcagtaatcgaatctatccagaggctccgccagatgatggagaaaaagctgactaccgacttgttcctcatcgagatctcagttttatcactcttgcaaatcaagttccggctaacaatggattcaagggtctttttatactgacaggtgatggagaaaaaattcctgttcctccaatccggaacagctacttggtcttcatcggtcaaggcctctcatatctcacgaataagtatcttcctgcagcgcttcatggtgtggactttccagacaatacaaattttgaaggaagtgaaagagcctctctgatcagcttctacgagccaaacgattacatgatgccatccaagaacattaaccctttacctgaggagatatttgagaaaagctgtacgttttacgatgatgtcggagtagggagggctggtacaacatataattacgtgaggtacaaatttcatgaaggatactacctttaa

SEQ ID NO: 7 - DNA, 1056 bp, Rhodophysema elegans
atggcggtgtacaatggcgtctcgcaggccttcgacttcgcgccgctcgacgccgacagcctcaactggtgcccccagtcgtccctgccgcccgagatccccgccatcgacctcggcaaagtgaacaccagggccgagctcgcccagttcctgatcgacatccgcaggtccggcctcttctacgtcgtcaaccacggcgtgccggaagagctctcgatcggctgctacaacctctttcgccgcttctgcaacatccccgaggaggagcgcgccaagtacagcaccgaccatcatttcgtcaacggcggctacatgccatacaagagctcctccatcggcaaggcgaacatgggcaaggaccagaaggactttgtcgtcaagtacttctggagggggccccgcgtggagaacaggacgcccaccgccgagtttaagaagtatcacgatgagtaccatcgcaggacttcgggcgtcgcagacacggtcatagagaagattatgcaggcgctggcgacgcgctttccggacttcgaccccgacgagtacaaggagaacacgaacagccatcacatgtttttcagcaacaggctctaccccgaaaacgagccggagaagggggagaacgtcgagtaccgccttgttccgcatcgcgacctcagtttggtgacgctcgcgcaccagatccccgcagacaatggctaccagggcctgttcgttcttaccggcgacgggaagaaggtgcctgtgccgcctatccgcaacagctatctcgtcttcctggggcaggcgttgtcttatctcaccaacaagtatctgcctgctggattgcatggcgtggactttccggagaagaactcctttgaggggagcgagcggtcgtcacttatttcattctacgagccgcacgaccgcatgatgccgtcgaaggcgctcacgccaaaagaggatgaagtttttgatagaagctgctcctttttcgatgatattggtgttgatacgagtggcacgacgtatatgtacgtgaaaaataagttccacgagggctattatctatag

SEQ ID NO: 8 - DNA, 1056 bp, Palmaria hecatensis; identical to SEQ ID NO: 7

SEQ ID NO: 9 - DNA, 1056 bp, Palmaria palmata
atgcccgtgtacaacggcacctcccaggccttcaacttcgcgcccctccaccccgacagcctcaactggtgccccaagtcgtccctgccccccgagatccccgtagtcgaccttagcaaactcaactctgaggccgagctcgcccagtttctggtcgacatccgcaagtccggcctcttctacgttgtcaaccacggcgtgccggaggagctctccatcggctgctacaaccgcttccgccagttttgcaacatccccgaggaccagcgcgccgagtacagcaccgaccaccattttgtaaacggcggctacatgccctacaaaagcagttccattggcaaggcgaacaagggcaagagtcagaaggactttgtcgtcaagtacttctggagggggccccgtgttgagaacaggtcgcccgacgccgacttcaagagctatcacgacgagtaccaccggaggaccgccgacatcgcgaacagcgtcatcaccaaaatcctgcaggcgttgacaacccgcttcccagactttgaccccgctgagtacaaggacaacataaacagccaccacatgtttttcagcaatcgcctgtatcccgacaacgagccggagaaaggagagaacgtggagtaccgcctggttccgcatcgagatctcagtttcgtcaccctcgcccaccagatccccgcagacaacggctaccagggcctgttcgttttcaccggcgacgggaagaaggtctgtgtgccgcccatccgcaacagctacctcgtcttcatggggcaggcgatgtcctatctcaccaacaagtacctgcctgccggcctgcatggtgtcgactttccggagaagaactgttttgagggcagcgagcggtcgtcccttatttcgttttacgagccgcacgaccacatgatgccttcgaaggcgctgaccccgaaagatgatgaaatttttgatagaggctgctcctttttcgatgatattggtgtcgataagactggcacgacttatatgtacgtcaaaaataagttccacgagggatattatctatag

SEQ ID NO: 10 - DNA, 1053 bp, Grateloupia filicina
atgacttcgtttgatttcgttgcgaagacctttgactttgctcccgtgaaagcagatgaactgaaatggtaccccaggtctgctctccctccagaaatccctgtcatcgatctgaacaacgtcaacactgaagaagagctggctcaattcttggttgatattcgcaaatctggactcttctatgtggtcaaccatgacattccagaagagatatccatccaaacttacaacgaatttcgtgagttttgcaagctccctgaggaggccagacagaagtataacactgatagatgcttctttaatggcggttacgttcctttcaaagccacttcaattaatggaggcaacaagaacaaacagcagagagattttgtcgtaaaattcttctggagagggccgcatgtcgtcaacaggagtccgaacgagcgcttcgcaaaatggcaccaccaatatcacacaagaactgctgaactaggtgaaaaggtcatgactaccatcgcgaaagctttgaagacacgctttccggattttgaccctgctgagttggaagacaacgtcaacccgcacaacatgattttcagtaatcgaatctacccagagtttccacccagcgaaggagaggatgccgaataccgacttactcctcatagggatatcagttatatcaccctcgtgaatcaaactccggcaaacaatggctttaaggctcttttcatagtaactggtgatggggagagagtctatgttcctcccatccgcaacagttacttggtattcattggacagagcctttcatatctcaccaacaagtatcttcctgccgccctgcatggggtggcctttccagacggaaatttagaaggatgtgagagggcatctttggtttccttctacgagccttacgatcgtatggtgccttcaaaaaacatcaagcccacagcagaggagattgctccgaaaagctgttcattttacgattctatcggatgtgataagactggcacgacatttacggacgtgagggcgaaatttcattccggatactatgtttga

SEQ ID NO: 11 - protein, 439 aa, Digenea simplex
MSITTMKGVTSESPAEALSRFQTTGLTLNNPKDLYWMTEFLKEEFYDKGNYYYPIKTVCDGELIETELFCPFEPKLSPHYIQLYNSRDERSNLYAVPPKKTDMKKYNRINCEKMGSLMAPNSNYDDTEMVSLFYSMMYYLNDQTAHLKLPEEDIQPELVDELNDHVLQYLSVFLSIFKPREPQDLERIWNFLDFYQPYFKKVDGKIILHEKYQGRTPPQIGLIKKITGYVLERFAPKKNITQVIYEVIRYIKGIKQEIKIRGDKSFTLSLKEYDEFRDQVTSSPMAHSITDLTYDDFSYKAYMNPLFIKLEDLTSEIITYFNDVCTCDRERLDDDPFNSVFILRDLHSLNYVKSCDLVVKHAHDKLSKFLEIKQTLLKESTNENEKKAIAQMIKTREDSLIGYTIHEICCVTNGYARDHKPLMKDYLEKNIFKKLKATN

SEQ ID NO: 12 - protein, 488 aa, Palmaria palmata
MLPPAFLPPAVHSLFRGGLLQRPEHVGPTPTATPSAASIRAHRAPTTLVMVAHGRRRLERPTAALPTGARRTGLTLTDPADLHSMVAHLKKENFDKGNCNYPIKTVCDGELIDVQFAFPFEPKLSPHYAALYNSRDERSQLYAVPPSTTDMKKYNRINCEKMGALLAPNSAYGDTEIVSLFYSMMYYLNDQTAHFKLPEEEIQYELVDELNDNVLQYLSIFLGVFKPRDAADLERIWDFLEFYQPYFHKVDGRIELDAKYSGQTPPQVALVSKIAGYAADRFGASKNITQIIYEVIRYVKGIKEEIKIRCDKGFSLTLSEYDAFRDNVTSSPMAHSVTDLTHDAFSYEAYTNPVFNELENLTSQVITYVNDVCTCDRERLDDDPFNSVFILKNRDGLNFAEACDRVVGEVVKKTARFLETKERLLAEAADEDGRAAMAQMIKTREDSIIGYMLHEVCCVTDGYARDHKPLMKDYLEKTMFGEVASGAM

SEQ ID NO: 13 - protein, 485 aa, Palmaria hecatensis
MPPPAFLPPAVRSLFRGAPLQRLQHVGPIQSATPSAASIRARRAARTLVMVDAGPERPTAAPPTGARRTDLTLTDPADLALMVAHLKREHFDKGYCTYPIKTVCDGELIDVQFSFPFEPKLSPHYAALYNSRDERSQLYAVSPNTTDMKKYNRINCEKMGALLAPNSAYGDTEMVSLFYSMMYYLNDQTAHFKLPEEEIQYELVDELNDNVLQYLSIFLGVFEPRDAADLERIWDFLEFYQPYFNKVGGKIVLDAKYSGQTPPQVALVTKIAGYAAGRFGATKNITQIIYEVIRYVKGIKQEIKIRCDKSFTLNLAEYDAFRDQVTSSPMAHSVTDLTHDAFSYKAYTNPVFNDLENLTSQIITYVNDVCTCDRERLDDDPFNSVFILKNRDGLNFADACDLVVGEVVKKTAKFLETKDRLLAEAADEEGRAAIAQKIKTREDSIIGYMMHEVCCVTDGYARDHKPLMKEYLEKAMFGEASSGAL

SEQ ID NO: 14 - protein, 479 aa, Rhodophysema elegans
MSPSAFLPLAGPLAFRRASLQRLNHTRTLPPTVAPSRAPMTPFMVDTSRGHPATAAPPMGARRSLTLTDPADLALMVSHLKREHFDKGDYLYPIKTVCAGEDIDVRFAIPFEPRLSSHYAALYNSRNDRAKLYAVPPRTTDMRKYNRINCEKMGALLAPNSAYGDTEAVSLFYSMMYYLNDQTAICKLPEDEIELRLVDELNDNVLQYLGIFLSVFEPRDAADLERIWDFLDFYQPYFRKAGRRIVLDDKYLGQTPPQVALVTTIANYVAERFGATKNITQVVYEVIRYVKGIKQEVKIRCDKGFTLSLAEYDDFRDQVTSSPMAHSVTDLTHDAFSYEAYKNPVFNSLENLTSQVITYINDVCTCDRERLDKDPFNSVFILKDRDGLNFADACDLVVAEIEKKMAKFLETKKQLLSEAADEERRTAMAQMIKTREDSIIGYMMHEVCCVTDGYARDHKPKMKEYLEKAMFGEVSREAM

SEQ ID NO: 15 - protein, 452 aa, Grateloupia filicina
MNLILRSNMPIFPSCAFLSSSTTTTFIQPLVKRIHSVCPNPVRHEPVSANRRLVMRLAWNPTPTQQLTTESPTEALSRLQTTGLTLTDPKDLYWMTDFLEQQFYSKRNPNYPMKTICDGELIETEFHCPYQPKLSPHYMRLCNTKHERSLLYSIPPNTTDMNKYNRINCEKFASLVAPNSNYEDTEAVALMYSMMYYLNDQTAHLKLPEDMIQPQLIDELNDNVLQYLAVFLSIFEPRDPEDLERIWDFLDFYQKYFNKIDGKIVLDEKYKGPVPPQIALINKITQYISKRFAPTKNITQFIYEVIRYIKGIKQEVYIRCDKSFTLSLKEYDEFRDQVTSSPMAHAVTDMTYDNFSYKLYLNPLFTELENLTSELITYFNDVCTCDRERLDNDPFNSVFILKDLYGGTYAQSCDLVVSETRKKFSKFLEIKQILLDGAADETEKQAIAQMIK

SEQ ID NO: 16 - DNA, 1320 bp, Digenea simplex
atgagtattaccactatgaaaggagttacatcagagagtcctgccgaagcgctttcccgcttccaaactactggattgacgttgaacaaccccaaggacctttactggatgacagaatttttgaaggaagaattttacgacaagggaaactattattatcccatcaaaacggtatgtgatggcgaactcatcgaaaccgagctcttctgtcccttcgaaccaaagctcagtccacattacattcaattatacaactctcgtgatgagcgatccaacttgtacgccgttccgcccaagaaaacagacatgaaaaagtacaatcgtataaattgtgagaaaatgggttctttgatggcccctaattccaattatgacgacacagaaatggtttctctcttctactccatgatgtactacctcaatgatcagaccgctcatcttaagctccctgaagaagatattcaacctgagcttgttgatgaactcaacgaccatgtcttacagtatctaagtgtgttccttagcatctttaaaccgcgtgaacctcaagatttggagcgaatctggaattttttggacttttaccagccatatttcaagaaagttgatggcaagattatcttgcacgagaaatatcaggggcgtactccaccacaaatcggtttgatcaagaaaataacaggttatgtattagagaggtttgctccgaaaaagaatatcactcaagtcatctacgaggtcatcagatatatcaagggaatcaagcaagaaattaagattcgaggtgataagagcttcactctgtctctcaaagagtatgatgagttccgtgaccaagttacgtccagcccaatggctcattcgattacagatcttacttatgacgacttctcttacaaggcatacatgaacccactcttcatcaagctggaagatctaacctctgagatcatcacgtacttcaacgacgtttgcacatgtgatcgcgagagactggacgatgatcctttcaactctgttttcattctgagagatcttcacagtctcaactatgtgaagtcatgtgatcttgtcgttaagcatgcacatgacaagctatcaaaattcttggagatcaagcagactcttttgaaagaatctacaaacgaaaatgagaaaaaggccattgcccagatgattaagactcgtgaagattccttgattggatatacgattcatgaaatttgctgtgttacaaatgggtatgctcgtgaccacaagcccttgatgaaagattatttggagaaaaatatctttaagaaactcaaggcgacaaattaa

SEQ ID NO: 17 - DNA, 1467 bp, Palmaria palmata
atgcttcctcctgccttcctccctccggccgtgcactccctcttccgcggcggcctgctgcagcgtccggaacacgttggtccaaccccaactgcaacgccttccgccgcttcgatccgcgcacaccgcgcaccaacgacgctcgtcatggtcgcgcacggtcggcgccgtctcgaacgccccacagcggcactaccaaccggcgcgcgacggaccggcttgacgctcaccgacccggccgacctgcattccatggtggcgcacctgaaaaaggagaacttcgacaagggcaactgcaactacccaatcaagacggtgtgcgatggagaactcatcgacgtccagttcgccttcccgtttgaaccgaagctgagcccgcactacgccgccctgtacaattcgcgtgacgagcgctcccagctgtacgctgtcccgcctagtactacagatatgaaaaaatacaaccgcatcaactgcgagaagatgggcgccctgctcgcgccgaactctgcgtatggcgacacggagatagtgtcgctgttctattcgatgatgtattatctcaatgaccagacggcgcacttcaagctaccagaggaagagatccagtacgagctcgtggacgagctcaacgacaatgtgctgcagtatctcagcatatttctgggcgttttcaagccgcgcgatgctgcggatttggagcgcatttgggactttctcgagttttaccagccctattttcacaaggtcgacgggaggattgagttagatgccaagtatagcgggcagacgccgccgcaggtcgcgcttgtctcgaagattgcggggtacgccgcggatcggttcggcgcgtcaaagaacatcacccagatcatctacgaggtcattcgatatgtcaagggcatcaaggaggagatcaagatcaggtgcgacaagggctttagcctcaccctctccgagtacgacgcgttccgggacaatgtcacatcgagcccgatggcgcactccgtaaccgacctgacgcacgatgcgttctcctacgaggcctacacgaaccccgtcttcaacgagctggaaaacctgacctcccaagtcatcacctacgtcaacgacgtgtgtacgtgcgaccgggagcggctggacgacgacccgttcaattcggtctttattctgaaaaatcgcgacggtctgaacttcgccgaagcgtgcgatcgcgttgtcggcgaggtggtgaagaagacggcaaggttcctggagacgaaggagcggcttcttgcggaggctgctgatgaagacgggagggcggccatggcgcagatgatcaagacgcgcgaagactcgatcatcgggtacatgctgcacgaggtctgctgcgtgacggatgggtatgcgcgcgaccacaagccgttgatgaaagactatctggagaagacgatgtttggtgaggtggccagcggagcaatgtag

SEQ ID NO: 18 - DNA, 1458 bp, Palmaria hecatensis
atgcctcctcccgccttcctccctccagccgtgcgctccctcttccgcggcgccccgctgcagcgtctgcaacacgtcggtccaatccaaagtgcaacgccctccgctgcttcgatccgcgcacgccgtgcagcaaggacgctcgtcatggtcgacgcaggcccggagcgccccacagcagcaccaccaaccggcgcgcgacggaccgacttgacgctaacggacccggcagatctggcgctcatggtcgcccatctgaaaagggagcacttcgacaaggggtactgcacctacccgataaagacggtgtgcgatggagaactcatcgacgtccagttctccttcccgttcgagccgaagctgagcccgcactacgcggccctgtacaattcgcgtgacgagcgctcgcagctgtacgctgtctcgccaaatactacggatatgaagaagtacaaccgcatcaactgcgagaagatgggcgcgctgcttgccccgaactctgcatatggcgacacggagatggtttcgctgttctactcgatgatgtattatctcaatgaccagacggcgcacttcaagctgcccgaggaggagatccagtacgagcttgtggacgagctcaacgacaatgtgctgcagtatctcagcatttttctgggcgtgttcgagccgcgcgatgctgcggacttggagcgtatttgggactttctcgagttttaccagccgtattttaacaaggtcggcgggaagatcgtgctggatgccaagtacagcgggcagacgccgccgcaggtcgcgctcgtcacgaagatcgcggggtacgccgcggggcgcttcggcgcgacgaagaacatcacgcagatcatctacgaggtcattcgatatgttaagggcatcaagcaggagatcaagatcaggtgcgacaagagctttaccctcaacctcgcggagtacgacgcgttccgcgaccaggtcacatcgagcccgatggcgcactctgtaaccgacttgacgcacgacgccttctcctacaaggcctacacgaaccccgtcttcaacgacctggaaaacctgacctcccaaatcatcacctatgtcaacgacgtatgcacttgtgaccgcgagcggctggacgacgatccgttcaactcggtctttattctgaaaaatcgcgacggcctcaacttcgccgacgcgtgcgacctcgttgtcggcgaggtcgtaaagaagacggcgaagttcctggagactaaggataggcttcttgcggaagctgctgatgaagaggggagggcggctattgcgcagaagatcaagacgcgcgaggactcgatcatcgggtacatgatgcacgaggtttgctgcgtgacggacgggtatgcgcgcgaccacaagccgctgatgaaagagtatctggagaaggcgatgtttggtgaggcgagcagcggagccctgtag

SEQ ID NO: 19 - DNA, 1440 bp, Rhodophysema elegans
atgtctccttcggcctttctccccctagccgggcccttagccttccggcgggcctcgctgcagcgtctaaatcatacccgtaccctcccacccaccgtcgctccgtcccgcgcaccaatgacgcccttcatggtcgacaccagccgcgggcaccctgctacagcagccccacctatgggcgcgcgccgcagcctgacgctgacggacccggccgatctggcgctcatggtctcccacctgaagcgcgagcatttcgacaagggcgactacctttacccgatcaaaaccgtatgcgccggcgaggacatcgacgtccgcttcgctatccccttcgagccgcgcctcagctcgcactatgcggccctgtacaactcgcgaaacgatcgcgccaagctttacgccgtcccgccgcggacgactgacatgcgtaagtacaaccgcatcaactgtgagaagatgggcgccctgctcgcgcccaactctgcctacggcgacacagaggccgtctctctcttctactcgatgatgtactacctcaacgaccagacggcgatctgcaagctgcccgaggacgaaatcgagcttcgcctcgtcgacgagctcaacgacaacgtgctccaatatctcggcatcttcctctccgtcttcgagccgcgcgatgccgctgatttggagcgtatctgggactttctagacttttaccagccgtacttccgcaaggccggccggaggatcgtgctggacgacaaatacctcgggcagacgccgccgcaggtcgcgcttgtcacaacgatcgcaaactacgtcgccgaacgcttcggcgcgacgaagaacatcacgcaggtcgtctacgaggtgattcgctatgttaagggcatcaagcaggaggtgaagatcaggtgcgacaagggattcaccctctcactcgccgagtacgacgatttccgcgaccaggtcacgtccagcccgatggcgcactccgtgaccgacctcacccacgacgccttctcctacgaggcatataagaacccggtcttcaactccctggagaacctgacctcccaggtcattacctacatcaacgacgtgtgcacgtgtgaccgagagcggctagacaaggacccgtttaactcggtgttcattctgaaagaccgcgacggtctgaacttcgccgacgcgtgcgacctcgtcgtcgccgagatcgaaaagaaaatggcaaagttcctcgagacgaagaagcagcttctgtcggaggctgctgatgaagagcgcagaacagccatggcgcagatgatcaagacgcgggaggactcgatcatcggctacatgatgcacgaggtctgctgcgtgacggacgggtatgcgcgcgaccacaagccgaagatgaaggagtatctggagaaggcgatgtttggggaggtgagcagggaagccatgtag

SEQ ID NO: 20 - DNA, 1356 bp, Grateloupia filicina
atgaatttgatcctccgcagtaacatgcctattttcccatcttgcgcttttctctcatcctcaaccactaccactttcattcaacctcttgtcaaacgtatccactccgtttgccccaatccagtacgccatgagccagtttcagcaaaccgtcgcctagtcatgcgacttgcctggaatcctacacccactcaacaacttaccacagagagtcctaccgaagcactttcccgccttcaaaccactggactaaccctgactgacccgaaggacctttactggatgaccgattttttggaacaacaattttactcaaaaagaaaccccaactatcccatgaaaaccatctgcgacggcgaactcatcgagaccgaatttcactgtccataccaacccaaactcagccctcattatatgaggttgtgcaacactaagcatgaaagatctctcttgtactcaattccaccaaacaccacagatatgaacaagtacaaccgaattaattgcgagaagttcgcctctttggtagccccgaattccaattatgaagatacagaggctgttgccctcatgtactccatgatgtactatctcaatgatcagactgcccatcttaaacttcctgaggatatgattcagcctcaacttattgatgaactcaacgataacgtattgcagtatctcgctgtgtttctaagcatcttcgaaccccgcgatccggaagatttggagcgcatctgggacttcttagacttttatcagaagtacttcaacaaaatagacggaaagattgtcttggatgaaaagtacaaaggaccagtcccgcctcaaattgctttgattaacaaaatcacccaatatatctcgaagagatttgctccgacgaagaacataactcagttcatctacgaggtcatcagatacatcaaggggatcaagcaagaggtctacattcgatgtgataagagttttactctttctctcaaggagtatgatgaatttcgtgatcaagtgacgtccagcccgatggcacatgctgtgacagatatgacctatgacaacttctcttataagctctacctgaacccgcttttcaccgaactggagaatctgacttcagaactgatcacctacttcaacgacgtttgcacatgtgatcgcgagagactggacaatgatcctttcaactctgtgttcattctgaaagacctttatggtggtacctatgcacaaagttgtgatcttgtcgtgagcgaaacgcgcaaaaagttctctaaatttttggagatcaaacaaattcttcttgatggggcagcagacgaaaccgagaaacaagctattgcccagatgattaag

SEQ ID NO: 21 - DNA, 51 bp, Artificial sequence
  Synthetic polynucleotide
ggtgccgcgcggcagccatatgagtattaccactatgaaaggagttacatc

SEQ ID NO: 22 - DNA, 47 bp, Artificial sequence
  Synthetic polynucleotide
ggtggtggtggtgctcgagttaatttgtcgccttgagtttcttaaag

SEQ ID NO: 23 - DNA, 47 bp, Artificial sequence
  Synthetic polynucleotide
ggtgccgcgcggcagccatatgacagtaagtaagtacaacggtgttg

SEQ ID NO: 24 - DNA, 47 bp, Artificial sequence
  Synthetic polynucleotide
ggtggtggtggtgctcgagttaaaggtagtatccttcatgaaatttg

SEQ ID NO: 25 - DNA, 54 bp, Artificial sequence
  Synthetic polynucleotide
ctgtacttccaatccggatccatgacttctttcgatttcgtcgctaagactttc

SEQ ID NO: 26 - DNA, 49 bp, Artificial sequence
  Synthetic polynucleotide
ggtggtggtggtgctcgagttagacgtagtaccccgagtgaaactttgc

SEQ ID NO: 27 - DNA, 50 bp, Artificial sequence
  Synthetic polynucleotide
ctgtacttccaatccggatccatgcctgtgtataatggtacgtcacaagc

SEQ ID NO: 28 - DNA, 53 bp, Artificial sequence
  Synthetic polynucleotide
ggtggtggtggtgctcgagttacaggtagtaaccttcatggaatttgttcttg

SEQ ID NO: 29 - DNA, 49 bp, Artificial sequence
  Synthetic polynucleotide
ctgtacttccaatccggatccatggcggtgtacaatggcgtctcgcagg

SEQ ID NO: 30 - DNA, 56 bp, Artificial sequence
  Synthetic polynucleotide
ggtggtggtggtgctcgagctatagataatagccctcgtggaacttatttttcacg

SEQ ID NO: 31 - DNA, 22 bp, Artificial sequence
  Synthetic polynucleotide
catatggctgccgcgcggcacc

SEQ ID NO: 32 - DNA, 28 bp, Artificial sequence
  Synthetic polynucleotide
ctcgagcaccaccaccaccaccactgag

SEQ ID NO: 33 - DNA, 1057 bp, Artificial sequence
  Synthetic polynucleotide
atgcctgtgtataatggtacgtcacaagcattcaattttgcgccgctgcacccagatagtttgaattggtgcccaaagtcgagtcttcctccagaaattcctgtagtggatctgtcaaaacttaattctgaagccgagctggcacaatttcttgtggacattcgtaagagcgggttgttttatgtagtcaatcatggtgtcccggaagaactttctattgggtgttacaatcgcttccgtcaattctgtaacattcctgaggaccaacgtgcggaatactcgaccgaccatcattttgtaaacgggggctacatgccgtataagagttccagcatcggcaaggcaaataaaggtaaatctcaaaaggactttgtggttaaatacttctggcgtggaccgcgcgttgagaaccgtagtccggatgctgatttcaagagctatcacgacgaataccaccgtcgcacagctgatatcgctaacagcgtgattactaaaattcttcaggccctgacgacgcgttttcctgactttgaccctgctgaatataaagataacattaattcccatcatatgttcttctcgaaccgcctttacccagacaacgaacctgagaaaggcgagaatgtagaataccgtttagtaccgcaccgtgacctgagcttcgtgacccttgctcaccaaatcccggcggacaacggttaccaaggccttttcgtttttaccggcgatgggaagaaggtttgcgtgcctccgattcgtaacagctatcttgtcttcatgggacaggcaatgtcgtatctgacgaataagtacttacctgcagggttgcatggcgtggattttcctgaaaagaactgtttcgaggggtctgaacgctcctctcttatctcattttacgaacctcacgatcatatgatgcccagcaaagcacttacacctaaggatgatgagatcttcgatcgtggttgttcgttctttgatgacatcggcgtggacaaaacagggacgacctatatgtatgtcaagaacaaattccatgaaggttactacctgtaag

SEQ ID NO: 34 - DNA, 1053 bp, Artificial sequence
  Synthetic polynucleotide
atgacttctttcgatttcgtcgctaagactttcgattttgctcccgtcaaagctgacgagttgaagtggtatccacgttccgcgttaccaccagaaattcctgtaattgatttgaacaacgtcaatacggaggaggagcttgcacagttcctggtggacatccgcaagtctggacttttctacgttgtcaaccacgatatcccggaggaaatctcaatccagacgtacaacgaatttcgtgagttttgtaaattgccagaagaggcccgccaaaaatacaatactgatcgctgcttcttcaatgggggatatgttccattcaaagccacgagcattaatgggggtaataagaacaaacagcaacgcgactttgtcgtgaagtttttctggcgcggcccacacgtggtcaatcgttcccccaatgaacgctttgccaaatggcaccatcagtatcatacacgcactgctgaattaggtgaaaaggtcatgaccacaatcgcaaaagctttgaagacgcgctttcctgacttcgacccggccgaactggaggacaatgtgaaccctcacaacatgattttttctaaccgcatttatccggaattcccacccagtgaaggggaggatgctgagtatcgcctgaccccccaccgtgatatttcctacattacccttgtgaatcagactcctgccaataacggattcaaagcattatttatcgtaaccggggacggcgaacgcgtttacgtccctcctatccgcaactcttacttggtattcatcgggcagagcttgagctaccttaccaataaataccttccagcggcacttcacggggtcgcttttccggacggcaatcttgagggttgcgagcgtgcctcactggtgtctttctatgaaccgtatgatcgcatggtcccatcgaaaaatatcaaaccaacggcagaagagatcgctccaaaatcctgctccttctatgattcgattgggtgtgacaagacaggaactacctttactgatgttcgtgcaaagtttcactcggggtactacgtctaa

SEQ ID NO: 35 - protein, 482 aa, Pseudo Nitzschia multiseries
MKFATSIVAAIATTGAAFTVIPQKLSHPSQLNALNTMGSISSITAESPKEVLSRVQDAGLTLTNPNDLYWMVDFLKEKYYDNGDYYYPIKTVCDGESIDVKFYCPFEPSLSPHYLELYGSRDERASIYETTMKKYNRINSEKTSAICTPYSSYGDTQIVAYFYSMMYYINDQTAHLKLPESEIESELIDILNDDILIYLNEFMSIFEPEDAQDLERIWDFLDFYQPYFSKVDGKIVLDEKYLVRTPSQMPLIKTICEYVSEQFAPSKNITQVIWEVVRYIKGVKDEIHIRGDKSFTLSLQEYDDFRDKVTASPMAHAVSDLTHERFSYEAYTNPAFMELENRCSEIITYFNDVCTSDRERLDEDPFNSVFILMDLDPSLNFAKSCDVVVEHAYNKMQAFLKLKEEILESASDEEERLALARMIKTREDSLIGYVLHEVCCVEDGYARDHKPLMKAFLEEEITKSLAEKVKFNPVESESVRLN

SEQ ID NO: 36 - protein, 373 aa, Pseudo Nitzschia multiseries
MTVAINNETVVLTPNEDDVQVNKGKTLETSFPPLKGDDLKWFPRSSLPAEIPAIDIGKVSTKEELEQFLVDIRKSGLFYIVNHGVPEEVSINVYNAFREFISTTTEEERMKYYTDTHFQNGGYVPFQGSSIRGGNLGKPQKDHVVKYFWRGPEVINRTPNEKFTEAHNMHHTETFKVAEKVIRTIFKALKLRFPDFDPMEFENTINSKKMFFTNRIYPQAEPSDEEEITHRLVPHLDTSFITLANQVPADNGFQGLFVETGDGKKVKVPGIRNSYLVFIGQSLSYLTKNYLPSALHGVDKPPSDLFEGSERSSLITFYEPAEIIIPSKNINPNPEETSESCPFFYDIGLTVNDPEGTTWDFVKNKFITGYYAD

(19) United States
(12) Patent Application Publication
(10) Pub. No.: US 2024/0279695 A1
(43) Pub. Date: Aug. 22, 2024

(54) SCALABLE BIOSYNTHESIS OF THE SEAWEED NEUROCHEMICAL KAINIC ACID

(71) Applicant: The Regents of the University of California, Oakland, CA (US)

(72) Inventors: Bradley S. Moore, La Jolla, CA (US); Jonathan R. Chekan, La Jolla, CA (US); Shaun M. K. McKinnie, La Jolla, CA (US)

(21) Appl. No.: 17/298,464

(22) PCT Filed: Dec. 3, 2019
(86) PCT No.: PCT/US2019/064221

§ 371 Date: May 28, 2021

Related U.S. Application Data
(60) Provisional application No. 62/775,327, filed on Dec. 4, 2018.

(51) Int. Cl. C12P 17/18; C12N 9/02; C12N 9/10

(52) U.S. Cl. CPC C12P 17/188; C12N 9/0071; C12N 9/1085; C12Y 114/11; C12Y 205/01

(57) ABSTRACT

Provided herein are compounds of Formulas (la), (lb), (II) to (VIII), and salts thereof, and methods of making the same. Also provided herein are recombinant proteins useful in the production of compounds disclosed herein, polynucleotides encoding the same, and cells comprising the same.

### RELATED PATENT APPLICATION

This patent application claims the benefit of U.S. Provisional Patent Application No. 62/775,327 filed on Dec. 4, 2018. The entire content of the foregoing patent application is incorporated herein by reference, including all text, tables and drawings.

### STATEMENT OF GOVERNMENT SUPPORT

This invention was made with government support under National Institute of Health/National Institute of General Medical Services (NIH/NIGMS) Grant No. R01 GM085770. The government has certain rights in this invention.

### BACKGROUND

Kainic acid, the flagship member of the kainoid family of natural neurochemicals, is a widely used neuropharmacology agent that helped unravel the key role of ionotropic glutamate receptors, including the kainate receptor, in the central nervous system. Worldwide shortages of this seaweed natural product in 2000 prompted numerous chemical syntheses that now number in excess of 70, including scalable preparations with as few as six-steps. Recently, the biosynthesis of the kainoid domoic acid was established in Pseudo-nitzschia diatoms that revealed nature's molecular logic in efficiently constructing their shared pyrrolidine ring with three contiguous stereogenic centers.

### SUMMARY

Improved methods for the production of kainic acid and kainic acid lactone are needed. In embodiments, methods and compositions of the present disclosure address this need, and provide additional advantages as well.

A two-enzyme biosynthetic pathway to kainic acid was discovered in red macro algae. The biosynthetic genes appear to be co-clustered in genomes of Digenea simplex and the edible Palmaria palmata. A series of scalable biosynthetic strategies were explored to efficiently generate kainic acid on a gram scale which strategies included enzyme total synthesis, fermentation in a heterologous host, chemoenzymatic synthesis, and biotransformation.

### BRIEF DESCRIPTION OF THE DRAWINGS

FIG. 1 shows A) domoic acid and B) kainic acid biosynthetic pathways, in accordance with some embodiments. An illustrative kainic acid biosynthesis (kab) gene cluster from C) D. simplex and D) P. palmata are also shown. Scale bar is representative for both (C) and (D).

FIG. 2 shows (A) High Molecular Weight (HMW) DNA was sequenced on the PromethION platform producing 47 Gb of long read sequencing data with an N50 read length of 7 kb. (B) Nanopore sequence has an average quality score of 10. (C) Zoomed in view of assembly graph color coded by GC % reveals D. simplex genome contigs (red), bacterial contigs (blue), and low, unclassified contigs (green). D. simplex genome contigs are clustered in a hairball due to the repeat structures in this genome. One bacterium appeared as a 6 Mb assembly. (D) GC % by length plot reveals D. simplex genome and bacterial genomes.

FIG. 3 shows LCMS analysis of DsKabA and DsKabC in vitro assays. Each trace represents the (M-H)⁻ extracted ion chromatogram for prekainic acid (214.1:0.5 m/z) and kainic acid (212.1:0.5 m/z). DsKabA converts L-Glu and DMAPP to prekainic acid in a Mg²⁺-dependent manner (purple traces). DsKabC can convert prekainic acid (PKA) to kainic acid (KA) and kainic acid lactone (KA Lactone), but turnover is nearly undetectable when the iron chelator EDTA is added (cyan traces).

FIG. 4 shows that the discovery of the kainic acid biosynthetic genes enabled new kainic acid production routes: a) two-enzyme biosynthesis, b) one-enzyme chemoenzymatic synthesis, c) bacterial fermentation, and d) biotransformation. Lyophilized kainic acid in a scintillation vial is shown in e). Bold arrows highlight a strategy to produce large scale amounts of kainic acid.

FIG. 5 shows amino acid sequence alignment of the domoic acid DabA and kainic acid KabA proteins. The accession numbers for proteins from this study can be found in Table S3 and the Pseudo-nitzschia multiseries DabA NCBI accession number is AYD91073.1.

FIG. 6 shows amino acid sequence alignment of the domoic acid DabC and kainic acid KabC proteins. The accession numbers for proteins from this study can be found in Table S3 and the Pseudo-nitzschia multiseries DabC NCBI accession number is AYD91075.1.

FIG. 7 shows 12% SDS-PAGE loaded with EZ-Run Rec Protein Ladder (Fisher Bioreagents), DsKabA, and DsKabC.

FIG. 8 shows that DsKabA substrate specificity was analyzed by LCMS. Each trace represents the extracted ion chromatogram for the expected (M-H)⁻ product mass±0.5 m/z. DMAPP+L-Glu: 214.1 m/z, DMAPP+D-Glu: 214.1 m/z, DMAPP+L-Gln: 213.1 m/z, DMAPP+L-Asp: 200.1 m/z, DMAPP+L-Asn: 199.1 m/z, DMAPP+Gly: 142.2 m/z, and GPP+L-Glu: 282.2 m/z.

FIG. 9 shows that an aqueous D. simplex extract was analyzed by LCMS and compared to synthetic/enzymatic standards to verify the presence of kainic acid, kainic acid lactone, and prekainic acid. Each trace represents the (M-H)⁻ extracted ion chromatogram for prekainic acid (214.1±0.1 m/z) and kainic acid/kainic acid lactone (212.1±0.1 m/z). The D. simplex trace extracted for the prekainic acid mass (214.1±0.1 m/z) was magnified tenfold.

FIG. 10 shows the reaction products of KabC enzymes from D. simplex, P. palmata, R. elegans, and G. filicinia were analyzed by LCMS following a 5 h incubation. DsKabC was used in the His6 tag cleaved form while the other three homologs were tested as an MBP-fusion. Each trace represents the (M-H)⁻ extracted ion chromatogram for both prekainic acid (214.1±0.5 m/z) and kainic acid/kainic acid lactone (212.1±0.5 m/z).

FIG. 11 shows kainic acid production by in vitro conversion of synthetic prekainic acid by DsKabC. The trace represents the (M-H)⁻ extraction ion chromatogram for prekainic acid (PKA, 214.1±0.1 m/z) and kainic acid/kainic acid lactone (KA/KA Lactone, 212.1±0.1 m/z). The prekainic acid peak is <0.3% of the kainic acid intensity and is not visible.

FIG. 12 shows a time course of the conversion of prekainic acid to kainic acid in the media of E. coli expressing the dskabC gene on a pET28 vector. Each trace represents the (M-H)⁻ extracted ion chromatogram for both prekainic acid (214.1±0.1 m/z) and kainic acid (212.1±0.1 m/z).

FIG. 13 shows results illustrating the purity of kainic acid isolated from biotransformation by E. coli, as tested by ¹H NMR.

FIG. 14 presents data that relates to NMR analysis of kainic acid and kainic acid lactone.

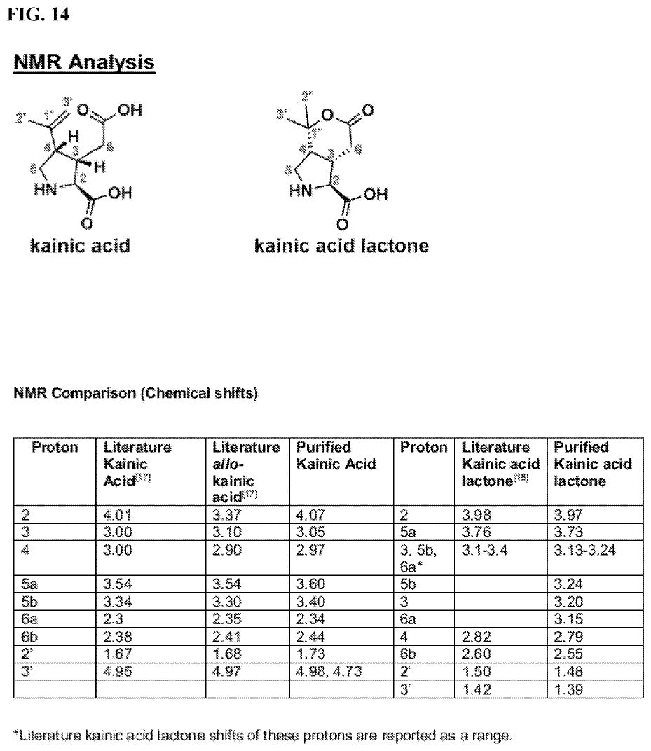

FIG. 15 shows NMR correlations for prekainic acid, kainic acid, and kainic acid lactone.

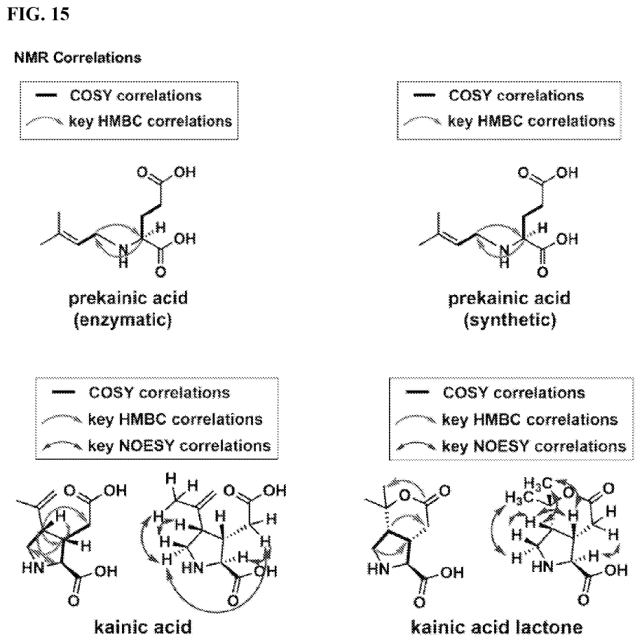

FIG. 16 shows ¹H NMR analysis of prekainic acid (DsKabA enzymatic product).

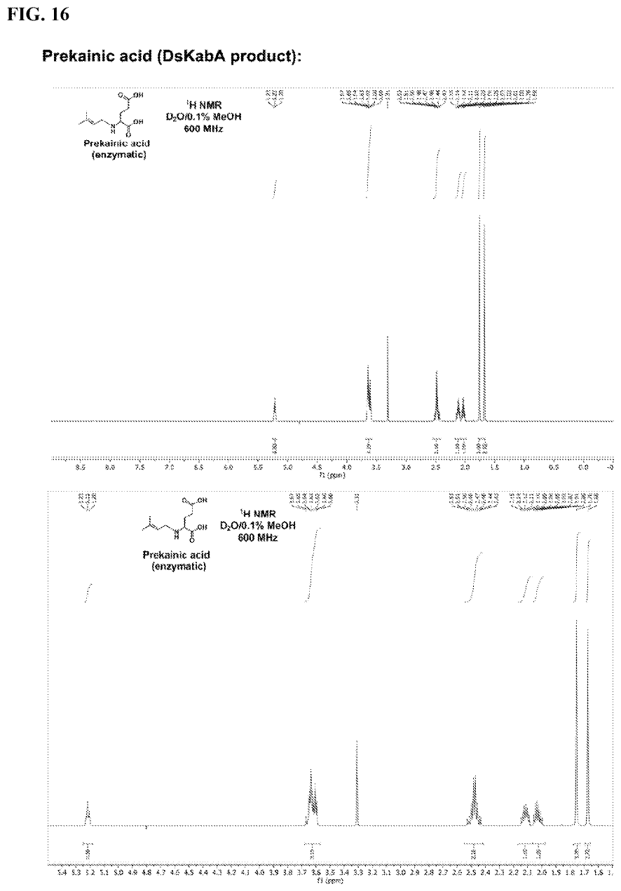

FIG. 17 shows COSY and HSQC analysis of prekainic acid (enzymatic product).

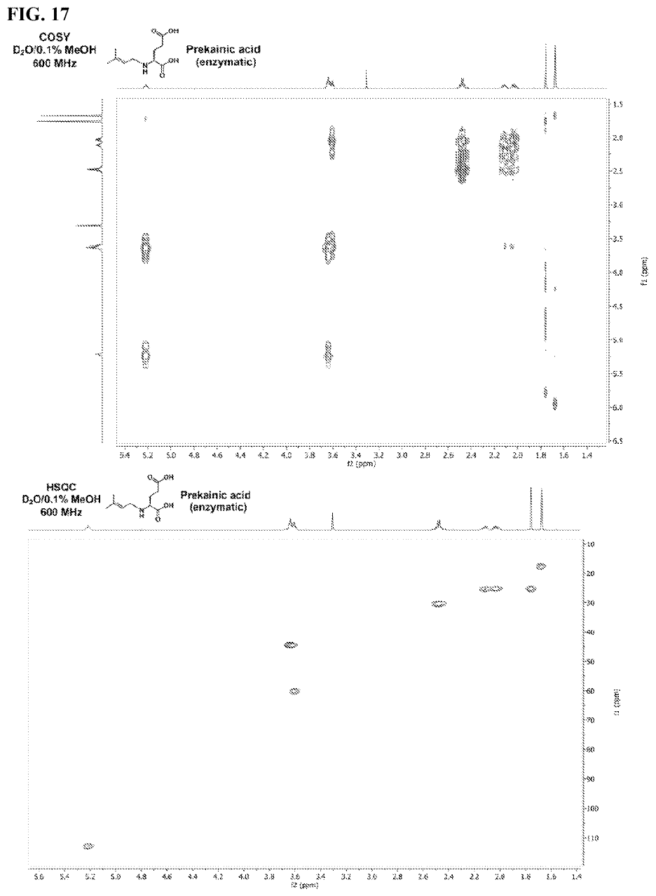

FIG. 18 shows HMBC and NOESY analysis of prekainic acid (enzymatic product).

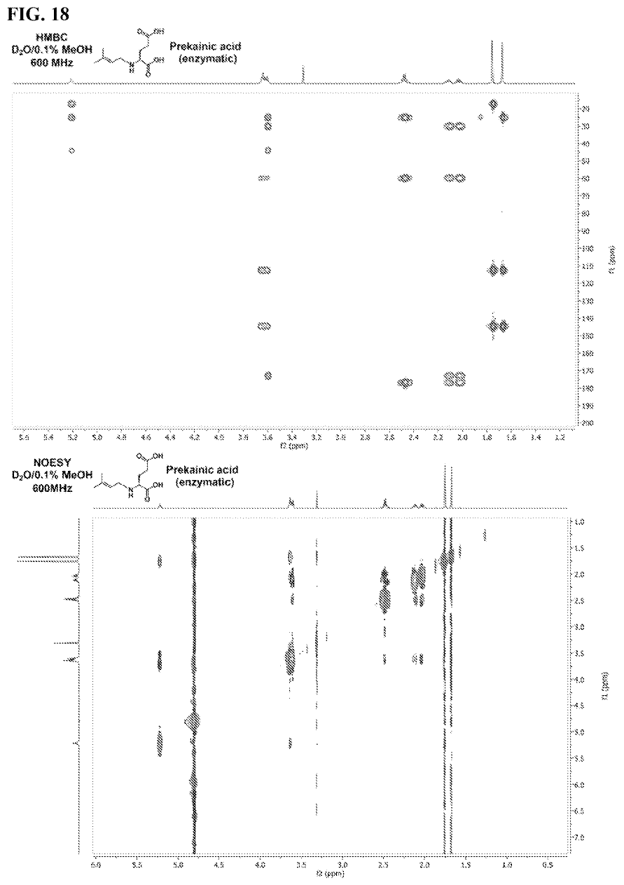

FIG. 19 shows ¹H NMR analysis of prekainic acid (synthetic product).

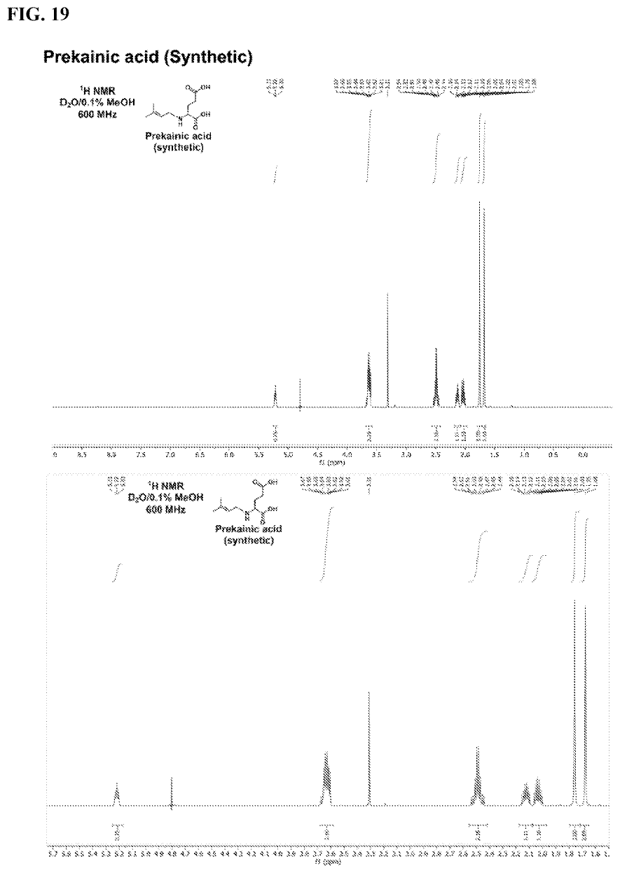

FIG. 20 shows COSY and HSQC analysis of prekainic acid (synthetic product).

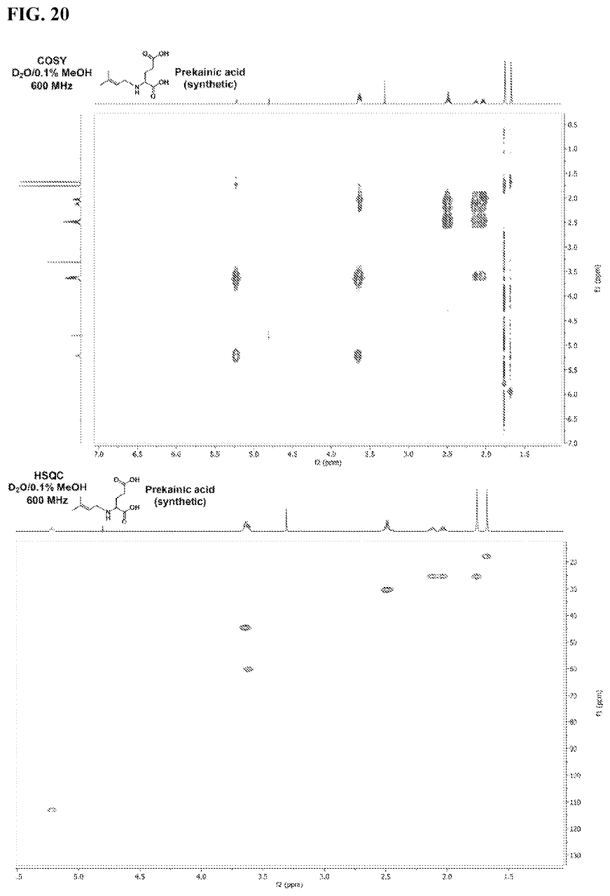

FIG. 21 shows HMBC and NOESY analysis of prekainic acid (synthetic product).

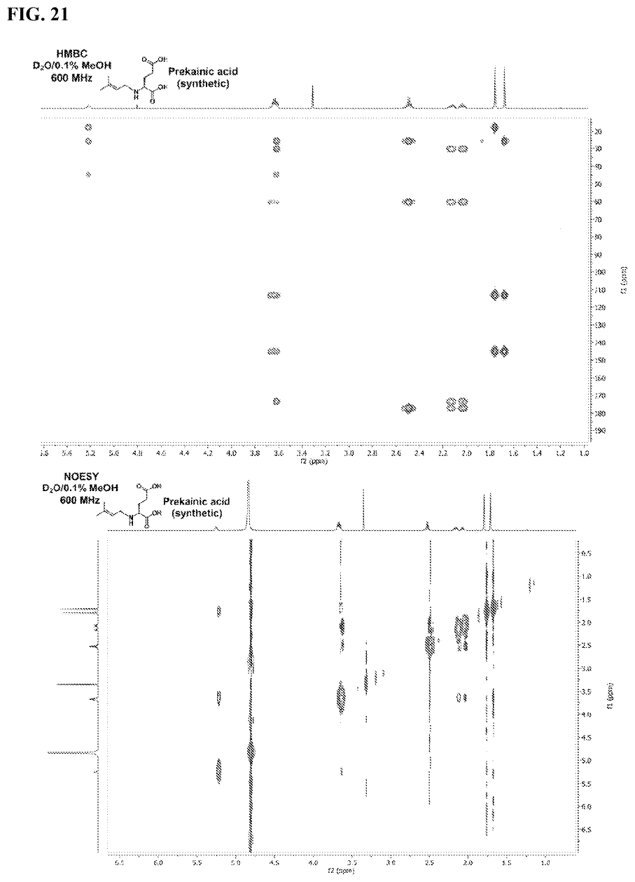

FIG. 22 shows ¹H NMR analysis of kainic acid (DsKabC or enzymatic product).

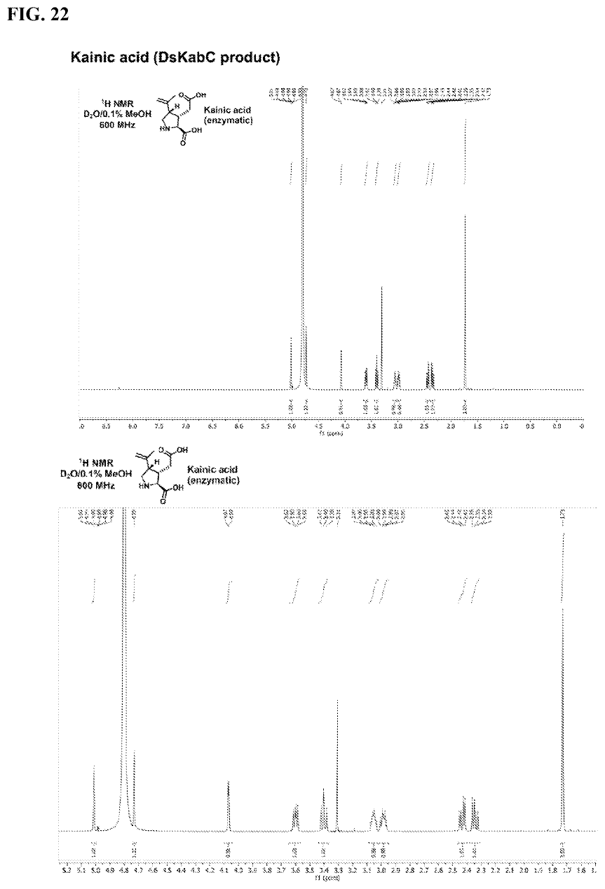

FIG. 23 shows COSY and HSQC analysis of kainic acid (DsKabC enzymatic product).

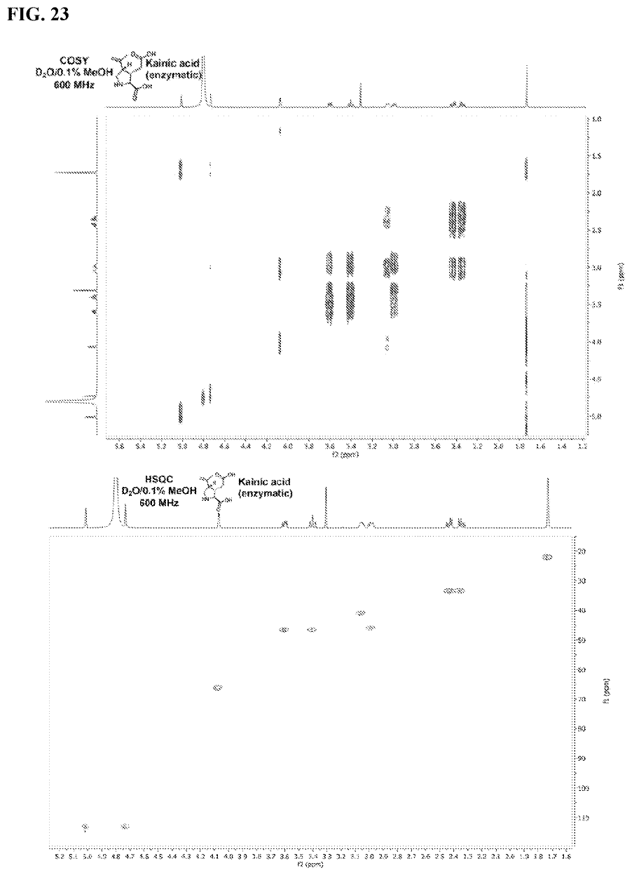

FIG. 24 shows HMBC and NOESY analysis of prekainic acid (DsKabC or enzymatic product).

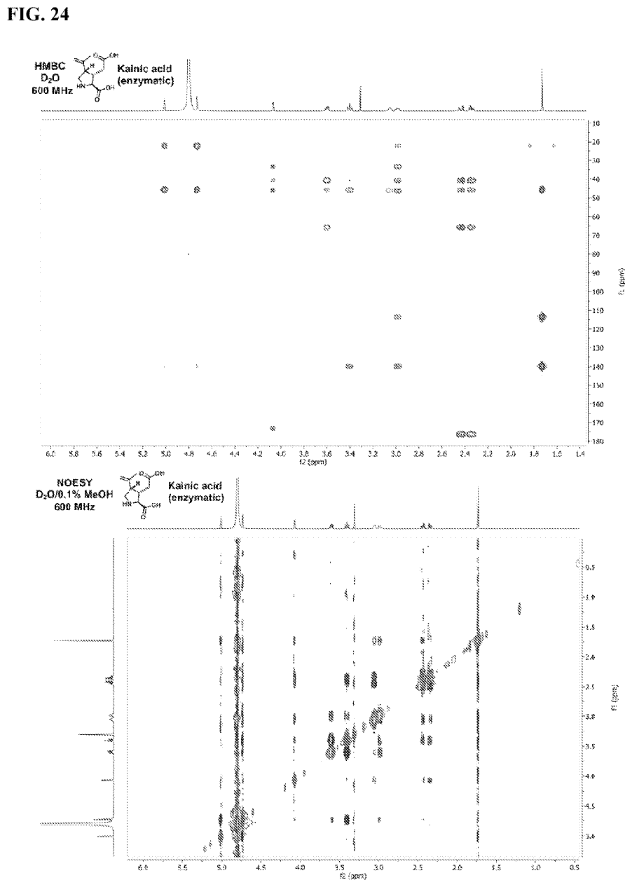

FIG. 25 shows ¹H NMR analysis of kainic acid lactone (GfKabC enzymatic product).

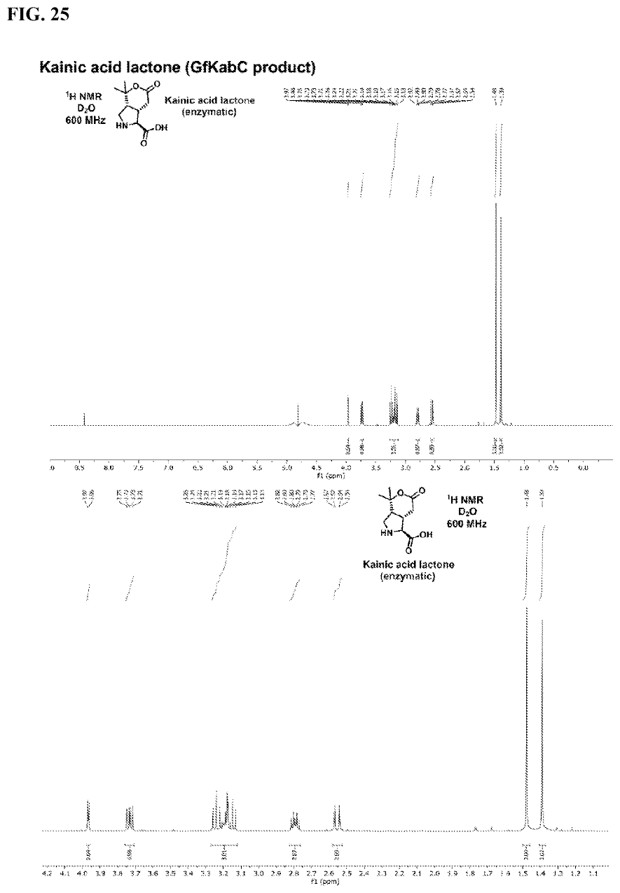

FIG. 26 shows DEPT ¹³C NMR and COSY analysis of kainic acid lactone (enzymatic product).

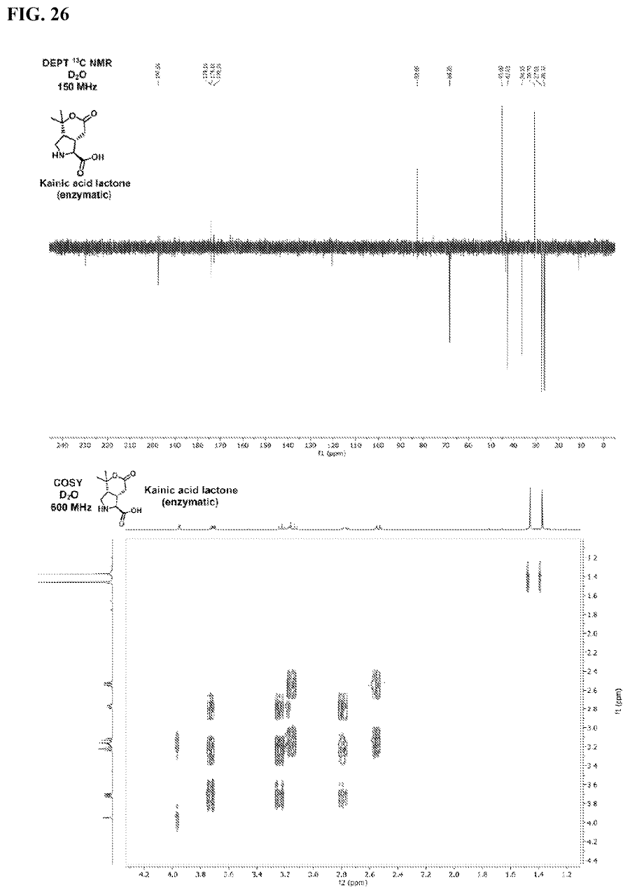

FIG. 27 shows HSQC and HMBC analysis of kainic acid lactone (enzymatic product).

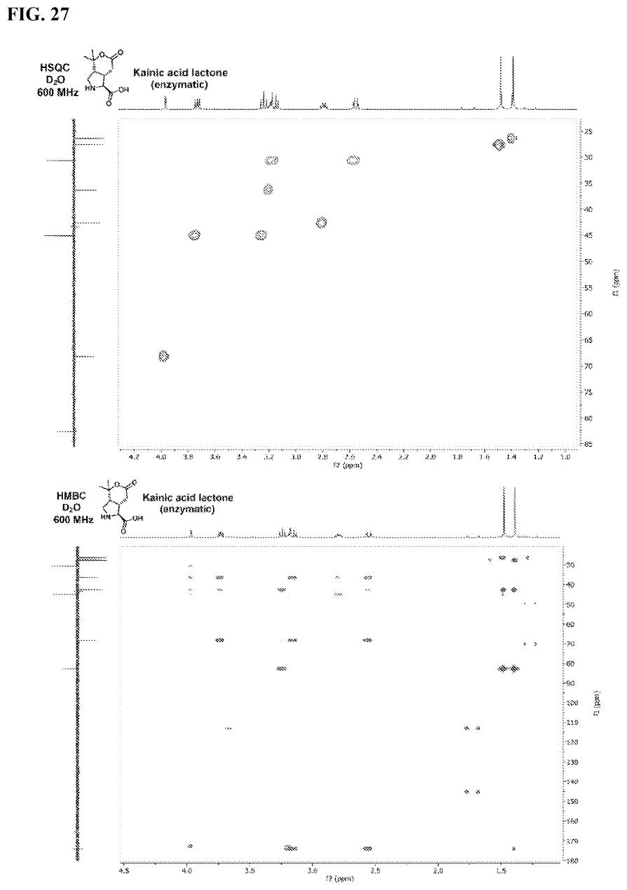

FIG. 28 shows NOESY analysis of kainic acid lactone (enzymatic product).

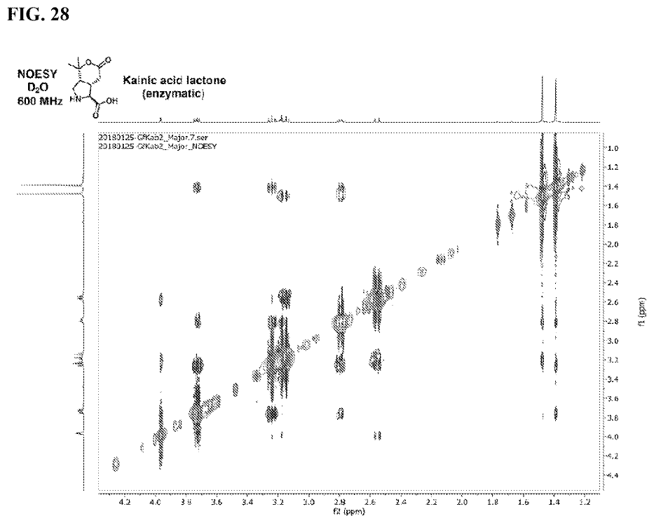

FIG. 29 shows analysis of kainic acid production by a coupled in vitro reaction of DsKabA and DsKabC as monitored by LCMS after an 18 h incubation. The trace represents the (M-H)⁻ extracted ion chromatogram for prekainic acid (PKA, 214.1±0.1 m/z) and kainic acid/kainic acid lactone (KA/KA Lactone, 212.1±0.1 m/z).

FIG. 30 shows LCMS analyses of lysed cell pellets/M9 media of E. coli expressing different DsKab gene combinations. Samples were tested for the presence of both prekainic acid (PKA) and kainic acid (KA). Each trace represents the (M-H)⁻ extracted ion chromatogram for prekainic acid (214.1±0.1 m/z) and kainic acid (212.1±0.1 m/z).

### DETAILED DESCRIPTION OF THE INVENTION

Provided herein, inter alia, are processes for preparation of kainic acid and kainic acid lactone. In one aspect, provided herein is a process for the preparation of a compound with the formula,

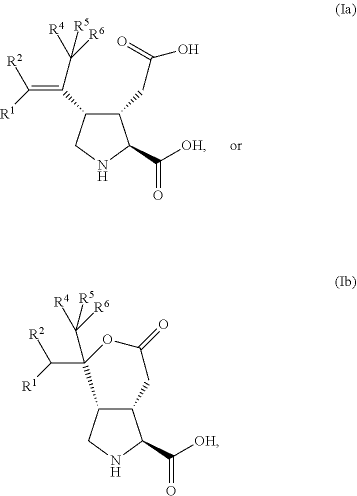

or a salt thereof.

The compound or a salt of Formula (Ia) or (Ib) is prepared by cyclizing a compound of formula II.

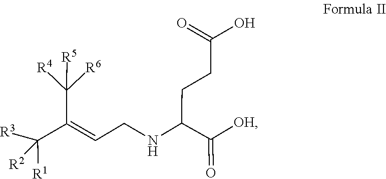

or a salt thereof.

In Formula II, (a) at least one of R¹, R², R³, R⁴, R⁵ or R⁶ is hydrogen, and
(b) R¹ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R² is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R³ is hydrogen, halogen, —CN, —C(O)OH, —CONH₂, —NO₂, —SO₂Cl, —SO₂NH₂, —NHNH₂, —NHSO₂CH₃, —N₃, —NR^{3A}R^{3B}, —OR^{3A}, —SR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R⁴ is hydrogen, halogen, —CN, —C(O)OH, —CONH₂, —NO₂, —SO₂Cl, —SO₂NH₂, —NHNH₂, —NHSO₂CH₃, —N₃, —NR^{4A}R^{4B}, —OR^{4A}, —SR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R⁵ is hydrogen, halogen, —CN, —C(O)OH, —CONH₂, —NO₂, —SO₂Cl, —SO₂NH₂, —NHNH₂, —NHSO₂CH₃, —N₃, —NR^{5A}R^{5B}, —OR^{5A}, —SR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R⁶ is hydrogen, halogen, —CN, —C(O)OH, —CONH₂, —NO₂, —SO₂Cl, —SO₂NH₂, —NHNH₂, —NHSO₂CH₃, —N₃, —NR^{6A}R^{6B}, —OR^{6A}, —SR^{6A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. any two of R¹, R² or R³ substituents or any two of R⁴, R⁵ or R⁶ substituents may optionally be joined to form a substituted or unsubstituted aryl or substituted or unsubstituted heteroaryl. any one of R¹, R² or R³ and any one of R⁴, R⁵ or R⁶ may optionally be joined to form a substituted or unsubstituted cycloalkyl, substituted or unsubstituted aryl or substituted or unsubstituted heteroaryl.
each R^{1A}, R^{2A}, R^{3A}, R^{4A}, R^{5A}, R^{6A}, R^{1B}, R^{2B}, R^{3B}, R^{4B}, R^{5B}, and R^{6B} is independently a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

Cyclization of compound of Formula II is accomplished by contacting compound of Formula II with a recombinant KabC polypeptide.

In another aspect, provided herein is a process for the preparation of a compound with the formula,

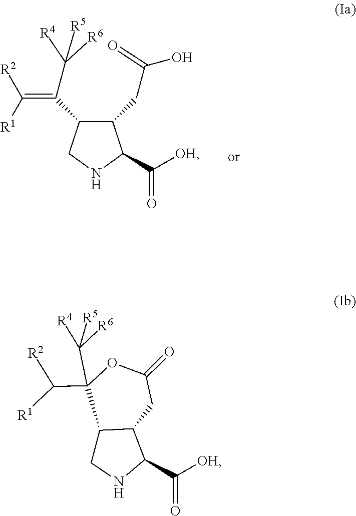

or a salt thereof.

The compound or a salt of Formula (Ia) or (Ib) is prepared by contacting a compound of Formula III:

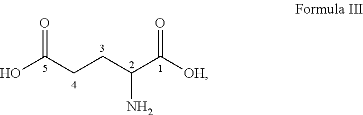

or a salt thereof, with a compound of Formula V:

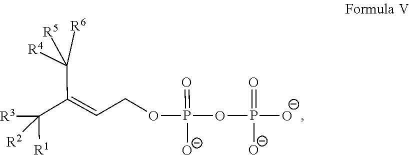

or a salt thereof, a recombinant KabA polypeptide, a recombinant KabC polypeptide, and a α-keto-glutarate.

In an aspect, provided herein is a compound of formula II:

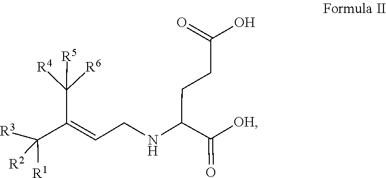

or a salt thereof.

In an aspect, provided herein is a compound of Formula VI:

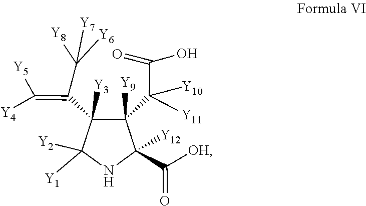

or a salt thereof. In Formula VI, one of Y₁, Y₂, Y₃, Y₄, Y₅, Y₆, Y₇, Y₈, Y₉, Y₁₀, Y₁₁, or Y₁₂ is a hydrogen that is isotopically enriched with deuterium or tritium, and the rest of Y₁, Y₂, Y₃, Y₄, Y₅, Y₆, Y₇, Y₈, Y₉, Y₁₀, Y₁₁, and Y₁₂ are non-enriched hydrogen atoms.

In an aspect, provided herein is a compound of Formula VII:

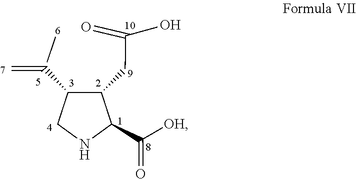

or a salt thereof. In Formula VII, 1, 2, 3, 4, 5, 6, 7, 8, 9, and 10 are carbon atoms. At least one of 1, 2, 3, 4, 5, 6, 7, 8, 9, or 10 carbon atoms is isotopically enriched with carbon-13 or carbon-14.

In an aspect, provided herein is a compound of Formula VIII:

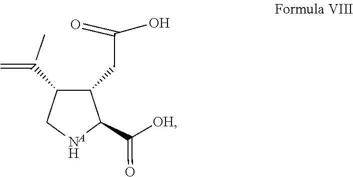

or a salt thereof. In Formula VIII, N^{A} is a nitrogen atom which is isotopically enriched with nitrogen-15.

In an aspect, provided herein is a recombinant polynucleotide encoding a KabA polypeptide, a KabC polypeptide, or both.

In an aspect, provided herein is an expression vector comprising the recombinant polynucleotide.

In an aspect, provided herein is a recombinant cell which includes a polynucleotide or an expression vector.

In an aspect, provided herein is a process for the preparation of a compound with the formula,

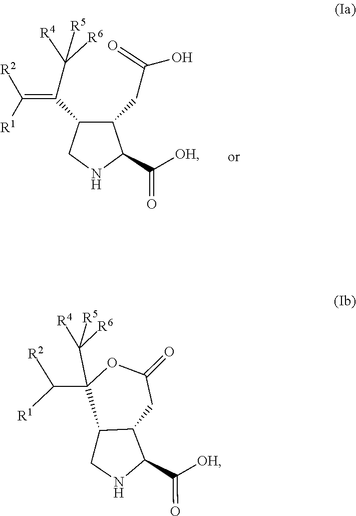

or a salt thereof.

In embodiments, the compound or a salt of Formula (Ia) or (Ib) is prepared by culturing a recombinant cell in a culture medium.

### DETAILED DESCRIPTION

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature. Methods, devices and materials similar or equivalent to those described herein can be used in the practice of this invention.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein or enzyme.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the TUPAC-TUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

As used herein, the terms “analogue” and “analog”, in reference to a chemical compound, refers to a compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

As used herein, the term “modified nucleotide” is used in accordance with its plain and ordinary meaning and refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both.

As used herein, the term “selective” or “selectivity” or the like of a compound refers to the compound's ability to discriminate between molecular targets.

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The terms “KabA protein” and “KabA polypeptide” refer to a protein (including homologs, isoforms, and functional fragments thereof). The term includes any recombinant or naturally-occurring form of a KabA polypeptide or variants thereof that maintain KabA polypeptide activity (e.g. within at least 30%, 40%, 50%, 60%, 70%, 80%, 90%, 95%, or 100% activity compared to wildtype KabA). In embodiments, a KabA polypeptide encoded by a kabA gene has an amino acid sequence set forth in any one of SEQ ID NOs:11-15. In embodiments, the amino acid sequence or nucleic acid sequence is the sequence known at the time of filing of the present application.

The terms “KabC protein” and “KabC polypeptide” refer to a protein (including homologs, isoforms, and functional fragments thereof). The term includes any recombinant or naturally-occurring form of a KabC polypeptide or variants thereof that maintain KabC polypeptide activity (e.g. within at least 30%, 40%, 50%, 60%, 70%, 80%, 90%, 95%, or 100% activity compared to wildtype KabC). In embodiments, a KabC polypeptide encoded by a kabC gene has an amino acid sequence set forth in any one of SEQ ID NOs:1-5. In embodiments, the kabC gene comprises a nucleic acid sequence set forth in any one of SEQ ID NOs:6-10. In embodiments, the amino acid sequence or nucleic acid sequence is the sequence known at the time of filing of the present application.

The terms “KabA gene” and “KabA polynucleotide” as used herein refer to the any of the recombinant or naturally-occurring forms of a kabA gene or variants or homologs thereof that code for a KabA polypeptide capable of maintaining the activity of a KabA polypeptide (e.g., within at least 50%, 80%, 90%, 95%, 96%, 97%, 98%, 99% or 100% activity compared to an endogenous KabA polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring kabA gene. In some embodiments, a KabA polynucleotide comprises a nucleotide sequence having at least 90%, 95%, 96%, 97%, 98%, 99% or 100% nucleic acid sequence identity to NCBI Accession Numbers QCC62379, QCC62384, QCC62371, QCC62373, QCC62377, or a portion thereof. In some embodiments, a KabA polynucleotide comprises a nucleotide sequence having at least 90%, 95%, 96%, 97%, 98%, 99% or 100% nucleic acid sequence identity to any one of SEQ ID NOs:16-20.

The terms “KabC gene” and “KabC polynucleotide” as used herein refer to the any of the recombinant or naturally-occurring forms of a kabC gene or variants or homologs thereof that code for a KabC polypeptide capable of maintaining the activity of a KabC polypeptide (e.g., within at least 50%, 80%, 90%, 95%, 96%, 97%, 98%, 99% or 100% activity compared to an endogenous KabC polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring kabC gene. In some embodiments, a KabC polynucleotide comprises a nucleotide sequence having at least 90%, 95%, 96%, 97%, 98%, 99% or 100% nucleic acid sequence identity to NCBI Accession Numbers QCC62383, QCC62385, QCC62372, QCC62374, QCC62378, or a portion thereof. In some embodiments, a KabC polynucleotide comprises a nucleotide sequence having at least 90%, 95%, 96%, 97%, 98%, 99% or 100% nucleic acid sequence identity to SEQ ID NO:33 or 34. In some embodiments, a KabC polynucleotide comprises a nucleotide sequence having at least 90%, 95%, 96%, 97%, 98%, 99% or 100% nucleic acid sequence identity to any one of SEQ ID NOs:6-10.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

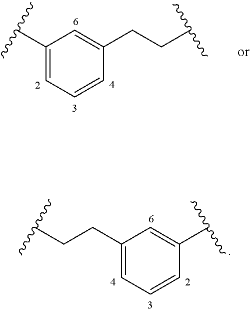 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from/zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from/zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or“lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accor and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

The term “non-nucleophilic base” as used herein refers to any sterically hindered base that is a poor nucleophile.

The term “nucleophile” as used herein refers to a chemical species that donates an electron pair to an electrophile to form a chemical bond in relation to a reaction. All molecules or ions with a free pair of electrons or at least one pi bond can act as nucleophiles.

The term “strong acid” as used herein refers to an acid that is completely dissociated or ionized in an aqueous solution. Examples of common strong acids include hydrochloric acid (HCl), nitric acid (HNO₃), sulfuric acid (H₂SO₄), hydrobromic acid (HBr), hydroiodic acid (HI), perchloric acid (HClO₄), or chloric acid (HClO₃).

The term “carbocation stabilizing solvent” as used herein refers to any polar protic solvent capable of forming dipole-dipole interactions with a carbocation, thereby stabilizing the carbocation.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Kainic acid, the flagship member of the kainoid family of natural neurochemicals, is a widely used neuropharmacology agent that helped unravel the key role of ionotropic glutamate receptors, including the kainate receptor, in the central nervous system. Worldwide shortages of this seaweed natural product in 2000 prompted numerous chemical syntheses that now number in excess of 70, including scalable preparations with as few as six-steps. Recently, the biosynthesis of the kainoid domoic acid was established in Pseudo-nitzschia diatoms that revealed nature's molecular logic in efficiently constructing their shared pyrrolidine ring with three contiguous stereogenic centers.

The tropical seaweed Digenea simplex has been used for centuries in Asia as an antihelmenthic agent to treat parasitic worm infections¹. In the 1950s, the active compound, kainic acid, was isolated^{2,3}, enabling its use as a combination treatment for Ascaris infections up until the 1990s⁴-6. While the exact antihelmenthic mechanism of action remains unclear, kainic acid functions as an ionotropic glutamate receptor (iGluR) agonist⁷. iGluRs mediate neuronal cell-cell commutation by binding to glutamate and facilitating influx of Ca²⁺ into the cell⁸. Structurally similar to glutamic acid, kainic acid can bind more efficiently to iGluRs and thereby stimulate excessive influx of Ca²⁺, which leads to excitotoxicity and cell death9. In fact, kainic acid was important in the initial discovery and characterization of several classes of iGluRs, particularly the eponymous kainate receptor¹⁰. The iGluR agonistic bioactivity is shared with the other members of the kainoid natural product family, namely domoic acid¹¹ and acromelic acid¹². Notably, domoic acid is a major environmental toxin produced primarily by oceanic diatom blooms^{13,14} and causes severe neurological problems when consumed through contaminated seafood^{15,16}. While kainic acid does not appear to produce excitotoxic effects for humans in vivo, its bioactivity has been exploited to create mouse model systems to study neurological diseases17, particularly temporal lobe epilepsy^{18,19}.

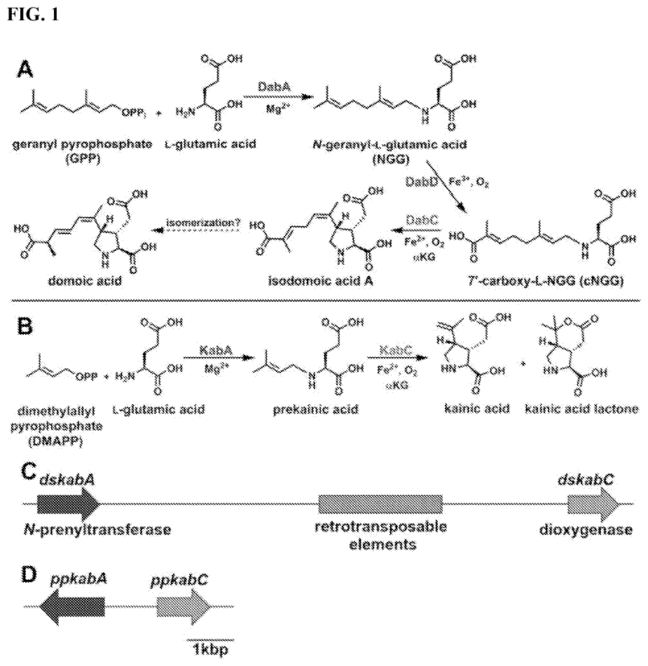

Even though kainic acid was discovered more than 50 years ago and over 70 synthetic routes have been established²⁰, there has been little progress made on elucidating how it is constructed by seaweeds. In fact, although red macroalgae are prolific producers of natural products, with over 700 compounds identified, only five genomes are currently available, which has curtailed biosynthetic discovery science in algae through modern genome mining approaches²¹. Recently, we established the biosynthetic logic for domoic acid production in microalgal Pseudo-nitzschia multiseries diatoms through the discovery of a four-gene cassette (dabA-D) and the confirmation of their in vitro enzymatic functions²². The structural similarity between domoic acid and kainic acid allowed us to propose a conserved route of biosynthesis (FIG. 1A). N-prenylation of L-glutamate with dimethylallyl pyrophosphate (DMAPP) via a DabA homolog would produce a “prekainic acid” pathway intermediate that could conceivably be directly cyclized with a DabC homolog to generate kainic acid. Using a genomics approach, we identified red macroalgal kainic acid gene clusters and leveraged these newly identified enzymes to develop in vitro and in vivo kainic acid production routes. Together, our results demonstrate the advantages of new sequencing technologies to discover biosynthesis genes from challenging eukaryotic genomes and further reinforce the utility of enzymes in the manufacturing of fine chemicals.

In one aspect, provided herein is a process for the preparation of a compound with the formula,

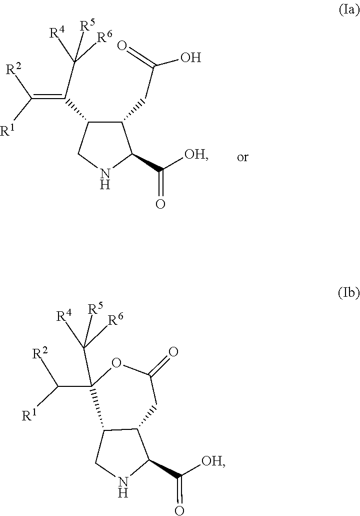

or a salt thereof.

The compound or a salt of Formula (Ia) or (Ib) is prepared by cyclizing a compound of formula II.

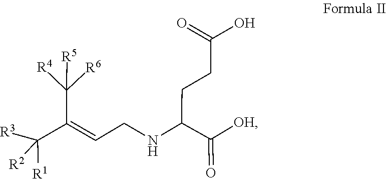

or a salt thereof.

In Formula II, (a) at least one of R¹, R², R³, R⁴, R⁵ or R⁶ is hydrogen, and
(b) R¹ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
   R² is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
   R³ is hydrogen, halogen, —CN, —C(O)OH, —CONH₂, —NO₂, —SO₂Cl, —SO₂NH₂, —NHNH₂, —NHSO₂CH₃, —N₃, —NR^{3A}R^{3B}, —OR^{3A}, —SR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
   R⁴ is hydrogen, halogen, —CN, —C(O)OH, —CONH₂, —NO₂, —SO₂Cl, —SO₂NH₂, —NHNH₂, —NHSO₂CH₃, —N₃, —NR^{4A}R^{4B}, —OR^{4A}, —SR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
   R⁵ is hydrogen, halogen, —CN, —C(O)OH, —CONH₂, —NO₂, —SO₂Cl, —SO₂NH₂, —NHNH₂, —NHSO₂CH₃, —N₃, —NR^{5A}R^{5B}, —OR^{5A}, —SR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
   R⁶ is hydrogen, halogen, —CN, —C(O)OH, —CONH₂, —NO₂, —SO₂Cl, —SO₂NH₂, —NHNH₂, —NHSO₂CH₃, —N₃, —NR^{6A}R^{6B}, —OR^{6A}, —SR^{6A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹ and R² may optionally be joined to form an oxo. Any two of R¹, R² or R³ substituents or any two of R⁴, R⁵ or R⁶ substituents may optionally be joined to form a substituted or unsubstituted aryl or substituted or unsubstituted heteroaryl. Any one of R¹, R² or R³ and any one of R⁴, R⁵ or R⁶ may optionally be joined to form a substituted or unsubstituted cycloalkyl, substituted or unsubstituted aryl or substituted or unsubstituted heteroaryl. Each R^{1A}, R^{2A}, R^{3A}, R^{4A}, R^{5A}, R^{6A}, R^{1B}, R^{2B}, R^{3B}, R^{4B}, R^{5B}, and R^{6B} is independently a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, the cyclization of compound of Formula II is accomplished by contacting compound of Formula II with a recombinant KabC polypeptide. In embodiments, the cyclization of compound of Formula II is accomplished by contacting compound of Formula II with a purified KabC polypeptide and a α-keto-glutarate.

In embodiments, the process for the preparation of compound having the formula (Ia) or (Ib) includes the incubation of D. simplex KabC (DsKabC), compound of Formula II (prekainic acid), α-keto-glutarate, L-ascorbate and ferrous iron.

Particular process conditions, including one or more of reaction time, temperature, solvent, reagent, amount of reagent(s) order of reagent addition, pH etc. may be selected in order to optimize reaction product purity and/or yield.

In embodiments, ferrous iron is ferrous hydroxide, ferrous halide, ferrous nitrate, ferrous sulfate, ferrous oxide, ferrous carbonate, ferrous acetate, ferrous chromate. In embodiments, ferrous iron is ferrous sulfate.

In embodiments, the incubation is carried out in a buffer at pH of about 5.0, about 5.5, about 6.0, about 6.5, about 7.0, about 7.5, about 8.0, about 8.5, about 9.0, about 9.5, or about 10.0. In embodiments, the incubation is carried out in a buffer of 300 mM potassium chloride and 50 mM HEPES pH 8.0.

In embodiments, the incubation is carried out at about 5° C., about 10° C., about 15° C., about 20° C., about 25° C., about 30° C., about 35° C., about 40° C., or about 45° C. In embodiments, the incubation is carried out at about 22° C. In embodiments, the incubation is carried out for about 5 hours, about 10 hours, about 15 hours, about 20 hours, about 25 hours, about 30 hours, about 35 hours, about 40 hours, or about 45 hours. In embodiments, the incubation is carried out for about 18 hours.

In embodiments, (a) at least one of R¹, R², R³, R⁴, R⁵ or R⁶ in Formula II is a hydrogen, and (b) R¹ is hydrogen, substituted or unsubstituted C₁-C₁₀ alkyl, substituted or unsubstituted C₅-C₁₀ aryl, or substituted or unsubstituted 5 to 10 membered heteroaryl. R² is hydrogen, substituted or unsubstituted C₁-C₁₀ alkyl, substituted or unsubstituted C₅-C₁₀ aryl, or substituted or unsubstituted 5 to 10 membered heteroaryl. R₃ is hydrogen, substituted or unsubstituted C₁-C₁₀ alkyl, substituted or unsubstituted C₅-C₁₀ aryl, or substituted or unsubstituted 5 to 10 membered heteroaryl. R⁴ is hydrogen, substituted or unsubstituted C₁-C₁₀ alkyl, substituted or unsubstituted C₅-C₁₀ aryl, or substituted or unsubstituted 5 to 10 membered heteroaryl. R⁵ is hydrogen, substituted or unsubstituted C₁-C₁₀ alkyl, substituted or unsubstituted C₅-C₁₀ aryl, or substituted or unsubstituted 5 to 10 membered heteroaryl. R⁶ is hydrogen, substituted or unsubstituted C₁-C₁₀ alkyl, substituted or unsubstituted C₅-C₁₀ aryl, or substituted or unsubstituted 5 to 10 membered heteroaryl. any two of R¹, R² or R³ substituents or any two of R⁴, R⁵ or R⁶ substituents may optionally be joined to form a substituted or unsubstituted C₅-C₁₀ aryl or substituted or unsubstituted 5 to 10 membered heteroaryl. Any one of R¹, R² or R³ and any one of R⁴, R⁵ or R⁶ may optionally be joined to form a substituted or unsubstituted C₃-C₈ cycloalkyl, substituted or unsubstituted C₅-C₁₀ aryl or substituted or unsubstituted 5 to 10 membered heteroaryl.

In embodiments, (a) at least one of R¹, R², R³, R⁴, R⁵ or R⁶ in Formula II is a hydrogen, and (b) R¹ is hydrogen or substituted or unsubstituted C₁-C₁₀ alkyl. R² is hydrogen or substituted or unsubstituted C₁-C₁₀ alkyl. R³ is hydrogen or substituted or unsubstituted C₁-C₁₀ alkyl. R⁴ is hydrogen or substituted or unsubstituted C₁-C₁₀ alkyl. R⁵ is hydrogen or substituted or unsubstituted C₁-C₁₀ alkyl. R⁶ is hydrogen or substituted or unsubstituted C₁-C₁₀ alkyl. any two of R¹, R² or R³ substituents or any two of R⁴, R⁵ or R⁶ substituents may optionally be joined to form a substituted or unsubstituted C₅-C₁₀ aryl or substituted or unsubstituted 5 to 10 membered heteroaryl. Any one of R¹, R² or R³ and any one of R⁴, R⁵ or R⁶ may optionally be joined to form a substituted or unsubstituted C₃-C₈ cycloalkyl, substituted or unsubstituted C₅-C₁₀ aryl or substituted or unsubstituted 5 to 10 membered heteroaryl.

In embodiments, R¹, R², R³, R⁴, R⁵ and R⁶ are hydrogen.

In embodiments, R¹ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, where R¹ is substituted, R¹ is substituted with a substituent group. In embodiments, where R¹ is substituted, R¹ is substituted with a size-limited substituent group. In embodiments, where R¹ is substituted, R¹ is substituted with a lower substituent group.

In embodiments, R^{1A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{1A} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{1A} is independently hydrogen.

In embodiments, R″ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{1B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{1B} is independently hydrogen.

In embodiments, R² is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, where R² is substituted, R² is substituted with a substituent group. In embodiments, where R² is substituted, R² is substituted with a size-limited substituent group. In embodiments, where R² is substituted, R² is substituted with a lower substituent group.

In embodiments, R^{2A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{2A} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{2A} is independently hydrogen.

In embodiments, R^{2B} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{2B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{2B} is independently hydrogen.

In embodiments, R³ is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, where R³ is substituted, R³ is substituted with a substituent group. In embodiments, where R³ is substituted, R³ is substituted with a size-limited substituent group. In embodiments, where R³ is substituted, R³ is substituted with a lower substituent group.

In embodiments, R^{3A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{3A} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{3A} is independently hydrogen.

In embodiments, R^{3B} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{3B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{3B} is independently hydrogen.

In embodiments, R⁴ is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, where R⁴ is substituted, R⁴ is substituted with a substituent group. In embodiments, where R⁴ is substituted, R⁴ is substituted with a size-limited substituent group. In embodiments, where R⁴ is substituted, R⁴ is substituted with a lower substituent group.

In embodiments, R^{4A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{4A} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{4A} is independently hydrogen.

In embodiments, R^{4B} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{4B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{4B} is independently hydrogen.

In embodiments, R⁵ is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, where R⁵ is substituted, R⁵ is substituted with a substituent group. In embodiments, where R⁵ is substituted, R⁵ is substituted with a size-limited substituent group. In embodiments, where R⁵ is substituted, R⁵ is substituted with a lower substituent group.

In embodiments, R^{5A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{5A} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{5A} is independently hydrogen.

In embodiments, R^{5B} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{5B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{5B} is independently hydrogen.

In embodiments, R⁶ is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, where R⁶ is substituted, R⁶ is substituted with a substituent group. In embodiments, where R⁶ is substituted, R⁶ is substituted with a size-limited substituent group. In embodiments, where R⁶ is substituted, R⁶ is substituted with a lower substituent group.

In embodiments, R^{6A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{6A} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{6A} is independently hydrogen.

In embodiments, R^{6B} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R^{6B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R^{6B} is independently hydrogen.

In embodiments, provided herein is a process for the preparation of compound of Formula II, or a salt thereof. In embodiments, the preparation of compound of Formula II, or a salt thereof, includes (a) contacting a compound of Formula III:

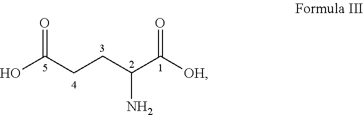

or a salt thereof, with a compound of Formula IV:

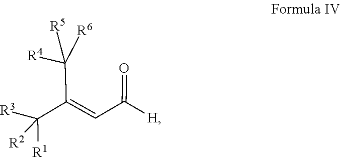

or a salt thereof, to form an imine; and (b) reducing the imine to produce the compound of Formula II. R¹, R², R³, R⁴, R⁵, and R⁶ are as described herein, including embodiments.

In embodiments, the process for the preparation of compound of Formula II (prekainic acid), or a salt thereof, includes the incubation of compound of Formula III (glutamic acid) with compound of Formula IV (3-methyl-2-butenal).

Particular process conditions, including one or more of reaction time, temperature, solvent, reagent, amount of reagent(s) order of reagent addition, pH etc. may be selected in order to optimize reaction product purity and/or yield.

In embodiments, compound of Formula III is in an aqueous solution. In embodiments, a base is added to aqueous solution of compound of Formula III. In embodiments, the base is lithium hydroxide, sodium hydroxide, potassium hydroxide, rubidium hydroxide, cesium hydroxide, calcium hydroxide, strontium hydroxide, or barium hydroxide. In embodiments the base is sodium hydroxide.

In embodiments, compound of Formula IV is added to the basic aqueous solution of compound of Formula III. In embodiments, the incubation is carried out for about 1 minute, 5 minutes, about 10 minutes, about 15, minutes, about 20 minutes, about 25 minutes, about 30 minutes, about 40 minutes, about 50 minutes, or about 60 minutes. In embodiments, the incubation is carried out at about −10° C., about −5° C., about 0° C., about 5° C., about 10° C., about 15° C., or about 20° C.

In embodiments, following the incubation of compound of Formula IV with basic aqueous solution of compound of Formula III the formed imine is reduced with a base. In embodiments, the imine reducing base is a metal borohydride. In embodiments, the metal borohydride is lithium borohydride, zinc borohydride or sodium borohydride. In embodiments, the imine reducing base is sodium borohydride, lithium borohydride, zinc borohydride, sodium cyanoborohydride, sodium tri-acetoxyborohydride or lithium aluminum hydride.

In another aspect, provided herein is a process for the preparation of a compound with the formula,

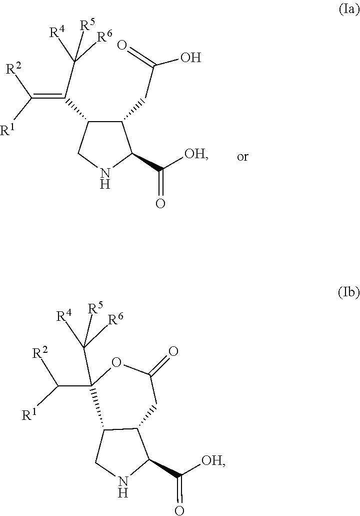

or a salt thereof. R¹, R², R³, R⁴, R⁵, and R⁶ are as described herein, including embodiments.

The compound or a salt of Formula (Ia) or (Ib) is prepared by contacting a compound of Formula III:

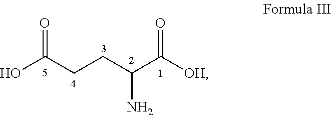

or a salt thereof, with a compound of Formula V:

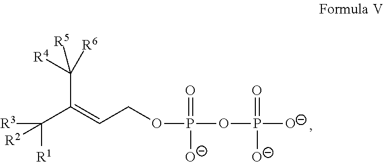

or a salt thereof, a recombinant KabA polypeptide, a recombinant KabC polypeptide, and a α-keto-glutarate. R¹, R², R³, R⁴, R⁵, and R⁶ are as described herein, including embodiments.

In embodiments, compound of Formula II, or a salt thereof, is prepared by contacting a compound of Formula III:

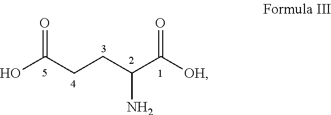

or a salt thereof, with a compound of Formula V:

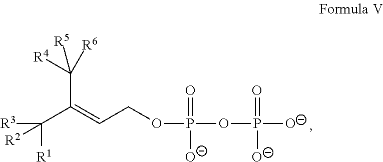

or a salt thereof, and a recombinant KabA polypeptide. R¹, R², R³, R⁴, R⁵, and R⁶ are as described herein, including embodiments.

In embodiments, the process for the preparation of compound of Formula (Ia) or (Ib), or a salt thereof, includes the incubation of compound of Formula III (glutamic acid) with compound of Formula V (DMAPP), D. simplex KabC (DsKabC), D. simplex KabA (DsKabA), α-keto-glutarate, L-ascorbate, Mg⁺² cofactor, and ferrous iron.

Particular process conditions, including one or more of reaction time, temperature, solvent, reagent, amount of reagent(s) order of reagent addition, pH etc. may be selected in order to optimize reaction product purity and/or yield.

In embodiments, ferrous iron is ferrous hydroxide, ferrous halide, ferrous nitrate, ferrous sulfate, ferrous oxide, ferrous carbonate, ferrous acetate, ferrous chromate. In embodiments, ferrous iron is ferrous sulfate.

In embodiments, Mg⁺² cofactor is magnesium halide, magnesium sulfate, magnesium nitrate, magnesium carbonate, or magnesium acetate. In embodiments, Mg⁺² cofactor is magnesium halide. In embodiments, Mg⁺² cofactor is magnesium chloride.

In embodiments, the incubation is carried out in a buffer at pH of about 5.0, about 5.5, about 6.0, about 6.5, about 7.0, about 7.5, about 8.0, about 8.5, about 9.0, about 9.5, or about 10.0. In embodiments, the incubation is carried out in a buffer of 300 mM potassium chloride and 50 mM HEPES pH 8.0. In embodiments, the incubation is carried out at about 5° C., about 10° C., about 15° C., about 20° C., about 25° C., about 30° C., about 35° C., about 40° C., or about 45° C. In embodiments, the incubation is carried out at about 22° C. In embodiments, the incubation is carried out for about 5 hours, about 10 hours, about 15 hours, about 20 hours, about 25 hours, about 30 hours, about 35 hours, about 40 hours, or about 45 hours. In embodiments, the incubation is carried out for about 18 hours.

In an aspect, provided herein is a compound of formula II:

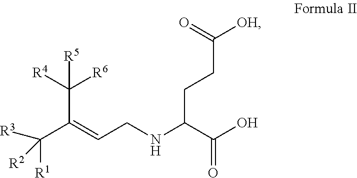

or a salt thereof. R¹, R², R³, R⁴, R⁵, and R⁶ are as described herein, including embodiments.

In an aspect, provided herein is a compound of formula VI:

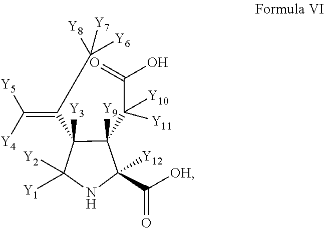

or a salt thereof. In Formula VI, one of Y₁, Y₂, Y₃, Y₄, Y₅, Y₆, Y₇, Y₈, Y₉, Y₁₀, Y₁₁, or Y₁₂ is a hydrogen that is isotopically enriched with deuterium or tritium. In embodiments, one of Y₁, Y₂, Y₃, Y₄, Y₅, Y₆, Y₇, Y₈, Y₉, Y₁₀, Y₁₁, or Y₁₂ is a hydrogen that is isotopically enriched with deuterium. one of Y₁, Y₂, Y₃, Y₄, Y₅, Y₆, Y₇, Y₈, Y₉, Y₁₀, Y₁₁, or Y₁₂ is a hydrogen that is isotopically enriched with tritium.

In embodiments, any two of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any three of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any four of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any five of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any six of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any seven of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any eight of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any nine of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any ten of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, any eleven of Y₁, Y₂, Y₃, Y₄, Y₅, Y₆, Y₇, Y₈, Y₉, Y₁₀, Y₁₁, or Y₁₂ are hydrogens that are isotopically enriched with deuterium or tritium. In embodiments, all of Y₁, Y₂, Y₃, Y₄, Y₅, Y₆, Y₇, Y₈, Y₉, Y₁₀, Y₁₁, and Y₁₂ are hydrogens that are isotopically enriched with deuterium or tritium.

In embodiments, any two of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any three of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any four of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any five of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any six of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any seven of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any eight of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any nine of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any ten of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, any eleven of Y₁, Y₂, Y₃, Y₄, Y₅, Y₆, Y₇, Y₈, Y₉, Y₁₀, Y₁₁, or Y₁₂ are hydrogens that are isotopically enriched with deuterium. In embodiments, all of Y₁, Y₂, Y₃, Y₄, Y₅, Y₆, Y₇, Y₈, Y₉, Y₁₀, Y₁₁, or Y₁₂ are hydrogens that are isotopically enriched with deuterium.

In embodiments, any two of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any three of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any four of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any five of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any six of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any seven of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any eight of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any nine of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any ten of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, any eleven of Y₁, Y₂, Y₃, Y₄, Y₅, Y₆, Y₇, Y₈, Y₉, Y₁₀, Y₁₁, or Y₁₂ are hydrogens that are isotopically enriched with tritium. In embodiments, all of Y₁, Y₂, Y₃, Y₄, Y₅, Y₆, Y₇, Y₈, Y₉, Y₁₀, Y₁₁, or Y₁₂ are hydrogens that are isotopically enriched with tritium.

In an aspect, provided herein is a compound of formula VII:

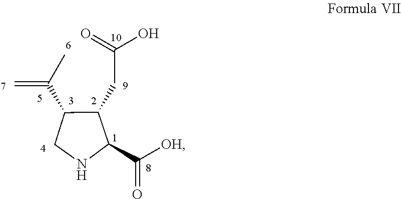

or a salt thereof. In Formula VII, 1, 2, 3, 4, 5, 6, 7, 8, 9, and 10 are carbon atoms, wherein at least one of 1, 2, 3, 4, 5, 6, 7, 8, 9, or 10 carbon atoms is isotopically enriched with carbon-13 or carbon-14. In embodiments, at least one of 1, 2, 3, 4, 5, 6, 7, 8, 9, or 10 carbon atoms is isotopically enriched with carbon-13. In embodiments, at least one of 1, 2, 3, 4, 5, 6, 7, 8, 9, or 10 carbon atoms is isotopically enriched with carbon-14.

In embodiments, any two of 1, 2, 3, 4, 5, 6, 7, 8, 9, or 10 carbon atoms are isotopically enriched with carbon-13 or carbon-14. In embodiments, any three of 1, 2, 3, 4, 5, 6, 7, 8, 9, or 10 carbon atoms are isotopically enriched with carbon-13 or carbon-14. In embodiments, any four of 1, 2, 3, 4, 5, 6, 7, 8, 9, or 10 carbon atoms are isotopically enriched with carbon-13 or carbon-14. In embodiments, any five of 1, 2, 3, 4, 5, 6, 7, 8, 9, or 10 carbon atoms are isotopically enriched with carbon-13 or carbon-14. In embodiments, any six of 1, 2, 3, 4, 5, 6, 7, 8, 9, or 10 carbon atoms are isotopically enriched with carbon-13 or carbon-14. In embodiments, any seven of 1, 2, 3, 4, 5, 6, 7, 8, 9, or 10 carbon atoms are isotopically enriched with carbon-13 or carbon-14. In embodiments, any eight of 1, 2, 3, 4, 5, 6, 7, 8, 9, or 10 carbon atoms are isotopically enriched with carbon-13 or carbon-14. In embodiments, any nine of 1, 2, 3, 4, 5, 6, 7, 8, 9, or 10 carbon atoms are isotopically enriched with carbon-13 or carbon-14. In embodiments, all carbon atoms are isotopically enriched with carbon-13 or carbon-14.

In embodiments, any two of 1, 2, 3, 4, 5, 6, 7, 8, 9, or 10 carbon atoms are isotopically enriched with carbon-13. In embodiments, any three of 1, 2, 3, 4, 5, 6, 7, 8, 9, or 10 carbon atoms are isotopically enriched with carbon-13. In embodiments, any four of 1, 2, 3, 4, 5, 6, 7, 8, 9, or 10 carbon atoms are isotopically enriched with carbon-13. In embodiments, any five of 1, 2, 3, 4, 5, 6, 7, 8, 9, or 10 carbon atoms are isotopically enriched with carbon-13. In embodiments, any six of 1, 2, 3, 4, 5, 6, 7, 8, 9, or 10 carbon atoms are isotopically enriched with carbon-13. In embodiments, any seven of 1, 2, 3, 4, 5, 6, 7, 8, 9, or 10 carbon atoms are isotopically enriched with carbon-13. In embodiments, any eight of 1, 2, 3, 4, 5, 6, 7, 8, 9, or 10 carbon atoms are isotopically enriched with carbon-13. In embodiments, any nine of 1, 2, 3, 4, 5, 6, 7, 8, 9, or 10 carbon atoms are isotopically enriched with carbon-13. In embodiments, all carbon atoms are isotopically enriched with carbon-13.

In embodiments, any two of 1, 2, 3, 4, 5, 6, 7, 8, 9, or 10 carbon atoms are isotopically enriched with carbon-14. In embodiments, any three of 1, 2, 3, 4, 5, 6, 7, 8, 9, or 10 carbon atoms are isotopically enriched with carbon-14. In embodiments, any four of 1, 2, 3, 4, 5, 6, 7, 8, 9, or 10 carbon atoms are isotopically enriched with carbon-14. In embodiments, any five of 1, 2, 3, 4, 5, 6, 7, 8, 9, or 10 carbon atoms are isotopically enriched with carbon-14. In embodiments, any six of 1, 2, 3, 4, 5, 6, 7, 8, 9, or 10 carbon atoms are isotopically enriched with carbon-14. In embodiments, any seven of 1, 2, 3, 4, 5, 6, 7, 8, 9, or 10 carbon atoms are isotopically enriched with carbon-14. In embodiments, any eight of 1, 2, 3, 4, 5, 6, 7, 8, 9, or 10 carbon atoms are isotopically enriched with carbon-14. In embodiments, any nine of 1, 2, 3, 4, 5, 6, 7, 8, 9, or 10 carbon atoms are isotopically enriched with carbon-14. In embodiments, all carbon atoms are isotopically enriched with carbon-14.

In an aspect, provided herein is a compound of formula VIII:

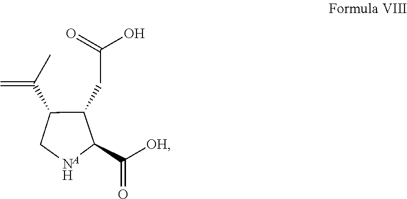

or a salt thereof. In Formula VIII, N^{A} is a nitrogen atom which is isotopically enriched with nitrogen-15.

In some embodiments, a process of preparing a kainic acid (e.g., a compound of Formula Ia or Ib) comprises contacting one or more compounds disclosed herein with a KabC and/or a KabA polypeptide, or a composition comprising a KabC polypeptide and/or a KabA polypeptide, optionally in the presence of one or more substrates, additives or salts.

In some embodiments, a process of preparing a kainic acid (e.g., a compound of Formula Ia or Ib) comprises contacting one or more compounds disclosed herein with a KabC polypeptide or a composition comprising a KabC polypeptide, and optionally one or more substrates (e.g., α-keto-glutarate (aKG)). In some embodiments, a process comprises cyclizing a compound disclosed herein, wherein the process comprises contacting a compound disclosed herein with a KabC polypeptide or a composition comprising a KabC polypeptide. In some embodiments, a compound of Formula II, or a salt thereof, is contacted with a KabC polypeptide, or a composition comprising a KabC polypeptide. In some embodiments, a compound of Formula III, or a salt thereof, is contacted with a KabC polypeptide, or a composition comprising a KabC polypeptide. In some embodiments, a compound of Formula V, or a salt thereof, is contacted with a KabC polypeptide, or a composition comprising a KabC polypeptide. In some embodiments, a compound of Formula III, or a salt thereof, and a compound of Formula V, or a salt thereof, is contacted with a KabC polypeptide, or a composition comprising a KabC polypeptide. In certain embodiments, a compound disclosed herein is contacted with a KabC polypeptide, or a composition comprising a KabC polypeptide, thereby forming a compound of Formula Ia or Ib, or a salt thereof.

In some embodiments, a process of preparing a kainic acid (e.g., a compound of Formula Ia or Ib) comprises contacting a compound disclosed herein with a KabA polypeptide or a composition comprising a KabA polypeptide, and optionally one or more substrates (e.g., α-keto-glutarate (αKG)). In some embodiments, a process comprises cyclizing a compound disclosed herein, wherein the process comprises contacting a compound disclosed herein with a KabA polypeptide or a composition comprising a KabA polypeptide. In certain embodiments, a compound disclosed herein is contacted with a KabA polypeptide, or a composition comprising a KabA polypeptide, thereby forming a compound of Formula Ia or Ib, or a salt thereof. In some embodiments, a compound of Formula II, or a salt thereof, is contacted with a KabA polypeptide, or a composition comprising a KabA polypeptide. In some embodiments, a compound of Formula III, or a salt thereof, is contacted with a KabA polypeptide, or a composition comprising a KabA polypeptide. In some embodiments, a compound of Formula V, or a salt thereof, is contacted with a KabA polypeptide, or a composition comprising a KabA polypeptide. In some embodiments, a compound of Formula III, or a salt thereof, and a compound of Formula V, or a salt thereof, is contacted with a KabA polypeptide, or a composition comprising a KabA polypeptide.

In some embodiments, a process of preparing a kainic acid (e.g., a compound of Formula Ia or Ib) comprises contacting a compound disclosed herein with a KabC polypeptide and/or a KabA polypeptide. In some embodiments, one or more compounds disclosed herein are contacted with a KabC polypeptide and/or a KabA polypeptide and a substrate, non-limiting examples of which include one or more of α-keto-glutarate (αKG), L-glutamate, glucose, dimethylallyl pyrophosphate (DMAPP), dimethylallyl diphosphate (DMADP), L-ascorbate, magnesium and iron, the like, a salt thereof or a combination thereof. In some embodiments, one or more compounds disclosed herein are contacted with a KabC polypeptide and/or a KabA polypeptide, L-glutamate and DMAPP. In some embodiments, one or more compounds disclosed herein are contacted with a KabC polypeptide and/or a KabA polypeptide and α-keto-glutarate. For example, in certain embodiments, a compound of Formula II, or a salt thereof, is contacted with a KabC polypeptide or a composition comprising a KabC polypeptide and α-keto-glutarate. In certain embodiments, a process comprises contacting a compound of Formula III, or a salt thereof, and a compound of Formula V, or a salt thereof, with a KabC polypeptide, a KabA polypeptide and α-keto-glutarate.

In some embodiments, a KabC polypeptide and/or a KabA polypeptide, or a composition comprising a KabC polypeptide and/or a KabA polypeptide, is contacted with DMAPP and L-glutamate. In some embodiments, a KabC polypeptide and/or a KabA polypeptide, or a composition comprising a KabC polypeptide and/or a KabA polypeptide, is contacted with DMAPP, L-glutamate and αKG, optionally in the presence of one or more of iron, magnesium and/or oxygen.

In some embodiments a composition comprising a KabC polypeptide and/or a KabA polypeptide comprises a crude, partially purified or purified KabC or KabA polypeptide. In some embodiments a composition comprising KabC polypeptide and/or a KabA polypeptide comprises a crude extract (e.g., a cell lysate) or conditioned media comprising a KabC polypeptide and/or a KabA polypeptide. In some embodiments, a composition comprising a KabC polypeptide and/or a KabA polypeptide comprises one or more recombinant organisms, for example a genetically modified microorganism engineered to express a recombinant KabC and/or KabA polypeptide. Accordingly, in certain embodiments, a process comprises contacting one or more compounds and/or substrates disclosed herein with one or more microorganisms (e.g., bacteria or yeast) that express a KabC and/or KabA polypeptide. In some embodiments, a process comprises contacting one or more compounds and/or substrates disclosed herein with a recombinant E. coli that expresses a recombinant KabC and/or a KabA polypeptide.

In some embodiments, a process of making a compound disclosed herein comprises culturing, propagating or growing a recombinant cell described herein in a suitable culture medium. In some embodiments, a process of making a compound disclosed herein (e.g., a compound of Formula Ia or Ib) comprises culturing, growing or expanding one or more recombinant cells configured to express a KabC and/or a KabA polypeptide, and contacting the cultured recombinant cells with a compound of Formula II, III, IV, V, a salt thereof, or a combination thereof. In some embodiments, a process of making a compound disclosed herein comprises culturing one or more recombinant cells configured to express a KabC and/or a KabA polypeptide, and contacting the cultured recombinant cells with a compound of Formula II, III, IV, V, a salt thereof, or a combination thereof, and one or more substrates (e.g., selected from α-keto-glutarate (αKG), L-glutamate, glucose, dimethylallyl pyrophosphate (DMAPP), dimethylallyl diphosphate (DMADP), L-ascorbate, magnesium, iron, the like, and salts thereof). In certain embodiments, a process further comprises isolating or purifying a compound of Formula Ia or Ib, or a salt thereof, from a culture medium (i.e., conditioned media) or from a recombinant cell described herein using a suitable method.

In some embodiments, a partially purified KabC and/or KabA polypeptide is in a range of 10%-90% pure, 10%-80% pure, 10%-60% pure or 30% to 60% pure. In certain embodiments, a purified KabC and/or KabA polypeptide is at least 75% pure, at least 80% pure, at least 90% pure, at least 95% pure or at least 98% pure. In some embodiments, a purified KabC and/or a purified KabA polypeptide is 90-100%, 95-100% pure or 98-100% pure.

In some embodiments, a KabC and/or KabA polypeptide is a recombinant polypeptide. A recombinant KabC and/or KabA polypeptide can be expressed by a suitable recombinant organism, non-limiting examples of which include a genetically modified plant, algae, mammalian cell, bacteria or yeast. In some embodiments, a recombinant KabC and/or KabA polypeptide can be expressed by a suitable recombinant microorganism, non-limiting examples of which include a genetically modified bacteria or yeast. An organism can be genetically modified using any suitable method, system or vector. For example, a microorganism can be genetically modified by stable or transient integration of a recombinant polynucleotide (e.g., a recombinant expression vector) using a suitable vector or delivery system, non-limiting examples of which include a virus, phage, plasmid, cosmid, BAC, CRISPR element, the like or combinations thereof.

In some embodiments, a recombinant KabC and/or KabA polypeptide is expressed by a suitable expression system non-limiting examples of which include an insect expression system (e.g., baculovirus mediated expression), a plant expression system, an algae expression system, a mammalian expression system, a yeast expression system and a prokaryotic (e.g., bacterial) expression system. In some embodiments, a recombinant KabC and/or KabA polypeptide is generated, expressed and/or produced by a suitable bacteria. In certain embodiments, a recombinant KabC and/or KabA polypeptide is generated, expressed and/or produced by a recombinant bacteria of the genus Escherichia. In certain embodiments, a recombinant KabC and/or KabA polypeptide is generated, expressed and/or produced by a recombinant Escherichia coli (E. coli), or a suitable strain thereof.

In some embodiments, presented herein is recombinant polynucleotide comprising a polynucleotide encoding a KabA polypeptide and/or a KabC polypeptide. In some embodiments, presented herein is an expression vector comprising a recombinant polynucleotide encoding a KabA polypeptide and/or a KabC polypeptide. In some embodiments, an expression vector comprises a polynucleotide encoding a KabA polypeptide and a KabC polypeptide. In some embodiments, an expression vector comprises one or more polynucleotides selected from SEQ ID NOs:6-10 and 33-34. In certain embodiments, an expression vector disclosed herein is configured to express a KabA polypeptide and/or a KabC polypeptide in a suitable organism, microorganism or expression system. Accordingly, an expression vector often comprises one or more suitable promoters that are operably linked to a polynucleotide encoding a KabA polypeptide and/or KabC polypeptide.

In some embodiments, presented herein is a suitable recombinant cell comprising a polynucleotide encoding a KabA polypeptide and/or a KabC polypeptide. In some embodiments, a recombinant cell is configured to express a KabA polypeptide and/or a KabC polypeptide. In some embodiments, a recombinant cell comprises an expression vector described herein. In certain embodiments, a recombinant cell is a genetically modified mammalian, insect, plant, yeast or prokaryotic cell that is configured to express a KabA polypeptide and/or a KabC polypeptide. In certain embodiments, a recombinant cell is a bacterial cell. In some embodiments a recombinant cell is a recombinant or genetically modified E. coli.

In some embodiments, a KabC polypeptide comprises an amino acid sequence that is 70%-100%, 80%-100%, 90%-100%, 95%-100% or 98%-100% identical to the amino acid sequence of any one of SEQ ID NOs:1-5, a conservatively modified variant thereof, or an enzymatically active portion thereof. A KabC polypeptide that is 70%-100%, 80%-100%, 90%-100%, 95%-100% or 98%-100% identical to the amino acid sequence of any one of SEQ ID NOs:1-5, or a portion thereof, may comprise non-identical amino acids that are conservative substitutions. Accordingly, a KabC polypeptide described herein is sometimes a conservatively modified variant of KabC polypeptide of any one of SEQ ID NOs:1-5. In certain embodiments, a KabC polypeptide comprises an amino acid sequence that is at least 70%, at least 75%, at least 80%, at least 85%, at least 90%, at least 90% or at least 95% identical to the amino acid sequence of any one of SEQ ID NOs:1-5. In embodiments, the KabC polypeptide comprises an amino acid sequence that is at least 80% identical to the amino acid sequence of any one of SEQ ID NOs: 1-5. In embodiments, the KabC polypeptide comprises an amino acid sequence that is at least 90% identical to the amino acid sequence of any one of SEQ ID NOs: 1-5. In embodiments, the KabC polypeptide comprises an amino acid sequence that is at least 95% identical to the amino acid sequence of any one of SEQ ID NOs: 1-5. In certain embodiments, a KabC polypeptide comprises an amino acid sequence of any one of SEQ ID NOs: 1-5. In embodiments, the KabC polypeptide comprises an amino acid sequence of SEQ ID NO: 1, or a conservatively modified variant thereof. In embodiments, the KabC polypeptide comprises an amino acid sequence of SEQ ID NO: 2, or a conservatively modified variant thereof. In embodiments, the KabC polypeptide comprises an amino acid sequence of SEQ ID NO: 3, or a conservatively modified variant thereof. In embodiments, the KabC polypeptide comprises an amino acid sequence of SEQ ID NO: 4, or a conservatively modified variant thereof. In embodiments, the KabC polypeptide comprises an amino acid sequence of SEQ ID NO: 5, or a conservatively modified variant thereof.

In some embodiments, a KabC polypeptide comprises an amino acid sequence encoded by a polynucleotide comprising a sequence that is 70%-100%, 80%-100%, 90%-100%, 95%-100% or 98%-100% identical to the polynucleotide sequence of any one of SEQ ID NOs:6-10 and 33-34, or a portion thereof. In certain embodiments, a KabC polypeptide comprises an amino acid sequence encoded by a polynucleotide that is at least 70%, at least 75%, at least 80%, at least 85%, at least 90%, at least 90% or at least 95% identical to the polynucleotide sequence of any one of SEQ ID NOs:6-10 and 33-34. In embodiments, the polynucleotide comprises a sequence that is at least about 80% identical to any one of SEQ ID NOs: 6-10 and 33-34. In embodiments, the polynucleotide comprises a sequence that is at least about 90% identical to any one of SEQ ID NOs: 6-10 and 33-34. In embodiments, the polynucleotide comprises a sequence that is at least about 95% identical to any one of SEQ ID NOs: 6-10 and 33-34. In certain embodiments, a KabC polypeptide is encoded by a polynucleotide sequence of any one of SEQ ID NOs:6-10 and 33-34.

In some embodiments, a KabA polypeptide comprises an amino acid sequence that is 70%-100%, 80%-100%, 90%-100%, 95%-100% or 98%-100% identical to the amino acid sequence of any one of SEQ ID NOs:11-15, a conservatively modified variant thereof, or an enzymatically active portion thereof. A KabA polypeptide that is 70%-100%, 80%-100%, 90%-100%, 95%-100% or 98%-100% identical to the amino acid sequence of any one of SEQ ID NOs: 11-15, or a portion thereof, may comprise non-identical amino acids that are conservative substitutions. Accordingly, a KabA polypeptide described herein is sometimes a conservatively modified variant of KabA polypeptide of any one of SEQ ID NOs:11-15. In certain embodiments, a KabA polypeptide comprises an amino acid sequence that is at least 70%, at least 75%, at least 80%, at least 85%, at least 90%, at least 90% or at least 95% identical to the amino acid sequence of any one of SEQ ID NOs: 11-15. In embodiments, the KabA polypeptide comprises an amino acid sequence that is at least 80% identical to the amino acid sequence of any one of SEQ ID NOs: 11-15. In embodiments, the KabA polypeptide comprises an amino acid sequence that is at least 90% identical to the amino acid sequence of any one of SEQ ID NOs: 11-15. In embodiments, the KabA polypeptide comprises an amino acid sequence that is at least 95% identical to the amino acid sequence of any one of SEQ ID NOs: 11-15. In certain embodiments, a KabA polypeptide comprises an amino acid sequence of any one of SEQ ID NOs:11-20. In embodiments, the KabA polypeptide comprises an amino acid sequence of SEQ ID NO: 11, or a conservatively modified variant thereof. In embodiments, the KabA polypeptide comprises an amino acid sequence of SEQ ID NO: 12, or a conservatively modified variant thereof. In embodiments, the KabA polypeptide comprises an amino acid sequence of SEQ ID NO: 13, or a conservatively modified variant thereof. In embodiments, the KabA polypeptide comprises an amino acid sequence of SEQ ID NO: 14, or a conservatively modified variant thereof. In embodiments, the KabA polypeptide comprises an amino acid sequence of SEQ ID NO: 15, or a conservatively modified variant thereof.

In some embodiments, a KabA polypeptide comprises an amino acid sequence encoded by a polynucleotide comprising a sequence that is 70%-100%, 80%-100%, 90%-100%, 95%-100% or 98%-100% identical to the polynucleotide sequence of any one of SEQ ID NOs:16-20, or a portion thereof. In certain embodiments, a KabA polypeptide comprises an amino acid sequence encoded by a polynucleotide that is at least 70%, at least 75%, at least 80%, at least 85%, at least 90%, at least 90% or at least 95% identical to the polynucleotide sequence of any one of SEQ ID NOs:16-20. In embodiments, the polynucleotide comprises a sequence that is at least about 80% identical to any one of SEQ ID NOs:16-20. In embodiments, the polynucleotide comprises a sequence that is at least about 90% identical to any one of SEQ ID NOs:16-20. In embodiments, the polynucleotide comprises a sequence that is at least about 95% identical to any one of SEQ ID NOs:16-20. In certain embodiments, a KabA polypeptide is encoded by a polynucleotide sequence of any one of SEQ ID NOs:16-20.

In certain embodiments, a KabC or KabA polypeptide (or polynucleotide encoding the same) is obtained or derived from algae (e.g., a red algae), comprising the sequence of the corresponding protein, or a conservatively modified variant thereof. In certain embodiments, a KabC or KabA polypeptide (or a polynucleotide encoding the same) is obtained or derived from an organism of the class Florideophyceae. In certain embodiments, a KabC or KabA polypeptide (or polynucleotide encoding the same) is obtained or derived from an organism of the genus Digenea, Palmaria, Rhodophysema, or Grateloupia. In some embodiments, a KabC or KabA polypeptide (or polynucleotide encoding the same) is a Digenea spp. polypeptide, a Rhodophysema spp. polypeptide, a Palmaria spp. polypeptide or a Grateloupia spp. polypeptide. In certain embodiments, a KabC or KabA polypeptide (or polynucleotide encoding the same) is obtained or derived from Digenea simplex, Rhodophysema elegans, Palmaria palmata, Palmaria hecatensis or Grateloupia filicina. Additional algal sources of KabA and/or KabC are disclosed herein, including within the figures. In embodiments, the KabA and KabC are obtained or derived from the same genus or species. In embodiments, the KabA and KabC are obtained or derived from different genera or species.

### EXAMPLES

The following illustrative examples are representative of embodiments of compositions and methods described herein and are not meant to be limiting in any way.

### Example 1

### Genomic Sequencing and Gene Cluster Identification

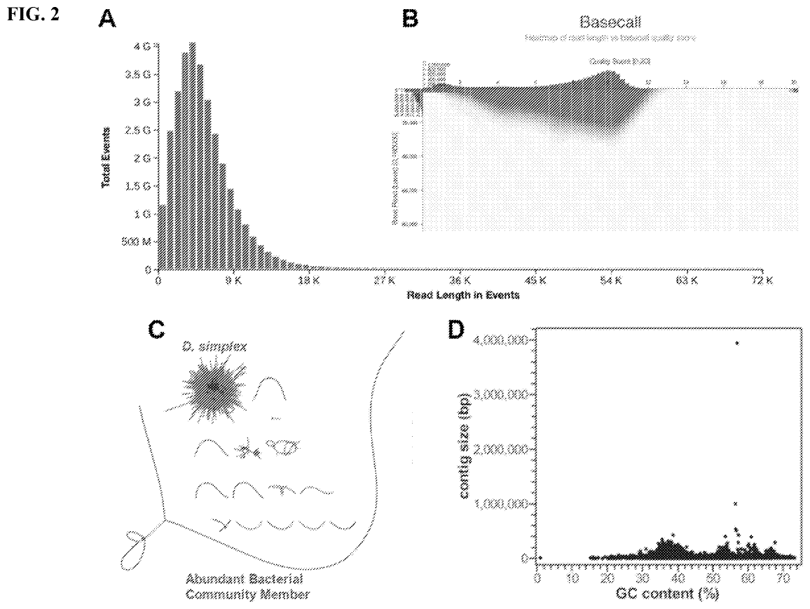

Whole genome sequencing was performed of the well-studied kainic acid producer D. simplex to identify the kainic acid biosynthetic genes and determine their genetic context. Red macroalgae genomes have proven challenging due to their close association with a diverse array of marine microbes and crustaceans, which means sequencing efforts result in highly complex metagenomes. Illumina short read sequencing results in assemblies of millions of small contigs making it difficult to impossible to resolve large genic blocks in red macroalgae genomes. Therefore, we sequenced D. simplex with the Oxford Nanopore Technology (ONT), which produces long, single molecule sequences that greatly facilitate assembly of large genomes [23]. While the D. simplex genome was predicted to be ˜200 Mb, the high microbial level in the isolated DNA was sequenced on the high throughput PromtheION ONT platform, producing 47 Gb of sequence. The resulting genome assembly was 299 Mb in length, with 8,246 contigs and the longest contig 3.9 Mb, with a contig N50 of 57 kb (FIG. 2). Based on GC % analysis, we predicted that 205 Mb of the assembly were found to be associated with D. simplex, while 88 Mb were associated with high QC % bacterial contigs.

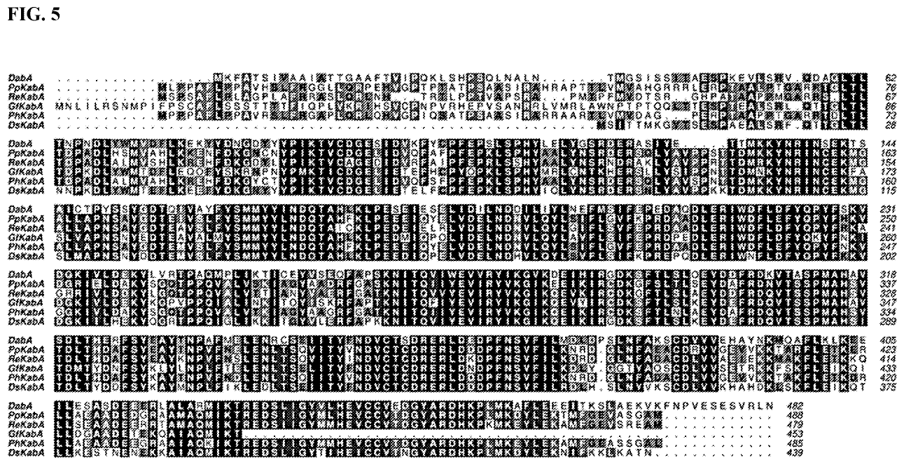

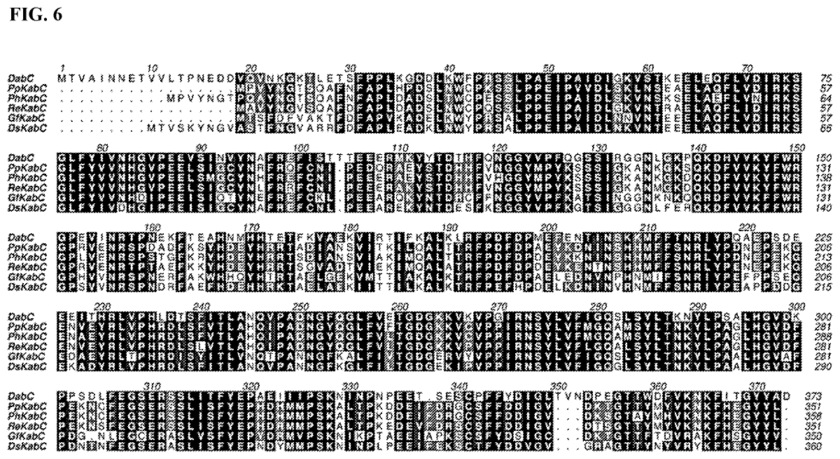

Upon analyzing the D. simplex genome for the presence of homologs to the domoic acid biosynthetic (dab) genes, we uncovered a 12 kb three-gene kainic acid biosynthesis (kab) cluster containing an annotated N-prenyltransferase, α-ketoglutarate (αKG) dependent dioxygenase, and several retrotransposable elements (integrase, reverse transcriptase, and RNase H domains) (FIG. 1, view (C)). Many organisms, including bacteria, fungi, and even plants, are known to cluster genes within the same biosynthetic pathway [24,25]. The kab gene clustering is consistent with early examples of red algae clustering genes [26] and suggested that it could also be a feature in red algal specialized metabolism. Analysis of the NCBI transcriptome database identified similar kab transcripts from four different red algae, namely Palmaria palmata, Grateloupia filicina, Palmaria hecatensis, and Rhodophysema elegans [27,28]. Notably, kainic acid has been previously isolated from P. palmata [29], while the other three red algae are not known producers. Sequence alignment of the KabA N-prenyltransferase and KabC αKG-dependent dioxygenase proteins revealed high levels of conservation (FIG. 5 and FIG. 6). Both the domoic acid N-prenyltransferase dabA [22] and most of the KabA proteins contain a non-conserved N-terminal region that may function as a signal peptide to localize the protein to a cellular compartment (FIG. 5). However, the D. simplex KabA (DskabA) lacks this sequence and is thus expected to remain within the cytosol.

Bundled between the D. simplex kabAC genes is a viral reverse transcriptase domain-containing gene, kabB. This association suggests that both kabA and kabC could be part of a mobile genetic element that could facilitate horizontal transfer between different algae and could potentially explain the mixed phylogenetic distribution of kainic acid biosynthetic gene containing red algae. Mobile genetic elements are common across plants, bacteria, and fungi and have been shown to be integral in meditating horizontal gene transfer of entire pathways [30]. To evaluate whether this feature is conserved within the kab clusters, we performed genome sequencing of a second known kainic acid producer, P. palmata, to determine the genetic context of its kab genes. As with D. simplex, the kainic acid genes were found to be tightly clustered. However, the gene synteny was not found to be conserved with only a short 1.2 kb intergenic region between ppkabA and ppkabC and with different gene orientation. Immediately outside the P. palmata cluster is a transposable element, which is distinct from the type observed in D. simplex. While this difference suggested that the retrotransposon was either not the mode of gene transport or that it had since been subsequently lost, more clusters could be studied to validate this trend.

TABLE S3
NCBI accession codes for deposited sequencing
data and previously published RNASeq data.

| | Organism | NCBI Accession Number |
|---|---|---|
| Digenea simplex bioproject | | PRJNA509898 |
| Digenea simplex hologenome | | RXNZ00000000 |
| Digenea simplex SRA | | SRR8325638, SRR8325637, |
| | | SRR8325636 |
| Digenea simplex kainic acid | | MK312630 |
| cluster | | |
| Palmaria palmata bioproject | | PRJNA509900 |
| Palmaria palmata SRA | | SRR8325558, SRR8325559 |
| Palmaria palmata kainic acid | | MK312631 |
| cluster | | |
| PpKabA from published | Palmaria palmata | QCC62375 |
| transcriptomics [28] | | |
| PpKabC from published | Palmaria palmata | QCC62376 |
| transcriptomics [28] | | |
| DsKabA | Digenea simplex | QCC62379 |
| DsKabC | Digenea simplex | QCC62383 |
| PpKabA | Palmaria palmata | QCC62384 |
| PpKabC | Palmaria palmata | QCC62385 |
| ReKabA | Rhodophysema elegans | QCC62371 |
| ReKabC | Rhodophysema elegans | QCC62372 |
| PhKabA | Palmaria hecatensis | QCC62373 |
| PhKabC | Palmaria hecatensis | QCC62374 |
| GfKabA (partial sequence) [27] | Grateloupia filicina | QCC62377 |
| GfKabC [27] | Grateloupia filicina | QCC62378 |

### Validation of the Kainic Acid Biosynthesis Enzymes KabA and KabC

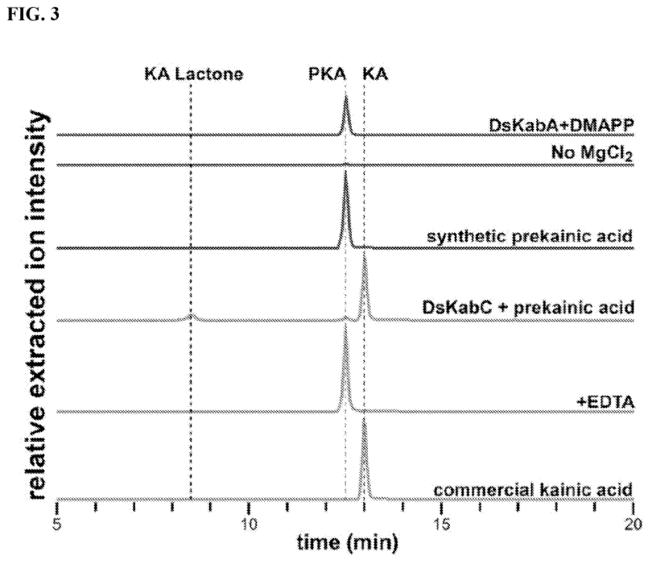

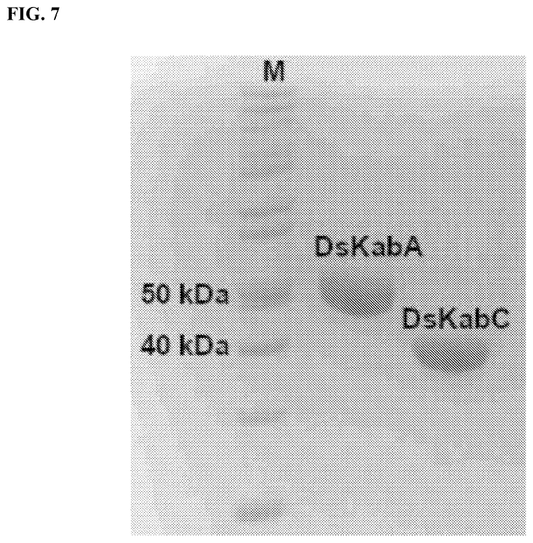

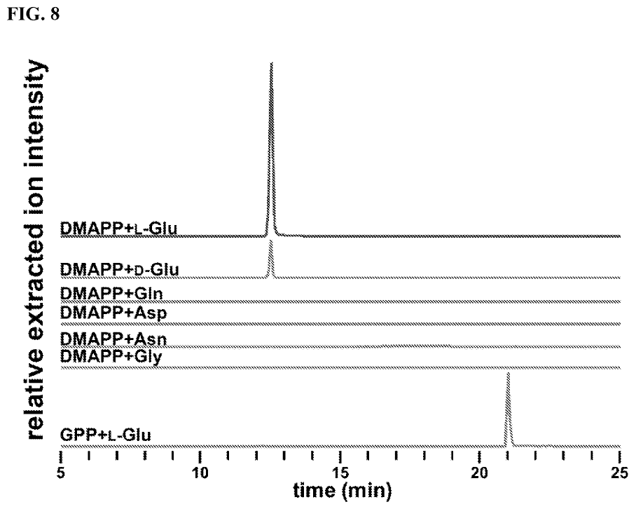

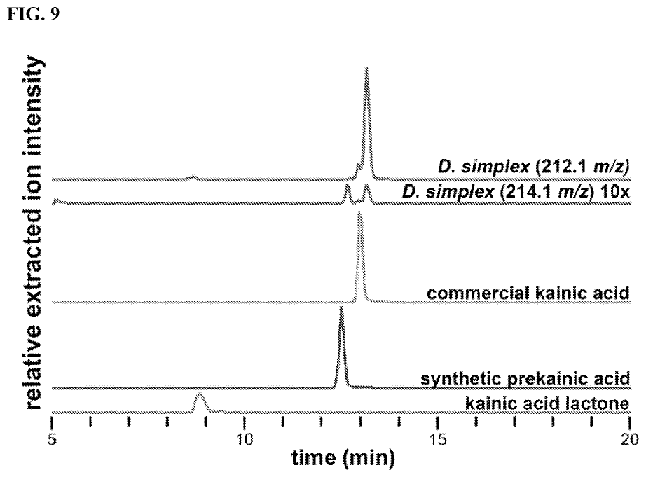

To confirm the proposed kainic acid biosynthetic pathway, dsKabA and dsKabC were successfully expressed in Escherichia coli and purified to homogeneity (FIG. 7). Incubation of DsKabA with the anticipated substrates DMAPP and L-glutamate produced N-dimethylallyl-L-glutamic acid (prekainic acid) as confirmed by NMR structural elucidation and LCMS comparison with a synthetic standard (FIG. 3). Consistent with other members of the terpene cyclase family fold, Mg²⁺ is a required cofactor in the reaction (FIG. 3). We next explored the substrate selectivity of DsKabA. Exchange of L-glutamate for D-glutamate showed diminished turnover while L-glutamine, L-aspartate, L-asparagine, and glycine did not produce appreciable amounts of N-prenylated product (FIG. 8). In contrast, when we replaced DMAPP with GPP in the reaction with L-glutamate, we observed the formation of N-geranyl-L-glutamic acid (L-NGG), the first biosynthetic precursor to domoic acid (FIG. 8). This high level of amino acid selectivity and modest prenyl donor promiscuity was also observed with DabA from domoic acid biosynthesis [22]. To give evidence to the physiological relevance of prekainic acid in the biosynthesis of kainic acid, we examined the aqueous extract of D. simplex. High resolution LCMS analysis revealed the presence of a compound with both the exact mass and retention time of prekainic acid (FIG. 9).

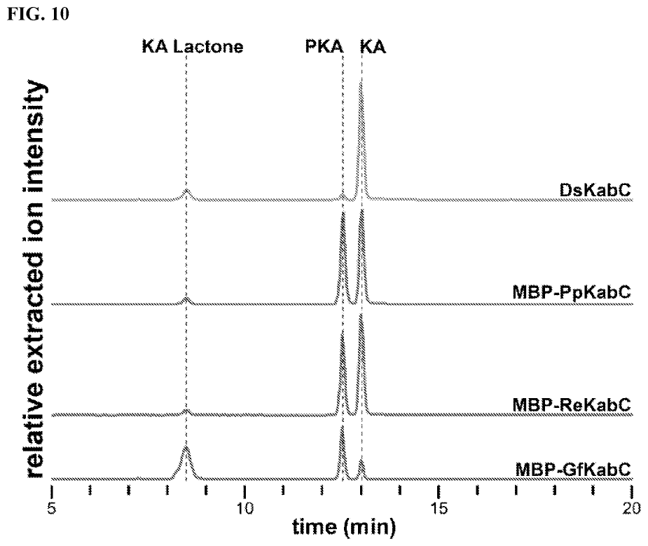

After reconstituting DsKabA activity, we next aimed to determine if DsKabC could directly convert prekainic acid to kainic acid. Incubation of DsKabC with prekainic acid, αKG, L-ascorbate, and Fe²⁺ resulted in the conversion of prekainic acid to two products with the expected mass of kainic acid (FIG. 3). Addition of the iron chelator EDTA nearly abolished activity. The major product had the same HPLC retention time and MS of commercially available kainic acid, and was further confirmed by to be kainic acid following NMR structural elucidation. Examination of the in vitro products of other algal KabC enzymes revealed a similar trend, wherein prekainic acid was converted to kainic acid and another minor isomeric product (FIG. 10). Notably, the KabC ortholog from G. filicinia, GfKabC, produced primarily this other kainic acid isomer. By scaling up the GfKabC reaction, we isolated and characterized this product by NMR to reveal kainic acid lactone (FIG. 1, view (B)). Kainic acid lactone has been previously isolated from kainic acid producing red algae [31] and shown to be an iGluR antagonist [32], in contrast to the agonistic activity of kainic acid. While kainic acid lactone has been isolated in small quantities from several red algae, it can be produced from kainic acid under acidic conditions, which prompted the initial concern that it may simply be an isolation artifact. However, the combination of our in vitro experiments and D. simplex distilled water extractions suggest that this bicyclic kainoid is an authentic natural product (FIG. 9). The relative ratio of kainic acid to kainic acid lactone appeared to vary depending on the particular KabC ortholog (FIG. 10).

### Scalable Bioproduction of Kainic Acid

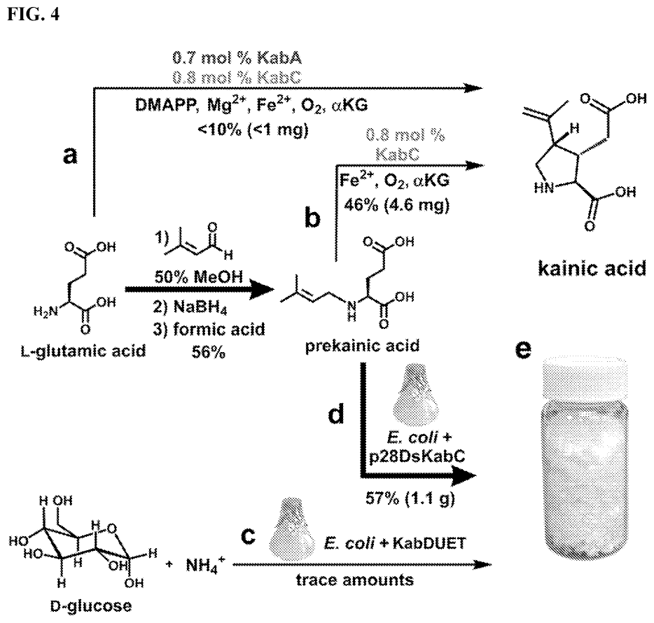

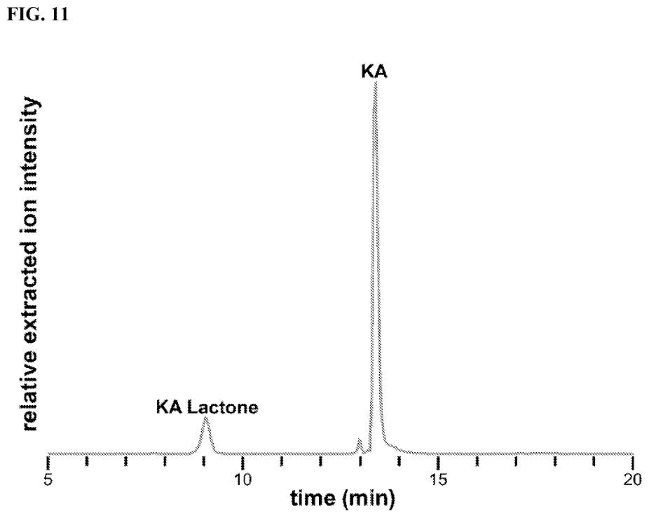

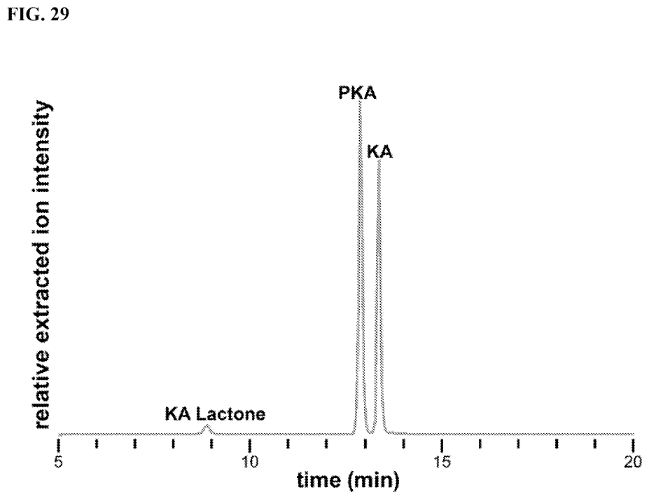

Once the Kab enzymatic functions were validated, we focused on the development of a practical kainic acid production system. As the biosynthesis requires only two enzymes, we initially envisioned two complementary in vitro production routes. First, we evaluated an enzymatic total biosynthesis wherein kainic acid is produced from the three substrates, DMAPP, L-Glu, and αKG (FIG. 4, view (a)). In a single pot reaction, we showed that recombinant DsKabA and DsKabC, each isolated from 1 L E. coli culture, could be coupled together to produce kainic acid. While kainic acid was successfully produced, the reaction did not go to completion and only afforded <1 mg of kainic acid with an approximate <10% yield (FIG. 29). As this procedure is also limited by the need to purify two enzymes, we developed a two-step chemoenzymatic method necessitating just one biocatalyst. We replaced KabA and its expensive DMAPP substrate with a simplified synthetic preparation of prekainic acid by reductive amination of L-glutamate with 3-methyl-2-butenal (FIG. 4, view (b)). Upon purification, synthetic prekainic acid was then completely enzymatically converted with DsKabC, producing 4.6 mg kainic acid with a 46% yield from 1 L of purified protein and a 26% overall yield starting from L-Glu (FIG. 11). Although the chemoenzymatic route was simpler, neither approach appeared to be readily applicable for large scale production of kainic acid.

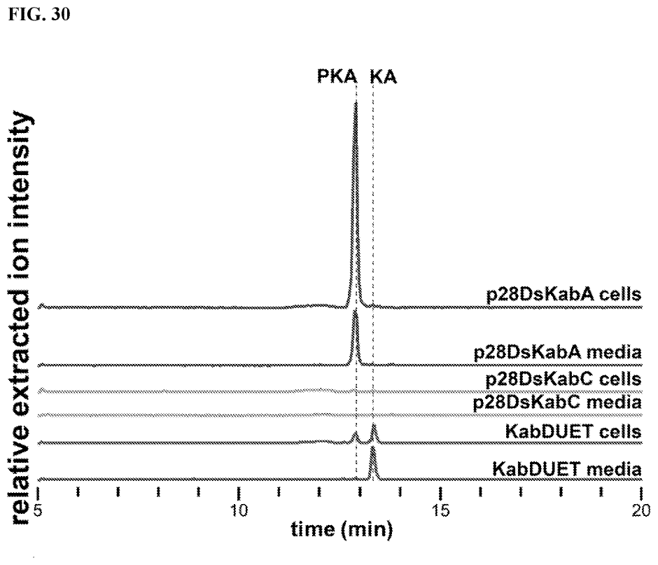

To complement the in vitro production methods, we next aimed to develop an E. coli fermentation system (FIG. 4, view (c)). As kainic acid is biosynthesized from the primary metabolites L-glutamate and DMAPP, we reasoned that co-expression of dskabA and dskabC as part of a pETDuet vector (KabDUET) should enable production of kainic acid. Examination of both the cell extract and fermentation media by LCMS, however, indicated only trace amounts of kainic acid (FIG. 30).

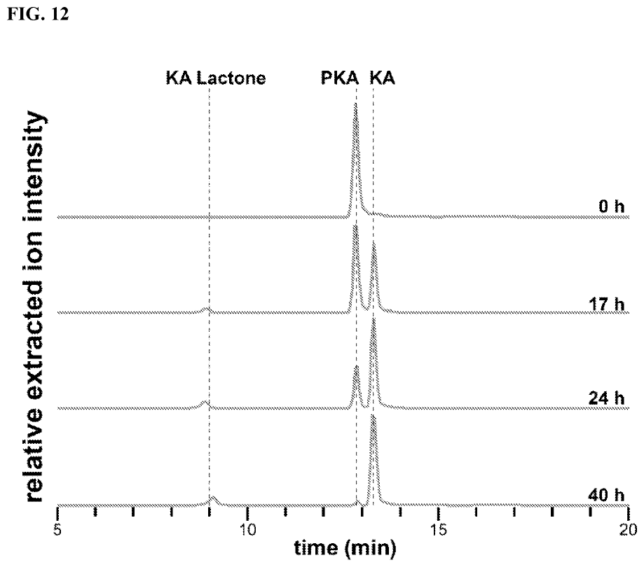

As the Kab enzymes appeared active within the E. coli fermentation conditions, we next evaluated a biotransformation strategy. We had earlier observed kainic acid present both within the cells and the media, while prekainic acid was not observed (FIG. 30). This observation suggested that DsKabA catalyzes the limiting reaction and that kainic acid can exchange between the media and cells by either active or passive transport. Therefore, we explored a biotransformation strategy with synthetic prekainic acid and E. coli cells expressing DsKabC (FIG. 4, view (d)). Initially we purified synthetic prekainic acid prior to addition to the E. coli culture, but ultimately, we developed a simpler strategy in which we added the entire unpurified crude reaction materials to the E. coli medium. After 40 h, we observed nearly complete consumption of an 8 mmol prekainic acid synthetic reaction in a 3×1 L E. coli biotransformation to primarily kainic acid and some kainic acid lactone (˜10% by ion intensity) (FIG. 12). Employing a simple two-step purification using activated carbon followed by preparatory reversed phase HPLC, we purified 1.1 g of kainic acid from 6 L of minimal media with a combined overall yield of 32% and >95% purity as assessed by NMR.

### DISCUSSION

As new DNA sequencing technologies are released, genomes of complex organisms can be assembled more quickly and at relatively low cost. This enables the opportunity to study underexplored classes of organisms. By focusing on a producer of the iconic natural product kainic acid, we have shown that biosynthetic genes can be clustered in red macroalgae. While the enzymatic activities were consistent with our initial hypothesis, the transformations they catalyze remain extremely unusual. N-prenylation is much less common than either C- or O-prenylation and typically utilizes a secondary amine instead of primary amine [35]. To our knowledge, KabA and DabA represent the first examples of N-prenylation of a free amino acid with intriguingly high specificity. Additionally, αKG dioxygenases are most known for hydroxylation, desaturation, chlorination, or epimerization chemistry [36]. The observed C—C bond formation by KabC and DabC was reportedly rare within this family of enzymes, with only a few examples [37,38].

In addition to simply discovering and validating the kainic acid biosynthetic cluster, the short route of biosynthesis presented an excellent opportunity to demonstrate the utility of a natural product biosynthetic enzyme to produce a fine chemical. The global emphasis on developing biocompatible green chemistry positions enzymes to be viable replacements for certain transformations [39]. In particular, reactions that require high levels of stereo- and regiospecificity are ideal candidates for enzymatic biocatalysts. This is exemplified in KabC, which catalyzes the formation of a five-membered ring while installing two stereocenters. Although there are already many scalable synthetic processes for kainic acid, the procedures generally require at least 6 synthetic steps with yields <40% [20,40] and produce environmentally toxic waste products such as heavy metals, cyanides, or halogenated organics. In contrast, the biotransformation approach developed in this work is biocompatible with only unreacted glutamic acid, prenol, and boric acid as the byproducts of the synthetic step and only minimal use of non-aqueous solvents. Furthermore, this procedure is economically attractive, with reagents costing less than 10% of other kainic acid syntheses while also greatly simplifying the purification procedure and remaining scalable. In embodiments, this biotransformation strategy is employed to produce additional kainoids such as domoic acid, and novel kainic acid analogs from synthetic precursors.

Overall, combining genomic information with in vitro enzymatic characterization disclosed herein enabled discovery of the enzymes responsible for the biosynthesis of a seaweed natural product, and also to develop new methods to produce this important research tool.

### REFERENCES OF EXAMPLE 1

1. Higa, T. & Kuniyoshi, M. Toxins associated with medicinal and edible seaweeds. J. Toxicol.—Toxin Rev. 19, 119-137 (2000).
2. Nitta, I., Watase, H. & Tomiie, Y. Structure of kainic acid and its isomer, allokainic acid. Nature 181, 761-2 (1958).
3. Murakami, S., Takemoto, T. & Shimizu, Z. Studies on the effective principles of Digenea simplex Aq. I. Yakugaku Zasshi 73, 1026-1028 (1953).
4. Komiya, Y. & Kobayashi, A. Techniques applied in Japan for the control of Ascaris and hookworm infections—a review. Jpn. J. Med. Sci. Biol. 18, 1-17 (1965).
5. Lee, S. H., Kang, S. C., Ahn, J. H., Lee, J. W. & Rim, H. J. Santonin-kainic acid complex as a mass chemotherapeutic of Ascaris lumbricoides control in Korea. Korean J. Parasitol. 10, 79-85 (1972).
6. Tremblay, J.-F. Shortage of kainic acid hampers neuroscience research. Chem. Eng. News 78, 14-15 (2000).
7. Werner, P., Voigt, M., Keinanen, K., Wisden, W. & Seeburg, P. H. Cloning of a putative high-affinity kainate receptor expressed predominantly in hippocampal CA3 cells. Nature 351, 742-4 (1991).
8. Dingledine, R., Borges, K., Bowie, D. & Traynelis, S. F. The glutamate receptor ion channels. Pharmacol. Rev. 51, 7-61 (1999).
9. Hampson, D. R. & Manalo, J. L. The activation of glutamate receptors by kainic acid and domoic acid. Nat. Toxins 6, 153-8 (1998).
10. Lodge, D. The history of the pharmacology and cloning of ionotropic glutamate receptors and the development of idiosyncratic nomenclature. Neuropharmacology 56, 6-21 (2009).
11. Pulido, O. M. Domoic acid toxicologic pathology: A review. Mar. Drugs 6, 180-219 (2008).
12. Konno, K., Hashimoto, K., Ohfune, Y., Shirahama, H. & Matsumoto, T. Acromelic acids A and B. Potent neuroexcitatory amino acids isolated from Clitocybe acromelalga. J. Am. Chem. Soc. 110, 4807-4815 (1988).
13. Lelong, A., Hégaret, H., Soudant, P. & Bates, S. S. Pseudo-nitzschia (Bacillariophyceae) species, domoic acid and amnesic shellfish poisoning: revisiting previous paradigms. Phycologia 51, 168-216 (2012).
14. Mos, L. Domoic acid: a fascinating marine toxin. Environ. Toxicol. Pharmacol. 9, 79-85 (2001).
15. Grattan, L. M. et al. Repeated dietary exposure to low levels of domoic acid and problems with everyday memory: research to public health outreach. Toxins (Basel). 10, 103 (2018).
16. Wright, J. L. C. et al. Identification of domoic acid, a neuroexcitatory amino acid, in toxic mussels from eastern Prince Edward Island. Can. J. Chem. 67, 481-490 (1989).
17. Zhu, J., Zheng, X. Y., Zhang, H. L. & Luo, Q. Kainic acid-induced neurodegenerative model: Potentials and limitations. J. Biomed. Biotechnol. 2011, (2011).
18. Ben-Ari, Y., Lagowska, J., Tremblay, E. & Le Gal La Salle, G. A new model of focal status epilepticus: intra-amygdaloid application of kainic acid elicits repetitive secondarily generalized convulsive seizures. Brain Res. 163, 176-9 (1979).
19. Levesque, M. & Avoli, M. The kainic acid model of temporal lobe epilepsy. Neurosci. Biobehav. Rev. 37, 2887-2899 (2013).
20. Stathakis, C. I., Yioti, E. G. & Gallos, J. K. Total syntheses of (−)-α-kainic acid. European J. Org. Chem. 4661-4673 (2012). doi:10.1002/ejoc.201200243
21. Holdt, S. L. & Kraan, S. Bioactive compounds in seaweed: Functional food applications and legislation. J. Appl. Phycol. 23, 543-597 (2011).
22. Brunson, J. K. et al. Biosynthesis of the neurotoxin domoic acid in a bloom-forming diatom. Science 361, 1356-1358 (2018).
23. Michael, T. P. et al. High contiguity Arabidopsis thaliana genome assembly with a single nanopore flow cell. Nat. Commun. 9, 1-8 (2018).
24. Boycheva, S., Daviet, L., Wolfender, J. & Fitzpatrick, T. B. The rise of operon-like gene clusters in plants. Trends Plant Sci. 19, 447-459 (2014).
25. Schlapfer, P. et al. Genome-wide prediction of metabolic enzymes, pathways, and gene clusters in plants. Plant Physiol. 173, 2041-2059 (2017).
26. Brawley, S. H. et al. Insights into the red algae and eukaryotic evolution from the genome of Porphyra umbilicalis (Bangiophyceae, Rhodophyta). Proc. Natl. Acad. Sci. 114, E6361-E6370 (2017).
27. Matasci, N. et al. Data access for the 1,000 Plants (1KP) project. Gigascience 3, 17 (2014).
28. Saunders, G. W., Jackson, C. & Salomaki, E. D. Phylogenetic analyses of transcriptome data resolve familial assignments for genera of the red-algal Acrochaetiales-Palmariales Complex (Nemaliophycidae). Mol. Phylogenet. Evol. 119, 151-159 (2018).
29. Ramsey, U. P., Bird, C. J., Shacklock, P. F., Laycock, M. V. & Wright, J. L. C. Kainic acid and 1′-hydroxykainic acid from palmariales. Nat. Toxins 2, 286-292 (1994).
30. Monier, A. et al. Horizontal gene transfer of an entire metabolic pathway between a eukaryotic alga and its DNA virus. Genome Res. 19, 1441-9 (2009).
31. Miyasaki, M., Watanabe, H., Takano, T. & Morimoto, A. Studies on the components of Digenea simplex Ag. VII. Yakugaku Zasshi 76, 189-191 (1956).
32. Goldberg, O., Luini, A. & Teichberg, V. I. Lactones derived from kainic acid: novel selective antagonists of amino acid-induced Na+ fluxes in rat striatum slices. Neurosci. Lett. 23, 187-91 (1981).
33. Coudert, E., Acher, F. & Azerad, R. A convenient and efficient synthesis of (2S,4R)- and (2S,4S)-4-methylglutamic acid. Synthesis (Stuttg). 1997, 863-865 (1997).
34. Gupta, D., Summers, M. L. & Basu, C. Engineering an isoprenoid pathway in Escherichia coli for production of 2-methyl-3-buten-2-ol: A potential biofuel. Mol. Biotechnol. 56, 516-523 (2014).
35. Winkelblech, J., Fan, A. & Li, S. M. Prenyltransferases as key enzymes in primary and secondary metabolism. Appl. Microbiol. Biotechnol. 99, 7379-7397 (2015).
36. Martinez, S. & Hausinger, R. P. Catalytic mechanisms of Fe(II)- and 2-oxoglutarate-dependent oxygenases. J. Biol. Chem. 290, 20702-11 (2015).
37. Siitonen, V. et al. Divergent non-heme iron enzymes in the nogalamycin biosynthetic pathway. Proc. Natl. Acad. Sci. 113, 5251-5256 (2016).
38. Tang, M.-C., Zou, Y., Watanabe, K., Walsh, C. T. & Tang, Y. Oxidative Cyclization in Natural Product Biosynthesis. Chem. Rev. acs.chemrev.6b00478 (2016). doi:10.1021/acs.chemrev.6b00478
39. Sheldon, R. A. & Woodley, J. M. Role of biocatalysis in sustainable chemistry. Chem. Rev. 118, 801-838 (2018).
40. Zhang, M. et al. A short scalable route to (−)-α-kainic acid using Pt-catalyzed direct allylic amination. Chemistry 21, 3937-41 (2015).

### Example 2

### General Methods

Materials: all materials were purchased from Fisher Scientific, Alfa Aesar, Sigma-Aldrich, or Chem-Impex.

Bacterial growth selection: all bacteria transformed with pET28 were grown in the presence of the corresponding antibiotic kanamycin (50 mg/L).

NMR: NMR spectroscopic data was collected on a Bruker Avance III spectrometer (600 MHz) using either a 1.7 mm inverse detection triple resonance (H-C/N/D) cryoprobe or a 5 mm inverse detection triple resonance (H-C/N/D) cryoprobe or a JEOL spectrometer (500 MHz). All samples were dissolved in D20 and supplemented with 0.1% methanol to serve as a reference standard.

### Molecular Biology and Biochemical Methods

### DNA Extraction

Digenea simplex was collected from Onna, Okinawa Prefecture, Japan. The sample was dried by lyophilization and finely ground. Five hundred milligrams of powder was resuspended in 10 mL of CTAB buffer (3% CTAB, 1.4 M NaCl, 20 mM EDTA, 100 mM Tris-HCl pH 8.0, 0.2% polyvinylpolypyrrolidone, 0.2% β-mercaptoethanol, 1.5 mg/mL RNase A, and 0.2 mg/mL proteinase K) and incubated at 55° C. for 2 hours with gentle mixing every 15 minutes. The cell debris was removed by centrifugation at 14,000×g for 10 minutes at room temperature. The supernatant was transferred and 2 mL of 5 M potassium acetate pH 8.0 was added. The mixture was gently mixed and cooled on ice for 30 minutes then centrifuged at 14,000×g for 15 minutes at 4° C. The supernatant was removed and extracted with one equivalent of phenol:chloroform:isoamyl alcohol 25:24:1 saturated with 10 mM Tris, pH 8.0, 1 mM EDTA. The mixture was centrifuged at 12,000×g for 5 minutes at 4° C. The aqueous layer was transferred to a new tube and extracted with one equivalent of chloroform and centrifuged at before. The aqueous layer was transferred and one equivalent of cold isopropanol was added and carefully mixed. The mixture was centrifuged at 12,000×g for 15 minutes at 4° C. The supernatant was removed and the pellet was carefully washed with 75% ethanol. The pellet was dried to remove the ethanol and carefully resuspended in 250 μL of elution buffer (10 mM Tris-HCl pH 8.0). To further remove RNA, 300 μg of RNase A was added and incubated at room temperature for 1 hour prior to incubation at 4° C. overnight. The RNase A treated DNA was extracted with phenol:chloroform:isoamyl alcohol 25:24:1 saturated with 10 mM Tris, pH 8.0, 1 mM EDTA followed by chloroform extraction as before. Sodium acetate (pH 5.2) was added to a final concentration of 0.3 M followed by three equivalents of cold ethanol and carefully mixed. The mixture was incubated at −20° C. for 2 hours and centrifuged at 16,000×g for 20 minutes at 4° C. The pellet was washed with 75% ethanol and dried. The pellet was resuspended with 100 μL elution buffer and further purified by a PippinHT with >8,000 bp size selection. Palmaria palmata was collected from Wallace Cove Lighthouse, Bay of Fundy, New Brunswick, Canada and dried in silica gel. The sample was further dried by lyophilization and ground into a powder. DNA was extracted in a similar manner to the previous method, except 20 mL of CTAB buffer was used for 500 mg of P. palmata powder and RNase A was omitted. Because RNA was not completely digested after the overnight incubation an additional 100 μg of RNase was added and further incubated as before. DNA was directly used for sequencing without a PippinHT purification step.

### Genome Sequencing

The high molecular weight (HMW) D. simplex and P. palmata DNA was sequenced by running on an Oxford Nanopore MinION or GridION sequencer (Oxford Nanopore Technologies, Oxford, UK). A one-dimensional (ld) library was prepared with 1.2 μg DNA using the Ligation Sequencing Kit (SQK-LSK109, Oxford Nanopore Technologies, Oxford, UK). The libraries were loaded on a R9.4 flowcell and run for 48 hr resulting in 6.7 Gb of sequencing with a read length N50 of 7.2 kb and 4.7 Gb of sequencing with a read length N50 of 8 kb for D. simplex and P. palmata respectively. Since the HMW DNA generated both long reads (good distribution) and throughput, 1 μg of DNA was used to make Id library for the PromethION (Oxford Nanopore Technologies, Oxford, UK) for D. simplex. The resulting sequencing run produced 47 Gb of sequence with a read length N50 of 7 kb.

### Illumina Sequencing

NEBnext sequencing libraries were generated to polish the genome assemblies (New England Biolabs, Beverly, MA, USA). NEBnext sequencing libraries were created with 100 ng of DNA and quality controlled on a bioanalyzer. The resulting libraries were sequenced on an Illumina MiSeq 2×150 bp to check quality and quantity (Illumina, San Diego, CA). The libraries were then sequenced on an Illumina NovaSeq6000 2×150 bp run that resulted in 19 and 34 Gb of sequence for D. simplex and P. palmata respectively.

### Genome Assembly

Sequencing reads resulting from the PromethION (47 Gb) were assembled using a correctionless overlap-layout-consensus strategy was taken to assemble the genome [1]. The resulting raw reads in fastq format were aligned (overlap) with minimap [2] and an assembly graph (layout) was generated with miniasm [3]. The resulting graph was inspected using Bandage [4]. A consensus sequence was generated by mapping reads to the assembly with minimap, and then Racon [5] three times. Finally, the assembly was polished with pilon [6] three times using the Illumina pairedend 2×150 bp sequence; the Illumina reads were mapped to the consensus assembly using minimap2.

P. palmata Kab Cluster

The 4.7 Gb of ONT sequence generated on the GridION for P. palamta was used to search for long reads containing the ppkab cluster. The fastq file was transformed to a fasta file and made into a blast database. The DsKab genes were then used to search the long-read blast database by tblastn and a 44 kb read was identified with the ppkab cluster. A consensus sequence was generated by mapping reads to the single 44 kb read with minimap [2], and then Racon [5] two times. Polishing with Illumina reads was attempted on the 44 kb consensus read but the ppkab cluster was flanked by repeat elements with 60,000 and 100,000-fold coverage, which resulted in poor polishing. Therefore, only the kab cluster was polished with pilon [6] five times using the Illumina paired-end 2×150 bp sequence.

### PCR and Cloning

PCR of the Kab genes was performed with PrimeSTAR HS DNA Polymerase (Takara) according to the manufacturer's instructions using either genomic DNA or codon optimized synthetic genes as a template. pET28-MBP (Maltose Binding Protein)-TEV and pET28 vectors were amplified using PrimeSTAR MAX DNA Polymerase (Takara). PCR products were purified by agarose gel extraction and assembled into expression vectors using NEBuilder HiFi DNA Assembly mix and transformed into E. coli DH5a cells. Construction of vectors was confirmed by Sanger sequencing. The pET28-MBP-TEV vector produced proteins with a His₆-MBP-His₆ N-terminal tag and the pET28 vector produced proteins with a His₆ N-terminal tag. See Table S1 and S2 for a list of primers and synthetic genes respectively.

TABLE S1
primers used in this example.

| Name | Sequence | SEQ ID NO: |
|---|---|---|
| DsKabA p28 For | GGTGCCGCGCGGCAGCCATATGAGTATTAC | 21 |
| | CACTATGAAAGGAGTTACATC | |
| DsKabA p28 Rev | GGTGGTGGTGGTGCTCGAGTTAATTTGTCGC | 22 |
| | CTTGAGTTTCTTAAAG | |
| DsKabC p28 For | GGTGCCGCGCGGCAGCCATATGACAGTAAG | 23 |
| | TAAGTACAACGGTGTTG | |
| DsKabC p28 Rev | GGTGGTGGTGGTGCTCGAGTTAAAGGTAGT | 24 |
| | ATCCTTCATGAAATTTG | |
| GfKabC MBP For | CTGTACTTCCAATCCGGATCCATGACTTCTT | 25 |
| | TCGATTTCGTCGCTAAGACTTTC | |
| GfKabC MBP Rev | GGTGGTGGTGGTGCTCGAGTTAGACGTAGT | 26 |
| | ACCCCGAGTGAAACTTTGC | |
| PpKabC MBP For | CTGTACTTCCAATCCGGATCCATGCCTGTGT | 27 |
| | ATAATGGTACGTCACAAGC | |
| PpKabC MBP Rev | GGTGGTGGTGGTGCTCGAGTTACAGGTAGT | 28 |
| | AACCTTCATGGAATTTGTTCTTG | |
| ReKabC MBP For | CTGTACTTCCAATCCGGATCCATGGCGGTGT | 29 |
| | ACAATGGCGTCTCGCAGG | |
| ReKabC MBP Rev | GGTGGTGGTGGTGCTCGAGCTATAGATAAT | 30 |
| | AGCCCTCGTGGAACTTATTTTTCACG | |
| pET28 Up | CATATGGCTGCCGCGCGGCACC | 31 |
| pET28 Down | CTCGAGCACCACCACCACCACCACTGAG | 32 |

TABLE S2
Synthesized genes used in this study were purchased as codon
optimized gBlocks from IDT.

| | | SEQ ID |
|---|---|---|
| Name | Sequence | NO: |
| PpKabC | ATGCCTGTGTATAATGGTACGTCACAAG | 33 |
| | CATTCAATTTTGCGCCGCTGCACCCAGATAGTT | |
| | TGAATTGGTGCCCAAAGTCGAGTCTTCCTCCAG | |
| | AAATTCCTGTAGTGGATCTGTCAAAACTTAATT | |
| | CTGAAGCCGAGCTGGCACAATTTCTTGTGGACA | |
| | TTCGTAAGAGCGGGTTGTTTTATGTAGTCAATC | |
| | ATGGTGTCCCGGAAGAACTTTCTATTGGGTGTT | |
| | ACAATCGCTTCCGTCAATTCTGTAACATTCCTG | |
| | AGGACCAACGTGCGGAATACTCGACCGACCAT | |
| | CATTTTGTAAACGGGGGCTACATGCCGTATAAG | |
| | AGTTCCAGCATCGGCAAGGCAAATAAAGGTAA | |
| | ATCTCAAAAGGACTTTGTGGTTAAATACTTCTG | |
| | GCGTGGACCGCGCGTTGAGAACCGTAGTCCGG | |
| | ATGCTGATTTCAAGAGCTATCACGACGAATACC | |
| | ACCGTCGCACAGCTGATATCGCTAACAGCGTGA | |
| | TTACTAAAATTCTTCAGGCCCTGACGACGCGTT | |
| | TTCCTGACTTTGACCCTGCTGAATATAAAGATA | |
| | ACATTAATTCCCATCATATGTTCTTCTCGAACCG | |
| | CCTTTACCCAGACAACGAACCTGAGAAAGGCG | |
| | AGAATGTAGAATACCGTTTAGTACCGCACCGTG | |
| | ACCTGAGCTTCGTGACCCTTGCTCACCAAATCC | |
| | CGGCGGACAACGGTTACCAAGGCCTTTTCGTTT | |
| | TTACCGGCGATGGGAAGAAGGTTTGCGTGCCTC | |
| | CGATTCGTAACAGCTATCTTGTCTTCATGGGAC | |
| | AGGCAATGTCGTATCTGACGAATAAGTACTTAC | |
| | CTGCAGGGTTGCATGGCGTGGATTTTCCTGAAA | |
| | AGAACTGTTTCGAGGGGTCTGAACGCTCCTCTC | |
| | TTATCTCATTTTACGAACCTCACGATCATATGAT | |
| | GCCCAGCAAAGCACTTACACCTAAGGATGATG | |
| | AGATCTTCGATCGTGGTTGTTCGTTCTTTGATGA | |
| | CATCGGCGTGGACAAAACAGGGACGACCTATA | |
| | TGTATGTCAAGAACAAATTCCATGAAGGTTACT | |
| | ACCTGTAAG | |
| GfKabC | ATGACTTCTTTCGATTTCGTCGCTAAGAC | 34 |
| | TTTCGATTTTGCTCCCGTCAAAGCTGACGAGTT | |
| | GAAGTGGTATCCACGTTCCGCGTTACCACCAGA | |
| | AATTCCTGTAATTGATTTGAACAACGTCAATAC | |
| | GGAGGAGGAGCTTGCACAGTTCCTGGTGGACA | |
| | TCCGCAAGTCTGGACTTTTCTACGTTGTCAACC | |
| | ACGATATCCCGGAGGAAATCTCAATCCAGACGT | |
| | ACAACGAATTTCGTGAGTTTTGTAAATTGCCAG | |
| | AAGAGGCCCGCCAAAAATACAATACTGATCGC | |
| | TGCTTCTTCAATGGGGGATATGTTCCATTCAAA | |
| | GCCACGAGCATTAATGGGGGTAATAAGAACAA | |
| | ACAGCAACGCGACTTTGTCGTGAAGTTTTTCTG | |
| | GCGCGGCCCACACGTGGTCAATCGTTCCCCCAA | |
| | TGAACGCTTTGCCAAATGGCACCATCAGTATCA | |
| | TACACGCACTGCTGAATTAGGTGAAAAGGTCAT | |
| | GACCACAATCGCAAAAGCTTTGAAGACGCGCTT | |
| | TCCTGACTTCGACCCGGCCGAACTGGAGGACAA | |
| | TGTGAACCCTCACAACATGATTTTTTCTAACCG | |
| | CATTTATCCGGAATTCCCACCCAGTGAAGGGGA | |
| | GGATGCTGAGTATCGCCTGACCCCCCACCGTGA | |
| | TATTTCCTACATTACCCTTGTGAATCAGACTCCT | |
| | GCCAATAACGGATTCAAAGCATTATTTATCGTA | |
| | ACCGGGGACGGCGAACGCGTTTACGTCCCTCCT | |
| | ATCCGCAACTCTTACTTGGTATTCATCGGGCAG | |
| | AGCTTGAGCTACCTTACCAATAAATACCTTCCA | |
| | GCGGCACTTCACGGGGTCGCTTTTCCGGACGGC | |
| | AATCTTGAGGGTTGCGAGCGTGCCTCACTGGTG | |
| | TCTTTCTATGAACCGTATGATCGCATGGTCCCA | |
| | TCGAAAAATATCAAACCAACGGCAGAAGAGAT | |
| | CGCTCCAAAATCCTGCTCCTTCTATGATTCGATT | |
| | GGGTGTGACAAGACAGGAACTACCTTTACTGAT | |
| | GTTCGTGCAAAGTTTCACTCGGGGTACTACGTC | |
| | TAA | |

Digenea simplex Kainic Acid Extraction

Twenty milligrams of D. simplex were finely ground and soaked in 300 mL of distilled water for 2 h at 55° C. The sample was filtered and the flow through was analyzed by an Agilent Technologies 1200 Series system with a diode-array detector coupled to an Agilent Technologies 6530 accurate-mass Q-TOF LCMS using the following method on a Synergi Polar-PP 4μ. 250×4.6 mm column at 0.75 mL/min: 0% B (4.5 min), 0 to 5% B (0.5 min), 5 to 26% B (9 min), 26 to 80% B (9 min), 80 to 100% B (1 min), 100% B (1.5 min), 100 to 0% B (2.5 min), 0% B (2 min), wherein A=0.10% aqueous formic acid, and B=0.10% formic acid in acetonitrile. HRMS (ESI) calculated for prekainic acid (C₁₀H₁₆NO₄⁻) 214.1085, found 214.1078 (M-H)⁻; kainic acid (C₁₀H₁₄NO₄⁻) 212.0928, found 212.0936 (M-H)⁻; and kainic acid lactone (C₁₀H₁₄NO₄⁻) 212.0928, found 212.0922 (M-H)⁻.

### Protein Purification

E. coli BL-21 co-transformed with the protein expression plasmid were shaken 37° C. in 1 L of terrific broth to an OD₆₀₀˜0.8 and then cooled at 18° C. for 1 h before addition of 0.5 mM IPTG for pET28 vectors or 0.2 mM IPTG for MBP vectors. The flasks were shaken overnight and harvested by centrifugation the following morning to remove the media. The cells were resuspended in 25 mL of suspension buffer (500 mM NaCl, 20 mM Tris pH 8.0, and 10% glycerol) and lysed by sonication with a Qsonica 6 mm tip at 40% amplitude for 12 cycles of 15 seconds on and 45 seconds off. The cell lysate was centrifuged at 14,000×g for 25 minutes to removed insoluble debris. The cleared lysate was loaded at 2 mL/min onto a 5 mL HisTrap FF column (GE Healthcare Life Sciences) that was pre-equilibrated with buffer A (1 M NaCl, 30 mM imidazole, and 20 mM Tris pH 8.0) using an ÄKTA FPLC (GE Healthcare Life Sciences). After loading, the column was washed with 40 mL of buffer A. Protein was eluted using a linear gradient of 0-100% buffer B (1 M NaCl, 250 mM imidazole, and 20 mM Tris pH 8.0) over 40 mL and 5 mL fractions were collected. The fractions were analyzed by SDS-PAGE and fractions with at least 90% purity were combined. For the KabC proteins, 2 mM EDTA was added to remove any metals bound in the active site. If the His₆ tag was to be removed, 60 units of thrombin were added and incubated overnight at 4° C. Complete cutting was confirmed by SDS-PAGE. Protein was concentrated using Amicon Ultra Centrifugal Filters and further purified by gel filtration using a either a HiLoad 16/60 Superdex 75 or 200 prep grade gel filtration column (GE Healthcare Life Sciences) preequilibrated with 300 mM KCl and 50 mM HEPES pH 8.0. If the protein was to be frozen following gel filtration, 10% glycerol was also added.

### Enzymatic Activity Assays

To test DsKabA activity and substrate specificity, a 50 μL reaction containing 5 mM MgCl₂, 5 mM amino acid (L-Glu, D-Glu, L-Gln, L-Asp, L-Asn, or Gly), 1 mM prenyl donor (DMAPP or GPP), and 20 μM DsKabA in a buffer of 300 mM KCl and 50 mM HEPES pH 8.0 was incubated at 23° C. for 5 h. One equivalent of 0.1% formic acid in H₂O was added and filtered with a VWR PES 3 k 500 μL spin column. The flow through was analyzed by an Agilent Technologies 1200 Series system with a diode-array detector coupled to an Agilent Technologies 6530 accurate-mass Q-TOF LCMS using the following method on a Synergi Polar-RP 4μ 250×4.6 mm column at 0.75 mL/min: 0% B (4.5 min), 0 to 5% B (0.5 min), 5 to 26% B (9 min), 26 to 80% B (9 min), 80 to 100% B (1 min), 100% B (1.5 min), 100 to 0% B (2.5 min), 0% B (2 min), wherein A=0.1% aqueous formic acid, and B=0.1% formic acid in acetonitrile. For KabC activity and substrate specificity assays, 50 μL reactions containing 5 mM αKG, 1 mM L-ascorbic acid, 1 mM substrate (prekainic acid, NGG, or cNGG) 30 μM ferrous sulfate, and 20 μM KabC in a buffer of 300 mM KCl and 50 mM HEPES pH 8.0 were incubated at 23° C. for 5 h. One equivalent of 0.1% formic acid in H₂O was added and filtered as before. The flow through was analyzed using the same methodology as DsKabA.

### DsKabA Prekainic Acid Reaction Scaleup and Purification

A 5 mL reaction of 30 μM His₆-DsKabA, 5 mM L-Glu, 10 mM MgCl₂, and 5 mM DMAPP in a buffer of 300 mM KCl and 50 mM HEPES pH 8.0 was incubated at 22° C. for 18 h. The protein was separated from the reaction using a 30 kDa Amicon Ultra Centrifugal Filter and washed to recover all small molecule products. The collected flow through was lyophilized and resuspended in 0.1% aqueous formic acid before being purified by preparative RP-HPLC (Agilent PrepStar and ProStar 410 HPLC) using a Phenomenex Luna 5μ C18(2), 100×21.2 mm column at a flow rate of 10 mL/min with the following method: 2% B (5 min), 2 to 20% B (10 min), 20 to 95% B (1 min), 95% B (4 min), 95 to 2% B (2 min), 2% B (3 min), wherein A=0.1% aqueous formic acid, and B=0.1% formic acid in acetonitrile. Absorbance at 210 nm was monitored and the major peak was collected. Acetonitrile was removed by rotary evaporation before lyophilization to afford prekainic acid (3.7 mg, 56% yield) as a white powder. 1H NMR (599 MHz, D₂O) δ 5.22 (t, J=7.3 Hz, 1H), 3.64 (t, J=6.7 Hz, 2H), 3.61 (t, J=6.4 Hz, 1H), 2.55-2.42 (m, 2H), 2.12 (ddt, J=13.7, 7.3, 7.3 Hz, 1H), 2.03 (dtd, J=14.3, 7.3, 6.9 Hz, 1H), 1.76 (s, 3H), 1.68 (s, 3H). ¹³C NMR (151 MHz, D₂O): δ 177.6, 173.3, 144.8, 113.0, 60.2, 44.5, 30.5, 25.4, 25.4, 17.7; HRMS (ESI) calculated for C₁₀H₁₆NO₄⁻ 214.1085, found 214.1088 (M-H)⁻.

### DsKabC Kainic Acid Reaction Scaleup and Purification

A 7.5 mL reaction of 30 μM His₆-DsKabC, 5 mM prekainic acid, 10 mM αKG, 2 mM L-ascorbate, and 50 μM ferrous sulfate in a buffer of 300 mM KCl and 50 mM HEPES pH 8.0 was incubated at 22° C. for 18 h. The protein was separated from the reaction using a 30 kDa Amicon Ultra Centrifugal Filter and washed to recover all small molecule products. The collected flow through was lyophilized and resuspended in 0.1% aqueous formic acid before being purified by preparative RP-HPLC (Agilent PrepStar and ProStar 410 HPLC) using a Phenomenex Luna 5μ C18(2), 100×21.2 mm column at a flow rate of 10 mL/min with the following method: 2% B (5 min), 2 to 20% B (10 min), 20 to 95% B (1 min), 95% B (4 min), 95 to 2% B (2 min), 2% B (3 min), wherein A=0.1% aqueous formic acid, and B=0.1% formic acid in acetonitrile. Absorbance at 210 nm was monitored and the major peak was collected. Acetonitrile was removed by rotary evaporation before lyophilization to afford kainic acid (4.5 mg, 46% isolated yield) as a white powder. ¹H NMR (599 MHz, D₂O) δ 5.01 (s, 1H), 4.73 (s, 1H), 4.07 (d, J=3.0 Hz, 1H), 3.60 (dd, J=11.9, 7.3 Hz, 1H), 3.40 (dd, J=11.5, 11.5 Hz, 1H), 3.09-3.02 (m, 1H), 3.02-2.95 (m, 1H), 2.43 (dd, J=16.6, 6.4 Hz, 1H), 2.34 (dd, J=16.6, 8.4 Hz, 1H), 1.73 (s, 3H). ¹³C NMR (151 MHz, D₂O): δ 176.9, 173.6, 140.3, 113.6, 66.5, 46.8, 46.2, 40.8, 33.5, 22.1; HRMS (ESI) calculated for C₁₀H₁₄NO₄− 212.0928, found 212.0929 (M-H)⁻.

### GfKabC Kainic Acid Lactone Reaction Scaleup and Purification

MBP-GfKabC was buffer exchanged into 300 mM KCl, 50 mM HEPES pH 8.0 to remove glycerol. Six 1 mL reactions of GfKabC were set up as follows: 8 mM αKG, 5 mM prekainic acid, 1 mM Lascorbic acid, 50 μM ferrous sulfate, 50 μM MBP-GfKabC in buffer containing 300 mM KCl, 50 mM HEPES pH 8.0. The reaction was incubated at 22° C. for 30 hours and the protein was removed with 30 kDa Amicon Ultra Centrifugal Filter. The protein was washed with distilled water and the flow throughs were collected and lyophilized. The dried material was resuspended in 500 μL of 0.1% aqueous formic acid and purified by semipreparative HPLC (Agilent 1200 Infinity HPLC) using a Synergi Polar-RP 4μ 250×10.0 mm column with the following method: 0% B (18 min, 2 mL/min), 0 to 95% B (2 min, 3 mL/min), 95% B (3 min, 3 mL/min), 95 to 0% B (4 min, 3 mL/min), 0% B (3 min, 3 mL/min), wherein A=0.1% aqueous formic acid, and B=0.1% formic acid in acetonitrile. The major peak (˜9.5 min retention time) was collected and lyophilized to afford kainic acid lactone (3.3 mg, 26% isolated yield) as a white powder. ¹H NMR (600 MHz, D₂O) δ 3.97 (d, J=3.9 Hz, 1H), 3.73 (dd, J=12.2, 8.2 Hz, 1H), 3.24 (dd, J=11.9, 11.9 Hz, 1H), 3.22-3.16 (m, 1H), 3.15 (dd, J=18.0, 9.8 Hz, 1H), 2.79 (ddd, J=11.5, 8.1, 8.1 Hz, 1H), 2.55 (dd, J=17.5, 3.9 Hz, 1H), 1.48 (s, 3H), 1.39 (s, 3H). ¹³C NMR (151 MHz, D₂O): δ 174.3, 172.9, 82.8, 68.3, 45.3, 42.8, 36.5, 30.9, 27.9, 26.6; HRMS (ESI) calculated for C₁₀H₁₄NO₄− 212.0928, found 212.0930 (M-H)⁻

### Kainic Acid Biotransformation and Purification

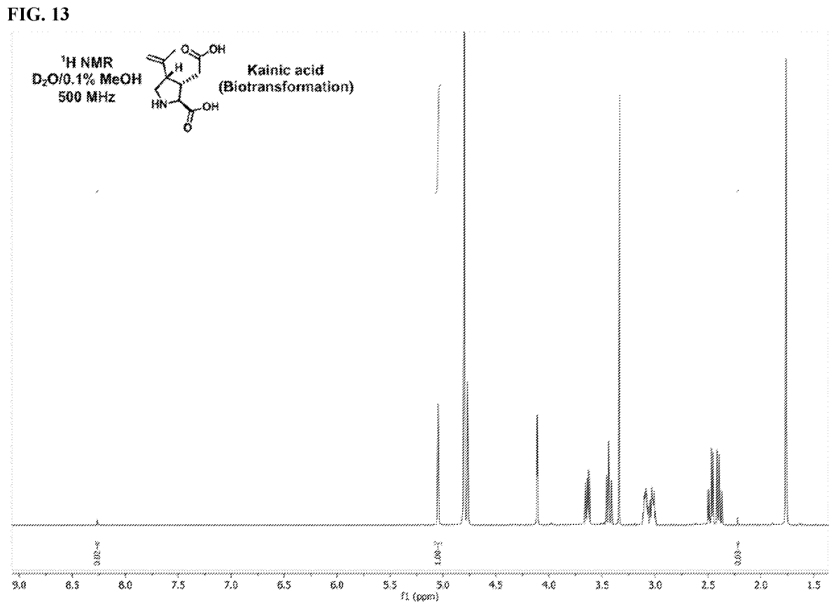

E. coli BL-21 transformed with the pET28 DsKabC vector were grown overnight in 20 mL of lysogeny broth. The culture was pelleted to remove the media and added to 1 L of M9 minimal media supplemented with 100 μM CaCl2) in a 2.8 L baffled flask. The flasks were placed in a shaking incubator (200 rpm, 37° C.) and the cells were grown to an OD₆₀₀ of ˜1.3. The flasks were cooled at 18° C. for 45 minutes followed by the addition of 0.5 mM IPTG. Fifteen minutes after IPTG was added, 4 g of glucose and the crude prekainic acid synthetic reaction product was added with continuous shaking incubation at 18° C. Additional glucose (4 g) was added after 16 h. Time points were taken and analyzed for the presence of kainic acid using the LCMS methodology described in the kainic acid fermentation protocol. After 40 h, the cultures were centrifuged to remove cells and the media was collected. Thirty grams of activated carbon (Darco G-60) per 1 L of media was washed with distilled water and added to the cell free media. The activated carbon was stirred with the media for 30 min before filtering to remove the spent media. The activated carbon was washed with distilled water twice and then rapidly stirred in 300 mL of 80% acetic acid for 15 min to elute the kainic acid. Three additional 300 mL 80% acetic acid elutions were used to maximize kainic acid recovery. The eluent was dried by rotary evaporation to afford a reddish oil which was further purified by preparative RP-HPLC (Agilent PrepStar and ProStar 410 HPLC) using a Phenomenex Luna 5μ C18(2), 100×21.2 mm column at a flow rate of 10 mL/min with the following method: 2% B (5 min), 2 to 20% B (10 min), 20 to 95% B (1 min), 95% B (4 min), 95 to 2% B (2 min), 2% B (3 min), wherein A=0.1% aqueous formic acid, and B=0.1% formic acid in acetonitrile. Absorbance at 210 nm was monitored and the major peak was collected. The kainic acid containing fractions were combined and the volume was reduced in vacuo before lyophilization. Using this method, 6 L of culture were used to afford 1.07 g of kainic acid (32% overall isolated yield) as a white powder. NMR (FIG. 13) indicates >95% purity with the major contaminants consistent with acetic acid (˜2%) and formic acid (˜2%).

### Synthetic Methods

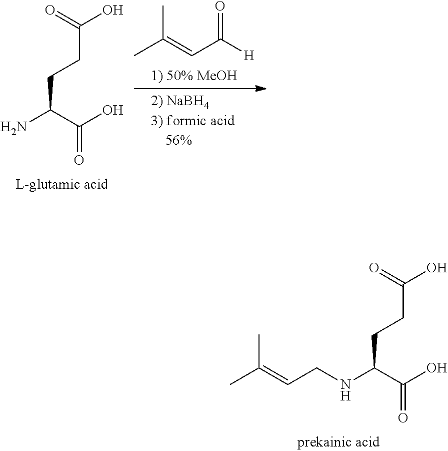

### Prekainic Acid Chemical Synthesis

This molecule was synthesized using a modified published procedure [10]. An aqueous solution of L-glutamic acid (0.147 g, 1 mmol) and crushed NaOH (0.083 g, 2.1 mmol) in 2.5 mL of H₂O was stirred at RT. MeOH (2.5 mL) was added. 3-methyl-2-butenal (0.135 mL, 1.4 mmol) was added and the reaction was immediately cooled in an ice bath while stirring. NaBH₄ (0.048 g, 1.3 mmol) was added and stirred for 20 minutes. The reaction was brought to room temperature over 20 minutes and was then acidified to ˜pH 4.0 with formic acid. The methanol was removed in vacuo and the reaction was purified by injection onto preparative RP-HPLC (Agilent 1260 Infinity HPLC) using a Phenomenex Luna 5μ C18(2), 100×21.2 mm column at a flow rate of 10 mL/min with the following method: 2% B (5 min), 2 to 20% B (10 min), 20 to 95% B (1 min), 95% B (4 min), 95 to 2% B (2 min), 2% B (3 min), wherein A=0.1% aqueous formic acid, and B=0.1% formic acid in acetonitrile. Absorbance at 210 nm was monitored and the major peak was collected. Acetonitrile was removed by rotary evaporation before lyophilization to afford prekainic acid (147 mg, 56% isolated yield) as a white powder. ¹H NMR (599 MHz, D₂O) δ 5.22 (t, J=7.8 Hz, 1H), 3.64 (t, J=7.9, 6.4 Hz, 2H), 3.61 (dd, J=7.7, 5.5 Hz, 1H), 2.56-2.42 (m, 2H), 2.13 (ddt, J=13.8, 7.0, 7.0 Hz, 1H), 2.03 (dtd, J=14.4, 7.3, 6.7 Hz, 1H), 1.76 (s, 3H), 1.68 (s, 3H). ¹³C NMR (151 MHz, D₂O): δ 177.5, 173.8, 145.3, 113.2, 60.4, 44.5, 30.1, 25.5, 25.4, 17.9; HRMS (ESI) calculated for C₁₀H₁₆NO₄⁻ 214.1085, found 214.1088 (M-H)⁻.

### Prekainic Acid Chemical Synthesis for Biotransformation

An aqueous solution of L-glutamic acid (1.18 g, 8.0 mmol) and crushed NaOH (0.672 g, 16.8 mmol) in 20 mL of H₂O was stirred at room temperature. MeOH (20 mL) was slowly added. 3-methyl-2-butenal (1.08 mL, 11.2 mmol) was added and the reaction was immediately cooled in an ice bath while stirring. NaBH₄ (0.393 g, 10.4 mmol) was added and stirred for 20 minutes. The reaction was brought to room temperature over 20 minutes and was then acidified with formic acid to ˜pH 4.0. The methanol was removed in vacuo. The crude reaction product was neutralized with NaOH to ˜pH 8.0 and ⅓ of the reaction was added to a 1 L biotransformation.

### DMAPP Chemical Synthesis

This synthesis was adapted from a literature procedure [11]. To a solution of tris (tetrabutylammonium) hydrogen pyrophosphate trihydrate [11] (3.00 g, 3.32 mmol) in dry acetonitrile (15 mL) at −35° C. was added prenyl bromide (0.19 mL, 0.25 g, 1.66 mmol) over 2 minutes. The reaction mixture was stirred at −35° C. for 10 minutes, then warmed to room temperature and stirred for an additional 2 hours. The solvent was removed in vacuo and the crude residue was resuspended in minimal ion exchange buffer (25 mM aqueous NH₄HCO₃, 2% isopropanol) and passed through a column of DOWEX AG50W-X8 (NH₄+form, 40 mL (˜70 milliequivalents)). The first two column volumes (80 mL) were collected and lyophilized to generate an off-white solid. This crude mixture was further purified via resuspension in 50 mM aqueous NH₄HCO₃ (2 mL), addition of 1:1 acetonitrile:isopropanol (8 mL) with thorough mixing, centrifugation (2000×g, 5 minutes) and pipetting the resultant supernatant into a new flask, repeating this procedure 5 times in total. The pooled supernatants were concentrated in vacuo then further purified by cellulose chromatography prepared in an analogous way to that previously described [11]. Purified DMAPP was eluted using a gradient of (1:2:1) to (2:2:1) water:acetonitrile:isopropanol (50 mM NH₄HCO₃). Pooled fractions were concentrated in vacuo and lyophilized, generating trisammonium DMAPP (0.452 g, 91%) as a white powder. ¹H NMR (500 MHz, D20) δ 5.31 (t, J=7.2 Hz, 1H), 4.31 (dd, J=7.0, 6.8 Hz, 2H), 1.62 (s, 3H), 1.58 (s, 3H); LRMS (ESI) calculated for C₅H₁₁O₇P₂− 245.00, found 245.02 (M-H)⁻.

### REFERENCES OF EXAMPLE 2

1. T. P. Michael, F. Jupe, F. Bemm, S. T. Motley, J. P. Sandoval, C. Lanz, O. Loudet, D. Weigel, J. R. Ecker, Nat. Commun. 2018, 9, 541.
2. H. Li, Bioinformatics 2018, 34, 3094-100.
3. H. Li, Bioinformatics 2016, 32, 2103-10.
4. R. R. Wick, M. B. Schultz, J. Zobel, K. E. Holt, Bioinformatics 2015, 31, 3350-2.
5. R. Vaser, I. Sovid, N. Nagarajan, M. Sikid, Genome Res. 2017, 27, 737-46.
6. B. J. Walker, T. Abeel, T. Shea, M. Priest, A. Abouelliel, S. Sakthikumar, C. A. Cuomo, Q. Zeng, J. Wortman, S. K. Young, et al., PLoS One 2014, 9, e112963.
7. K. Katoh, J. Rozewicki, K. D. Yamada, Brief Bioinform. 2017, 1-7.
8. W. P. Maddison, D. R. Maddison, “Mesquite: a modular system for evolutionary analysis,” 2018.
9. D. Silvestro, I. Michalak, Org. Divers. Evol. 2012, 12, 335-7.
10. J. K. Brunson, S. M. K. McKinnie, J. R. Chekan, J. P. McCrow, Z. D. Miles, E. M. Bertrand, V. A. Bielinski, H. Luhavaya, M. Obornik, G. J. Smith, et al., Science 2018, 361, 1356-8.
11. A. B. Woodside, Z. Huang, C. D. Poulter, Org. Synth. 1988, 66, 211-6.
12. G. W. Saunders, C. Jackson, E. D. Salomaki, Mol. Phylogenet. Evol. 2018, 119, 151-9.
13. N. Matasci, L. Hung, Z. Yan, E. J. Carpenter, N. J. Wickett, S. Mirarab, N. Nguyen, T. Warnow, S. Ayyampalayam, M. Barker, et al., Gigascience 2014, 3, 17.
14. J. Janouskovec, A. Horik, M. Obornik, J. Lukes, P. J. Keeling, Proc. Natl. Acad. Sci. U.S.A 2010, 107, 10949-54.
15. C. Bowler, A. E. Allen, J. H. Badger, J. Grimwood, K. Jabbari, A. Kuo, U. maheswari, C. Martens, F. Maumus, R. P. Otillar, et al., Nature 2008, 456, 239-44.
16. N. P. Dunham, W.-C. Chang, A. J. Mitchell, R. J. Martinie, B. Zhang, J. A. Bergman, L. J. Rajakovich, B. Wang, A. Silakov, C. Krebs, et al., J. Am. Chem. Soc. 2018, 140, 7116-26.
17. R. Calaf, A. Barlatier, D. Gargon, G. Balansard, M. Pellegrini, J. Reynaud, J Appl. Phycol. 1989, 1, 257-66.
18. O. Goldberg, A. Luini, V. I. Teichberg, J. Med. Chem. 1983, 26, 39-42.

### Example 3

### Kainic Acid Biotransformation and Purification

First, E. coli BL-21 cells previously transformed with the pET28 DsKabC vector were inoculated into 20 mL of lysogeny broth supplemented with 50 mg/L kanamycin and grow overnight into a shaking incubator at 37° C. The E. coli culture was then pelleted to remove the media and add to 1 L of M9 minimal media (see M9 minimal media recipe). Following addition of the M9 minimal media, the flask was placed in a shaking incubator (200 rpm, 37° C.) and the cells grown to an OD₆₀₀ of ˜1.3. (about 7 hours). Once the OD was reached, the flasks were cooled at 18° C. for 45 minutes and IPTG (isopropyl β-D-1-hiogalactopyranoside) was added to a final concentration of 0.5 mM. After 15 minutes, 4 g of filter sterilized glucose and ⅓ of the crude prekainic acid synthetic reaction product were added (see synthetic method), and incubation continued at 18° C. with shaking. Additional 4 g of filter sterilized glucose were added after 16 h of incubation. After 40 h of incubation, the culture was centrifuged to remove cells and to collect the media. Cells were bleached and properly disposed of. Then, 40 g of activated carbon were washed (Darco G-60) per 1 L of media with distilled water and filter dry using Buchner funnel, filter paper, and a vacuum flask. The washed activated carbon was added to the media and the solution was then stirred for 30 min. Filtering was then realized using the Buchner funnel setup to remove the spent media, and the activated carbon was washed with distilled water twice. The activated carbon was stirred in 300 mL of 80% acetic acid for 15 min to elute the kainic acid and collection was realized by filtration. An additional 300 mL 80% acetic acid elution could be used to maximize kainic acid recovery. The eluent was loaded onto 20 g of Dowex 50wx8 resin (H⁺ form), and the resin washed with water subsequently. The kainic acid was eluted with 2 M ammonium hydroxide in 50% methanol and 50% water solution. The kainic acid containing fractions was combined and the volume reduced by rotary evaporation. Freezing and lyophilization steps were used to afford a light orange powder. The powder was then dissolved in a minimal volume of hot 1:1 ethanol to water solution (approximately 16 mL per 1 g of crude powder), and the solution acidified with formic acid to a final concentration of 1% v/v. Crystallization was initiated and then allowed to continue by leaving the solution at room temperature, then 4° C. and finally −20° C. The resulting crystals were collected and then washed with cold isopropanol. A recrystallization step could be performed to improve purity.

### M9 Minimal Media

To prepare the M9 minimal media, the solution in the table below was prepared the night before inoculation, in a 2.8 L baffled flask.

| Component | Formula | Amount | |
|---|---|---|---|
| Sodium phosphate | Na₂HPO₄*7H₂O | 11.2 | grams |
| dibasic heptahydrate | | | |
| Potassium phosphate | KH₂PO₄ | 3 | grams |
| monobasic | | | |
| Ammonium chloride | NH₄Cl | 1 | gram |
| Sodium chloride | NaCl | 0.5 | grams |
| Milli-Q water | | 1 | L |

On the day of inoculation, the stock solutions shown in the table below were added the autoclaved solution contained in the 2.8 L baffled flask.

| Solution | Amount | | Notes |
|---|---|---|---|
| 25% glucose | 16 | mL | Filter sterilized and stock |
| | | | kept at 4° C. or −20° C. |
| 1M MgSO₄ | 1 | mL | Filter sterilized and can |
| | | | stock kept at room temp |
| 77 mg/mL FeSO₄ *7H₂O | 0.1 | mL | Prepared fresh |
| 0.5% thiamine | 0.1 | mL | Filter sterilized and |
| | | | stock could be freezed |
| 100 mM CaCl₂ | 1 | mL | Filter sterilized and |
| | | | can stock kept at room temp |
| 50 mg/mL Kanamycin | 1 | mL | Filter sterilized and |
| | | | stock could be freezed |

### Prekainic Acid Chemical Synthesis for Biotransformation

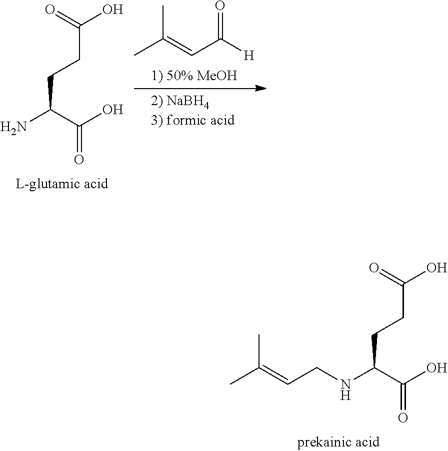

L-glutamic acid (1.18 g, 8.0 mmol) and crushed NaOH (0.672 g, 16.8 mmol) were added to 20 mL of H₂O and stirred at room temperature, to which 20 mL of methanol were slowly added. Then, 3-methyl-2-butenal (1.08 mL, 11.2 mmol) was added and the reaction was immediately cooled in an ice bath while stirring. After the 3-methyl-2-butenal step, argon was added to the headspace of the bottle to prevent oxidation overtime. Sodium borohydride (0.393 g, 10.4 mmol) was added and stirred for 20 minutes. The reaction was then removed from the ice water bath and stirring continued at room temperature for 20 minutes. The reaction was quenched by adding formic acid dropwise until bubbling stopped and the pH reached ˜4.0. Methanol was removed by rotary evaporation, and the concentrate neutralized to ˜ pH 8.0 with NaOH. Finally, ⅓ of this reaction was added to 1 L of the biotransformation.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

### Exemplary Sequences:

SEQ ID NOs: 1-5, respectively:
DsKabC (Digenea simplex):
MTVSKYNGVASTFNGVARRFDFAPLEADKLNWYPRSALPPEIPAIDLNKVNTEEELAQF
LVDIRKSGLFYIVDHGIPEEISIGCYNAFREFCNLPEEAREKYNTDESFKSGGYVPFKGTSI
GGGNLFERQKDFVVKFFWRGPSVVNRSPNDRFAEFHDEHHRKTAELAEKIITTILKALK
TRFPEFHPDELKDNINVRNMFFSNRIYPEAPPDDGEKADYRLVPHRDLSFITLANQVPAN
NGFKGLFILTGDGEKIPVPPIRNSYLVFIGQGLSYLTNKYLPAALHGVDFPDNTNFEGSER
ASLISFYEPNDYMMPSKNINPLPEEIFEKSCTFYDDVGVGRAGTTYNYVRYKFHEGYYL
PpKabC (Palmaria palmata):
MPVYNGTSQAFNFAPLHPDSLNWCPKSSLPPEIPVVDLSKLNSEAELAQFLVDIRKSGLF
YVVNHGVPEELSIGCYNRFRQFCNIPEDQRAEYSTDHHFVNGGYMPYKSSSIGKANKGK
SQKDFVVKYFWRGPRVENRSPDADFKSYHDEYHRRTADIANSVITKILQALTTRFPDFD
PAEYKDNINSHHMFFSNRLYPDNEPEKGENVEYRLVPHRDLSFVTLAHQIPADNGYQGL
FVFTGDGKKVCVPPIRNSYLVFMGQAMSYLTNKYLPAGLHGVDFPEKNCFEGSERSSLI
SFYEPHDHMMPSKALTPKDDEIFDRGCSFFDDIGVDKTGTTYMYVKNKFHEGYYL
PhKabC (Palmaria hecatensis):
MPVYNGTPQAYNGTSQAFNFAPLDADSLTWCPESSLPPEIPAIDLSKVNSKSELAEFLVNI
RKSGLFYVVNHGVPEELSMGCYNHFREFCNIPEEERAEYSTDHHFVHGGYMPYKSSSIG
KANKGKDQKDFVVKYFWRGPLVENRSPSTGFKRYHDEYHRRTSDIANSVIAKIMQALT
TRFPDFDPDEYKKNINSHHMFFSNRLYPDNEPEKGENAEYRLVPHRDLSFVTLAHQIPAD
NGYQGLFVLTGDGKKVCVPPIRNSYLVFMGQAMSYLTNKYLPAGLHGVDFPEKNCFEG
SERSSLISFYEPHDHMMPSKALTPKDDEIFDRGCSFFDDIGVDKSGTAYMHVKNKFHEG
YYL
ReKabC (Rhodophysema elegans):
MAVYNGVSQAFDFAPLDADSLNWCPQSSLPPEIPAIDLGKVNTRAELAQFLIDIRRSGLF
YVVNHGVPEELSIGCYNLFRRFCNIPEEERAKYSTDHHFVNGGYMPYKSSSIGKANMGK
DQKDFVVKYFWRGPRVENRTPTAEFKKYHDEYHRRTSGVADTVIEKIMQALATRFPDF
DPDEYKENTNSHHMFFSNRLYPENEPEKGENVEYRLVPHRDLSLVTLAHQIPADNGYQ
GLFVLTGDGKKVPVPPIRNSYLVFLGQALSYLTNKYLPAGLHGVDFPEKNSFEGSERSSL
ISFYEPHDRMMPSKALTPKEDEVFDRSCSFFDDIGVDTSGTTYMYVKNKFHEGYYL
GfKabC (Grateloupia filicina):
MTSFDFVAKTFDFAPVKADELKWYPRSALPPEIPVIDLNNVNTEEELAQFLVDIRKSGLF
YVVNHDIPEEISIQTYNEFREFCKLPEEARQKYNTDRCFFNGGYVPFKATSINGGNKNKQ
QRDFVVKFFWRGPHVVNRSPNERFAKWHHQYHTRTAELGEKVMTTIAKALKTRFPDF
DPAELEDNVNPHNMIFSNRIYPEFPPSEGEDAEYRLTPHRDISYITLVNQTPANNGFKALF
IVTGDGER VYVPPIRNSYLVFIGQSLSYLTNKYLPAALHGVAFPDGNLEGCERASLVSFY
EPYDRMVPSKNIKPTAEEIAPKSCSFYDSIGCDKTGTTFTDVRAKFHSGYYV
SEQ ID NOs: 6-10, respectively:
>KabC (Digenea simplex)
ATGACAGTAAGTAAGTACAACGGTGTTGCGAGTACGTTCAACGGTGTTGCAAGGAG
ATTCGACTTTGCCCCATTGGAGGCAGATAAATTGAATTGGTATCCCAGGTCAGCTCT
TCCTCCAGAAATCCCTGCTATCGACCTGAATAAGGTCAATACTGAAGAAGAACTGGC
TCAATTCCTGGTCGATATTCGCAAATCTGGGCTCTTTTATATTGTTGATCATGGCATT
CCAGAGGAGATCTCTATAGGATGTTACAATGCATTCCGTGAGTTTTGCAACCTTCCC
GAGGAAGCAAGAGAGAAGTACAACACAGATGAGTCCTTTAAAAGCGGTGGCTATGT
TCCTTTCAAAGGCACGTCGATTGGTGGAGGGAACTTGTTTGAACGACAGAAGGATTT
CGTTGTCAAATTCTTTTGGAGAGGACCAAGTGTTGTCAATAGGTCTCCTAATGATCG
CTTTGCCGAATTCCATGATGAACATCATCGAAAGACAGCTGAGTTGGCCGAAAAAA
TCATCACTACCATTTTGAAAGCCCTGAAGACACGCTTTCCAGAATTCCATCCTGACG
AGCTAAAGGATAATATCAATGTTCGAAACATGTTTTTCAGTAATCGAATCTATCCAG
AGGCTCCGCCAGATGATGGAGAAAAAGCTGACTACCGACTTGTTCCTCATCGAGATC
TCAGTTTTATCACTCTTGCAAATCAAGTTCCGGCTAACAATGGATTCAAGGGTCTTTT
TATACTGACAGGTGATGGAGAAAAAATTCCTGTTCCTCCAATCCGGAACAGCTACTT
GGTCTTCATCGGTCAAGGCCTCTCATATCTCACGAATAAGTATCTTCCTGCAGCGCTT
CATGGTGTGGACTTTCCAGACAATACAAATTTTGAAGGAAGTGAAAGAGCCTCTCTG
ATCAGCTTCTACGAGCCAAACGATTACATGATGCCATCCAAGAACATTAACCCTTTA
CCTGAGGAGATATTTGAGAAAAGCTGTACGTTTTACGATGATGTCGGAGTAGGGAG
GGCTGGTACAACATATAATTACGTGAGGTACAAATTTCATGAAGGATACTACCTTTA
A
>KabC (Rhodophysema elegans)
ATGGCGGTGTACAATGGCGTCTCGCAGGCCTTCGACTTCGCGCCGCTCGACGCCGAC
AGCCTCAACTGGTGCCCCCAGTCGTCCCTGCCGCCCGAGATCCCCGCCATCGACCTC
GGCAAAGTGAACACCAGGGCCGAGCTCGCCCAGTTCCTGATCGACATCCGCAGGTC
CGGCCTCTTCTACGTCGTCAACCACGGCGTGCCGGAAGAGCTCTCGATCGGCTGCTA
CAACCTCTTTCGCCGCTTCTGCAACATCCCCGAGGAGGAGCGCGCCAAGTACAGCAC
CGACCATCATTTCGTCAACGGCGGCTACATGCCATACAAGAGCTCCTCCATCGGCAA
GGCGAACATGGGCAAGGACCAGAAGGACTTTGTCGTCAAGTACTTCTGGAGGGGGC
CCCGCGTGGAGAACAGGACGCCCACCGCCGAGTTTAAGAAGTATCACGATGAGTAC
CATCGCAGGACTTCGGGCGTCGCAGACACGGTCATAGAGAAGATTATGCAGGCGCT
GGCGACGCGCTTTCCGGACTTCGACCCCGACGAGTACAAGGAGAACACGAACAGCC
ATCACATGTTTTTCAGCAACAGGCTCTACCCCGAAAACGAGCCGGAGAAGGGGGAG
AACGTCGAGTACCGCCTTGTTCCGCATCGCGACCTCAGTTTGGTGACGCTCGCGCAC
CAGATCCCCGCAGACAATGGCTACCAGGGCCTGTTCGTTCTTACCGGCGACGGGAA
GAAGGTGCCTGTGCCGCCTATCCGCAACAGCTATCTCGTCTTCCTGGGGCAGGCGTT
GTCTTATCTCACCAACAAGTATCTGCCTGCTGGATTGCATGGCGTGGACTTTCCGGA
GAAGAACTCCTTTGAGGGGAGCGAGCGGTCGTCACTTATTTCATTCTACGAGCCGCA
CGACCGCATGATGCCGTCGAAGGCGCTCACGCCAAAAGAGGATGAAGTTTTTGATA
GAAGCTGCTCCTTTTTCGATGATATTGGTGTTGATACGAGTGGCACGACGTATATGT
ACGTGAAAAATAAGTTCCACGAGGGCTATTATCTATAG
>KabC (Palmaria hecatensis)
ATGGCGGTGTACAATGGCGTCTCGCAGGCCTTCGACTTCGCGCCGCTCGACGCCGAC
AGCCTCAACTGGTGCCCCCAGTCGTCCCTGCCGCCCGAGATCCCCGCCATCGACCTC
GGCAAAGTGAACACCAGGGCCGAGCTCGCCCAGTTCCTGATCGACATCCGCAGGTC
CGGCCTCTTCTACGTCGTCAACCACGGCGTGCCGGAAGAGCTCTCGATCGGCTGCTA
CAACCTCTTTCGCCGCTTCTGCAACATCCCCGAGGAGGAGCGCGCCAAGTACAGCAC
CGACCATCATTTCGTCAACGGCGGCTACATGCCATACAAGAGCTCCTCCATCGGCAA
GGCGAACATGGGCAAGGACCAGAAGGACTTTGTCGTCAAGTACTTCTGGAGGGGGC
CCCGCGTGGAGAACAGGACGCCCACCGCCGAGTTTAAGAAGTATCACGATGAGTAC
CATCGCAGGACTTCGGGCGTCGCAGACACGGTCATAGAGAAGATTATGCAGGCGCT
GGCGACGCGCTTTCCGGACTTCGACCCCGACGAGTACAAGGAGAACACGAACAGCC
ATCACATGTTTTTCAGCAACAGGCTCTACCCCGAAAACGAGCCGGAGAAGGGGGAG
AACGTCGAGTACCGCCTTGTTCCGCATCGCGACCTCAGTTTGGTGACGCTCGCGCAC
CAGATCCCCGCAGACAATGGCTACCAGGGCCTGTTCGTTCTTACCGGCGACGGGAA
GAAGGTGCCTGTGCCGCCTATCCGCAACAGCTATCTCGTCTTCCTGGGGCAGGCGTT
GTCTTATCTCACCAACAAGTATCTGCCTGCTGGATTGCATGGCGTGGACTTTCCGGA
GAAGAACTCCTTTGAGGGGAGCGAGCGGTCGTCACTTATTTCATTCTACGAGCCGCA
CGACCGCATGATGCCGTCGAAGGCGCTCACGCCAAAAGAGGATGAAGTTTTTGATA
GAAGCTGCTCCTTTTTCGATGATATTGGTGTTGATACGAGTGGCACGACGTATATGT
ACGTGAAAAATAAGTTCCACGAGGGCTATTATCTATAG
>KabC (Palmaria palmata)
ATGCCCGTGTACAACGGCACCTCCCAGGCCTTCAACTTCGCGCCCCTCCACCCCGAC
AGCCTCAACTGGTGCCCCAAGTCGTCCCTGCCCCCCGAGATCCCCGTAGTCGACCTT
AGCAAACTCAACTCTGAGGCCGAGCTCGCCCAGTTTCTGGTCGACATCCGCAAGTCC
GGCCTCTTCTACGTTGTCAACCACGGCGTGCCGGAGGAGCTCTCCATCGGCTGCTAC
AACCGCTTCCGCCAGTTTTGCAACATCCCCGAGGACCAGCGCGCCGAGTACAGCAC
CGACCACCATTTTGTAAACGGCGGCTACATGCCCTACAAAAGCAGTTCCATTGGCAA
GGCGAACAAGGGCAAGAGTCAGAAGGACTTTGTCGTCAAGTACTTCTGGAGGGGGC
CCCGTGTTGAGAACAGGTCGCCCGACGCCGACTTCAAGAGCTATCACGACGAGTAC
CACCGGAGGACCGCCGACATCGCGAACAGCGTCATCACCAAAATCCTGCAGGCGTT
GACAACCCGCTTCCCAGACTTTGACCCCGCTGAGTACAAGGACAACATAAACAGCC
ACCACATGTTTTTCAGCAATCGCCTGTATCCCGACAACGAGCCGGAGAAAGGAGAG
AACGTGGAGTACCGCCTGGTTCCGCATCGAGATCTCAGTTTCGTCACCCTCGCCCAC
CAGATCCCCGCAGACAACGGCTACCAGGGCCTGTTCGTTTTCACCGGCGACGGGAA
GAAGGTCTGTGTGCCGCCCATCCGCAACAGCTACCTCGTCTTCATGGGGCAGGCGAT
GTCCTATCTCACCAACAAGTACCTGCCTGCCGGCCTGCATGGTGTCGACTTTCCGGA
GAAGAACTGTTTTGAGGGCAGCGAGCGGTCGTCCCTTATTTCGTTTTACGAGCCGCA
CGACCACATGATGCCTTCGAAGGCGCTGACCCCGAAAGATGATGAAATTTTTGATAG
AGGCTGCTCCTTTTTCGATGATATTGGTGTCGATAAGACTGGCACGACTTATATGTA
CGTCAAAAATAAGTTCCACGAGGGATATTATCTATAG
>KabC (Grateloupia filicina)
ATGACTTCGTTTGATTTCGTTGCGAAGACCTTTGACTTTGCTCCCGTGAAAGCAGATG
AACTGAAATGGTACCCCAGGTCTGCTCTCCCTCCAGAAATCCCTGTCATCGATCTGA
ACAACGTCAACACTGAAGAAGAGCTGGCTCAATTCTTGGTTGATATTCGCAAATCTG
GACTCTTCTATGTGGTCAACCATGACATTCCAGAAGAGATATCCATCCAAACTTACA
ACGAATTTCGTGAGTTTTGCAAGCTCCCTGAGGAGGCCAGACAGAAGTATAACACT
GATAGATGCTTCTTTAATGGCGGTTACGTTCCTTTCAAAGCCACTTCAATTAATGGA
GGCAACAAGAACAAACAGCAGAGAGATTTTGTCGTAAAATTCTTCTGGAGAGGGCC
GCATGTCGTCAACAGGAGTCCGAACGAGCGCTTCGCAAAATGGCACCACCAATATC
ACACAAGAACTGCTGAACTAGGTGAAAAGGTCATGACTACCATCGCGAAAGCTTTG
AAGACACGCTTTCCGGATTTTGACCCTGCTGAGTTGGAAGACAACGTCAACCCGCAC
AACATGATTTTCAGTAATCGAATCTACCCAGAGTTTCCACCCAGCGAAGGAGAGGAT
GCCGAATACCGACTTACTCCTCATAGGGATATCAGTTATATCACCCTCGTGAATCAA
ACTCCGGCAAACAATGGCTTTAAGGCTCTTTTCATAGTAACTGGTGATGGGGAGAGA
GTCTATGTTCCTCCCATCCGCAACAGTTACTTGGTATTCATTGGACAGAGCCTTTCAT
ATCTCACCAACAAGTATCTTCCTGCCGCCCTGCATGGGGTGGCCTTTCCAGACGGAA
ATTTAGAAGGATGTGAGAGGGCATCTTTGGTTTCCTTCTACGAGCCTTACGATCGTA
TGGTGCCTTCAAAAAACATCAAGCCCACAGCAGAGGAGATTGCTCCGAAAAGCTGT
TCATTTTACGATTCTATCGGATGTGATAAGACTGGCACGACATTTACGGACGTGAGG
GCGAAATTTCATTCCGGATACTATGTTTGA
SEQ ID NOs: 11-15, respectively:
DsKabA (Digenea simplex):
MSITTMKGVTSESPAEALSRFQTTGLTLNNPKDLYWMTEFLKEEFYDKGNYYYPIKTVC
DGELIETELFCPFEPKLSPHYIQLYNSRDERSNLYAVPPKKTDMKKYNRINCEKMGSLM
APNSNYDDTEMVSLFYSMMYYLNDQTAHLKLPEEDIQPELVDELNDHVLQYLSVFLSIF
KPREPQDLERIWNFLDFYQPYFKKVDGKIILHEKYQGRTPPQIGLIKKITGYVLERFAPKK
NITQVIYEVIRYIKGIKQEIKIRGDKSFTLSLKEYDEFRDQVTSSPMAHSITDLTYDDFSYK
AYMNPLFIKLEDLTSEIITYFNDVCTCDRERLDDDPFNSVFILRDLHSLNYVKSCDLVVK
HAHDKLSKFLEIKQTLLKESTNENEKKAIAQMIKTREDSLIGYTIHEICCVINGYARDHK
PLMKDYLEKNIFKKLKATN
PpKabA (Palmaria palmata):
MLPPAFLPPAVHSLFRGGLLQRPEHVGPTPTATPSAASIRAHRAPTTLVMVAHGRRRLER
PTAALPTGARRTGLTLTDPADLHSMVAHLKKENFDKGNCNYPIKTVCDGELIDVQFAFP
FEPKLSPHYAALYNSRDERSQLYAVPPSTTDMKKYNRINCEKMGALLAPNSAYGDTEIV
SLFYSMMYYLNDQTAHFKLPEEEIQYELVDELNDNVLQYLSIFLGVFKPRDAADLERIW
DFLEFYQPYFHKVDGRIELDAKYSGQTPPQVALVSKIAGYAADRFGASKNITQIIYEVIR
YVKGIKEEIKIRCDKGFSLTLSEYDAFRDNVTSSPMAHSVTDLTHDAFSYEAYTNPVFNE
LENLTSQVITYVNDVCTCDRERLDDDPFNSVFILKNRDGLNFAEACDRVVGEVVKKTAR
FLETKERLLAEAADEDGRAAMAQMIKTREDSIIGYMLHEVCCVTDGYARDHKPLMKDY
LEKTMFGEVASGAM
PhKabA (Palmaria hecatensis):
MPPPAFLPPAVRSLFRGAPLQRLQHVGPIQSATPSAASIRARRAARTLVMVDAGPERPTA
APPTGARRTDLTLTDPADLALMVAHLKREHFDKGYCTYPIKTVCDGELIDVQFSFPFEPK
LSPHYAALYNSRDERSQLYAVSPNTTDMKKYNRINCEKMGALLAPNSAYGDTEMVSLF
YSMMYYLNDQTAHFKLPEEEIQYELVDELNDNVLQYLSIFLGVFEPRDAADLERIWDFL
EFYQPYFNKVGGKIVLDAKYSGQTPPQVALVTKIAGYAAGREGATKNITQIIYEVIRYVK
GIKQEIKIRCDKSFTLNLAEYDAFRDQVTSSPMAHSVTDLTHDAFSYKAYTNPVENDLE
NLTSQIITYVNDVCTCDRERLDDDPFNSVFILKNRDGLNFADACDLVVGEVVKKTAKFL
ETKDRLLAEAADEEGRAAIAQKIKTREDSIIGYMMHEVCCVTDGYARDHKPLMKEYLE
KAMFGEASSGAL
ReKabA (Rhodophysema elegans):
MSPSAFLPLAGPLAFRRASLQRLNHTRTLPPTVAPSRAPMTPFMVDTSRGHPATAAPPM
GARRSLTLTDPADLALMVSHLKREHFDKGDYLYPIKTVCAGEDIDVRFAIPFEPRLSSHY
AALYNSRNDRAKLYAVPPRTTDMRKYNRINCEKMGALLAPNSAYGDTEAVSLFYSMM
YYLNDQTAICKLPEDEIELRLVDELNDNVLQYLGIFLSVFEPRDAADLERIWDFLDFYQP
YFRKAGRRIVLDDKYLGQTPPQVALVTTIANYVAERFGATKNITQVVYEVIRYVKGIKQ
EVKIRCDKGFTLSLAEYDDFRDQVTSSPMAHSVTDLTHDAFSYEAYKNPVFNSLENLTS
QVITYINDVCTCDRERLDKDPFNSVFILKDRDGLNFADACDLVVAEIEKKMAKFLETKK
QLLSEAADEERRTAMAQMIKTREDSIIGYMMHEVCCVTDGYARDHKPKMKEYLEKAM
FGEVSREAM
GfKabA partial sequence (Grateloupia filicina):
MNLILRSNMPIFPSCAFLSSSTTTTFIQPLVKRIHSVCPNPVRHEPVSANRRLVMRLAWNP
TPTQQLTTESPTEALSRLQTTGLTLTDPKDLYWMTDFLEQQFYSKRNPNYPMKTICDGE
LIETEFHCPYQPKLSPHYMRLCNTKHERSLLYSIPPNTTDMNKYNRINCEKFASLVAPNS
NYEDTEAVALMYSMMYYLNDQTAHLKLPEDMIQPQLIDELNDNVLQYLAVFLSIFEPR
DPEDLERIWDFLDFYQKYFNKIDGKIVLDEKYKGPVPPQIALINKITQYISKRFAPTKNITQ
FIYEVIRYIKGIKQEVYIRCDKSFTLSLKEYDEFRDQVTSSPMAHAVTDMTYDNFSYKLY
LNPLFTELENLTSELITYFNDVCTCDRERLDNDPFNSVFILKDLYGGTYAQSCDLVVSET
RKKFSKFLEIKQILLDGAADETEKQAIAQMIK
SEQ ID NOs: 16-20, respectively:
DsKabA (Digenea simplex):
ATGAGTATTACCACTATGAAAGGAGTTACATCAGAGAGTCCTGCCGAAGCGCTTTCC
CGCTTCCAAACTACTGGATTGACGTTGAACAACCCCAAGGACCTTTACTGGATGACA
GAATTTTTGAAGGAAGAATTTTACGACAAGGGAAACTATTATTATCCCATCAAAACG
GTATGTGATGGCGAACTCATCGAAACCGAGCTCTTCTGTCCCTTCGAACCAAAGCTC
AGTCCACATTACATTCAATTATACAACTCTCGTGATGAGCGATCCAACTTGTACGCC
GTTCCGCCCAAGAAAACAGACATGAAAAAGTACAATCGTATAAATTGTGAGAAAAT
GGGTTCTTTGATGGCCCCTAATTCCAATTATGACGACACAGAAATGGTTTCTCTCTTC
TACTCCATGATGTACTACCTCAATGATCAGACCGCTCATCTTAAGCTCCCTGAAGAA
GATATTCAACCTGAGCTTGTTGATGAACTCAACGACCATGTCTTACAGTATCTAAGT
GTGTTCCTTAGCATCTTTAAACCGCGTGAACCTCAAGATTTGGAGCGAATCTGGAAT
TTTTTGGACTTTTACCAGCCATATTTCAAGAAAGTTGATGGCAAGATTATCTTGCACG
AGAAATATCAGGGGCGTACTCCACCACAAATCGGTTTGATCAAGAAAATAACAGGT
TATGTATTAGAGAGGTTTGCTCCGAAAAAGAATATCACTCAAGTCATCTACGAGGTC
ATCAGATATATCAAGGGAATCAAGCAAGAAATTAAGATTCGAGGTGATAAGAGCTT
CACTCTGTCTCTCAAAGAGTATGATGAGTTCCGTGACCAAGTTACGTCCAGCCCAAT
GGCTCATTCGATTACAGATCTTACTTATGACGACTTCTCTTACAAGGCATACATGAA
CCCACTCTTCATCAAGCTGGAAGATCTAACCTCTGAGATCATCACGTACTTCAACGA
CGTTTGCACATGTGATCGCGAGAGACTGGACGATGATCCTTTCAACTCTGTTTTCATT
CTGAGAGATCTTCACAGTCTCAACTATGTGAAGTCATGTGATCTTGTCGTTAAGCAT
GCACATGACAAGCTATCAAAATTCTTGGAGATCAAGCAGACTCTTTTGAAAGAATCT
ACAAACGAAAATGAGAAAAAGGCCATTGCCCAGATGATTAAGACTCGTGAAGATTC
CTTGATTGGATATACGATTCATGAAATTTGCTGTGTTACAAATGGGTATGCTCGTGA
CCACAAGCCCTTGATGAAAGATTATTTGGAGAAAAATATCTTTAAGAAACTCAAGG
CGACAAATTAA
PpKabA (Palmaria palmata):
ATGCTTCCTCCTGCCTTCCTCCCTCCGGCCGTGCACTCCCTCTTCCGCGGCGGCCTGC
TGCAGCGTCCGGAACACGTTGGTCCAACCCCAACTGCAACGCCTTCCGCCGCTTCGA
TCCGCGCACACCGCGCACCAACGACGCTCGTCATGGTCGCGCACGGTCGGCGCCGT
CTCGAACGCCCCACAGCGGCACTACCAACCGGCGCGCGACGGACCGGCTTGACGCT
CACCGACCCGGCCGACCTGCATTCCATGGTGGCGCACCTGAAAAAGGAGAACTTCG
ACAAGGGCAACTGCAACTACCCAATCAAGACGGTGTGCGATGGAGAACTCATCGAC
GTCCAGTTCGCCTTCCCGTTTGAACCGAAGCTGAGCCCGCACTACGCCGCCCTGTAC
AATTCGCGTGACGAGCGCTCCCAGCTGTACGCTGTCCCGCCTAGTACTACAGATATG
AAAAAATACAACCGCATCAACTGCGAGAAGATGGGCGCCCTGCTCGCGCCGAACTC
TGCGTATGGCGACACGGAGATAGTGTCGCTGTTCTATTCGATGATGTATTATCTCAA
TGACCAGACGGCGCACTTCAAGCTACCAGAGGAAGAGATCCAGTACGAGCTCGTGG
ACGAGCTCAACGACAATGTGCTGCAGTATCTCAGCATATTTCTGGGCGTTTTCAAGC
CGCGCGATGCTGCGGATTTGGAGCGCATTTGGGACTTTCTCGAGTTTTACCAGCCCT
ATTTTCACAAGGTCGACGGGAGGATTGAGTTAGATGCCAAGTATAGCGGGCAGACG
CCGCCGCAGGTCGCGCTTGTCTCGAAGATTGCGGGGTACGCCGCGGATCGGTTCGGC
GCGTCAAAGAACATCACCCAGATCATCTACGAGGTCATTCGATATGTCAAGGGCATC
AAGGAGGAGATCAAGATCAGGTGCGACAAGGGCTTTAGCCTCACCCTCTCCGAGTA
CGACGCGTTCCGGGACAATGTCACATCGAGCCCGATGGCGCACTCCGTAACCGACC
TGACGCACGATGCGTTCTCCTACGAGGCCTACACGAACCCCGTCTTCAACGAGCTGG
AAAACCTGACCTCCCAAGTCATCACCTACGTCAACGACGTGTGTACGTGCGACCGG
GAGCGGCTGGACGACGACCCGTTCAATTCGGTCTTTATTCTGAAAAATCGCGACGGT
CTGAACTTCGCCGAAGCGTGCGATCGCGTTGTCGGCGAGGTGGTGAAGAAGACGGC
AAGGTTCCTGGAGACGAAGGAGCGGCTTCTTGCGGAGGCTGCTGATGAAGACGGGA
GGGCGGCCATGGCGCAGATGATCAAGACGCGCGAAGACTCGATCATCGGGTACATG
CTGCACGAGGTCTGCTGCGTGACGGATGGGTATGCGCGCGACCACAAGCCGTTGAT
GAAAGACTATCTGGAGAAGACGATGTTTGGTGAGGTGGCCAGCGGAGCAATGTAG
PhKabA (Palmaria hecatensis):
ATGCCTCCTCCCGCCTTCCTCCCTCCAGCCGTGCGCTCCCTCTTCCGCGGCGCCCCGC
TGCAGCGTCTGCAACACGTCGGTCCAATCCAAAGTGCAACGCCCTCCGCTGCTTCGA
TCCGCGCACGCCGTGCAGCAAGGACGCTCGTCATGGTCGACGCAGGCCCGGAGCGC
CCCACAGCAGCACCACCAACCGGCGCGCGACGGACCGACTTGACGCTAACGGACCC
GGCAGATCTGGCGCTCATGGTCGCCCATCTGAAAAGGGAGCACTTCGACAAGGGGT
ACTGCACCTACCCGATAAAGACGGTGTGCGATGGAGAACTCATCGACGTCCAGTTCT
CCTTCCCGTTCGAGCCGAAGCTGAGCCCGCACTACGCGGCCCTGTACAATTCGCGTG
ACGAGCGCTCGCAGCTGTACGCTGTCTCGCCAAATACTACGGATATGAAGAAGTAC
AACCGCATCAACTGCGAGAAGATGGGCGCGCTGCTTGCCCCGAACTCTGCATATGG
CGACACGGAGATGGTTTCGCTGTTCTACTCGATGATGTATTATCTCAATGACCAGAC
GGCGCACTTCAAGCTGCCCGAGGAGGAGATCCAGTACGAGCTTGTGGACGAGCTCA
ACGACAATGTGCTGCAGTATCTCAGCATTTTTCTGGGCGTGTTCGAGCCGCGCGATG
CTGCGGACTTGGAGCGTATTTGGGACTTTCTCGAGTTTTACCAGCCGTATTTTAACAA
GGTCGGCGGGAAGATCGTGCTGGATGCCAAGTACAGCGGGCAGACGCCGCCGCAGG
TCGCGCTCGTCACGAAGATCGCGGGGTACGCCGCGGGGCGCTTCGGCGCGACGAAG
AACATCACGCAGATCATCTACGAGGTCATTCGATATGTTAAGGGCATCAAGCAGGA
GATCAAGATCAGGTGCGACAAGAGCTTTACCCTCAACCTCGCGGAGTACGACGCGT
TCCGCGACCAGGTCACATCGAGCCCGATGGCGCACTCTGTAACCGACTTGACGCAC
GACGCCTTCTCCTACAAGGCCTACACGAACCCCGTCTTCAACGACCTGGAAAACCTG
ACCTCCCAAATCATCACCTATGTCAACGACGTATGCACTTGTGACCGCGAGCGGCTG
GACGACGATCCGTTCAACTCGGTCTTTATTCTGAAAAATCGCGACGGCCTCAACTTC
GCCGACGCGTGCGACCTCGTTGTCGGCGAGGTCGTAAAGAAGACGGCGAAGTTCCT
GGAGACTAAGGATAGGCTTCTTGCGGAAGCTGCTGATGAAGAGGGGAGGGCGGCTA
TTGCGCAGAAGATCAAGACGCGCGAGGACTCGATCATCGGGTACATGATGCACGAG
GTTTGCTGCGTGACGGACGGGTATGCGCGCGACCACAAGCCGCTGATGAAAGAGTA
TCTGGAGAAGGCGATGTTTGGTGAGGCGAGCAGCGGAGCCCTGTAG
ReKabA (Rhodophysema elegans):
ATGTCTCCTTCGGCCTTTCTCCCCCTAGCCGGGCCCTTAGCCTTCCGGCGGGCCTCGC
TGCAGCGTCTAAATCATACCCGTACCCTCCCACCCACCGTCGCTCCGTCCCGCGCAC
CAATGACGCCCTTCATGGTCGACACCAGCCGCGGGCACCCTGCTACAGCAGCCCCA
CCTATGGGCGCGCGCCGCAGCCTGACGCTGACGGACCCGGCCGATCTGGCGCTCAT
GGTCTCCCACCTGAAGCGCGAGCATTTCGACAAGGGCGACTACCTTTACCCGATCAA
AACCGTATGCGCCGGCGAGGACATCGACGTCCGCTTCGCTATCCCCTTCGAGCCGCG
CCTCAGCTCGCACTATGCGGCCCTGTACAACTCGCGAAACGATCGCGCCAAGCTTTA
CGCCGTCCCGCCGCGGACGACTGACATGCGTAAGTACAACCGCATCAACTGTGAGA
AGATGGGCGCCCTGCTCGCGCCCAACTCTGCCTACGGCGACACAGAGGCCGTCTCTC
TCTTCTACTCGATGATGTACTACCTCAACGACCAGACGGCGATCTGCAAGCTGCCCG
AGGACGAAATCGAGCTTCGCCTCGTCGACGAGCTCAACGACAACGTGCTCCAATAT
CTCGGCATCTTCCTCTCCGTCTTCGAGCCGCGCGATGCCGCTGATTTGGAGCGTATCT
GGGACTTTCTAGACTTTTACCAGCCGTACTTCCGCAAGGCCGGCCGGAGGATCGTGC
TGGACGACAAATACCTCGGGCAGACGCCGCCGCAGGTCGCGCTTGTCACAACGATC
GCAAACTACGTCGCCGAACGCTTCGGCGCGACGAAGAACATCACGCAGGTCGTCTA
CGAGGTGATTCGCTATGTTAAGGGCATCAAGCAGGAGGTGAAGATCAGGTGCGACA
AGGGATTCACCCTCTCACTCGCCGAGTACGACGATTTCCGCGACCAGGTCACGTCCA
GCCCGATGGCGCACTCCGTGACCGACCTCACCCACGACGCCTTCTCCTACGAGGCAT
ATAAGAACCCGGTCTTCAACTCCCTGGAGAACCTGACCTCCCAGGTCATTACCTACA
TCAACGACGTGTGCACGTGTGACCGAGAGCGGCTAGACAAGGACCCGTTTAACTCG
GTGTTCATTCTGAAAGACCGCGACGGTCTGAACTTCGCCGACGCGTGCGACCTCGTC
GTCGCCGAGATCGAAAAGAAAATGGCAAAGTTCCTCGAGACGAAGAAGCAGCTTCT
GTCGGAGGCTGCTGATGAAGAGCGCAGAACAGCCATGGCGCAGATGATCAAGACGC
GGGAGGACTCGATCATCGGCTACATGATGCACGAGGTCTGCTGCGTGACGGACGGG
TATGCGCGCGACCACAAGCCGAAGATGAAGGAGTATCTGGAGAAGGCGATGTTTGG
GGAGGTGAGCAGGGAAGCCATGTAG
GfKabA partial sequence (Grateloupia filicina):
ATGAATTTGATCCTCCGCAGTAACATGCCTATTTTCCCATCTTGCGCTTTTCTCTCAT
CCTCAACCACTACCACTTTCATTCAACCTCTTGTCAAACGTATCCACTCCGTTTGCCC
CAATCCAGTACGCCATGAGCCAGTTTCAGCAAACCGTCGCCTAGTCATGCGACTTGC
CTGGAATCCTACACCCACTCAACAACTTACCACAGAGAGTCCTACCGAAGCACTTTC
CCGCCTTCAAACCACTGGACTAACCCTGACTGACCCGAAGGACCTTTACTGGATGAC
CGATTTTTTGGAACAACAATTTTACTCAAAAAGAAACCCCAACTATCCCATGAAAAC
CATCTGCGACGGCGAACTCATCGAGACCGAATTTCACTGTCCATACCAACCCAAACT
CAGCCCTCATTATATGAGGTTGTGCAACACTAAGCATGAAAGATCTCTCTTGTACTC
AATTCCACCAAACACCACAGATATGAACAAGTACAACCGAATTAATTGCGAGAAGT
TCGCCTCTTTGGTAGCCCCGAATTCCAATTATGAAGATACAGAGGCTGTTGCCCTCA
TGTACTCCATGATGTACTATCTCAATGATCAGACTGCCCATCTTAAACTTCCTGAGG
ATATGATTCAGCCTCAACTTATTGATGAACTCAACGATAACGTATTGCAGTATCTCG
CTGTGTTTCTAAGCATCTTCGAACCCCGCGATCCGGAAGATTTGGAGCGCATCTGGG
ACTTCTTAGACTTTTATCAGAAGTACTTCAACAAAATAGACGGAAAGATTGTCTTGG
ATGAAAAGTACAAAGGACCAGTCCCGCCTCAAATTGCTTTGATTAACAAAATCACCC
AATATATCTCGAAGAGATTTGCTCCGACGAAGAACATAACTCAGTTCATCTACGAGG
TCATCAGATACATCAAGGGGATCAAGCAAGAGGTCTACATTCGATGTGATAAGAGT
TTTACTCTTTCTCTCAAGGAGTATGATGAATTTCGTGATCAAGTGACGTCCAGCCCG
ATGGCACATGCTGTGACAGATATGACCTATGACAACTTCTCTTATAAGCTCTACCTG
AACCCGCTTTTCACCGAACTGGAGAATCTGACTTCAGAACTGATCACCTACTTCAAC
GACGTTTGCACATGTGATCGCGAGAGACTGGACAATGATCCTTTCAACTCTGTGTTC
ATTCTGAAAGACCTTTATGGTGGTACCTATGCACAAAGTTGTGATCTTGTCGTGAGC
GAAACGCGCAAAAAGTTCTCTAAATTTTTGGAGATCAAACAAATTCTTCTTGATGGG
GCAGCAGACGAAACCGAGAAACAAGCTATTGCCCAGATGATTAAG

### EXEMPLARY EMBODIMENTS

1. A process for preparing a compound having the formula,

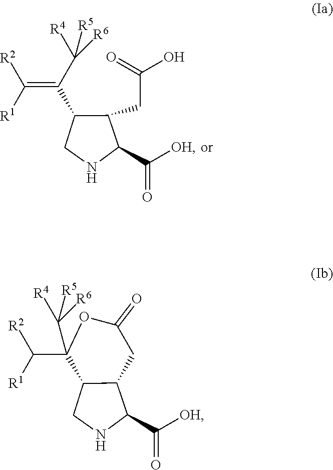

or a salt thereof, wherein the compound or a salt of Formula (Ia) or (Ib) is prepared by cyclizing a compound of formula II:

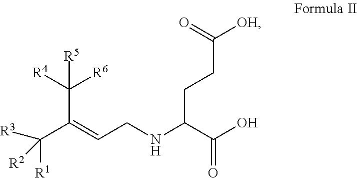

or a salt thereof; wherein the cyclization comprises contacting compound of Formula II with a recombinant KabC polypeptide; wherein
(a) at least one of R¹, R², R³, R⁴, R⁵ or R⁶ is hydrogen; and
(b) R¹ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R² is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R³ is hydrogen, halogen, —CN, —C(O)OH, —CONH₂, —NO₂, —SO₂Cl, —SO₂NH₂, —NHNH₂, —NHSO₂CH₃, —N₃, —NR^{3A}R^{3B}, —OR^{3A}, —SR^{3A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁴ is hydrogen, halogen, —CN, —C(O)OH, —CONH₂, —NO₂, —SO₂Cl, —SO₂NH₂, —NHNH₂, —NHSO₂CH₃, —N₃, —NR^{4A}R^{4B}, —OR^{4A}, —SR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁵ is hydrogen, halogen, —CN, —C(O)OH, —CONH₂, —NO₂, —SO₂Cl, —SO₂NH₂, —NHNH₂, —NHSO₂CH₃, —N₃, —NR^{5A}R^{5B}, —OR^{5A}, —SR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁶ is hydrogen, halogen, —CN, —C(O)OH, —CONH₂, —NO₂, —SO₂Cl, —SO₂NH₂, —NHNH₂, —NHSO₂CH₃, —N₃, —NR^{6A}R^{6B}, —OR^{6A}, —SR^{6A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; any two of R¹, R² or R³ substituents or any two of R⁴, R⁵ or R⁶ substituents may optionally be joined to form a substituted or unsubstituted aryl or substituted or unsubstituted heteroaryl;
   any one of R¹, R² or R³ and any one of R⁴, R⁵ or R⁶ may optionally be joined to form a substituted or unsubstituted cycloalkyl, substituted or unsubstituted aryl or substituted or unsubstituted heteroaryl; and
   each R^{1A}, R^{2A}, R^{3A}, R^{4A}, R^{5A}, R^{6A}, RIB, R^{2B}, R^{3B}, R^{4B}, R^{5B}, and R^{6B} is independently a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.
2. The process of embodiment 1, wherein
(a) at least one of R¹, R², R³, R⁴, R⁵ or R⁶ is hydrogen; and
(b) R¹ is hydrogen, substituted or unsubstituted C₁-C₁₀ alkyl, substituted or unsubstituted C₅-C₁₀ aryl, or substituted or unsubstituted 5 to 10 membered heteroaryl;
   R² is hydrogen, substituted or unsubstituted C₁-C₁₀ alkyl, substituted or unsubstituted C₅-C₁₀ aryl, or substituted or unsubstituted 5 to 10 membered heteroaryl;
   R₃ is hydrogen, substituted or unsubstituted C₁-C₁₀ alkyl, substituted or unsubstituted C₅-C₁₀ aryl, or substituted or unsubstituted 5 to 10 membered heteroaryl;
   R⁴ is hydrogen, substituted or unsubstituted C₁-C₁₀ alkyl, substituted or unsubstituted C₅-C₁₀ aryl, or substituted or unsubstituted 5 to 10 membered heteroaryl;
   R⁵ is hydrogen, substituted or unsubstituted C₁-C₁₀ alkyl, substituted or unsubstituted C₅-C₁₀ aryl, or substituted or unsubstituted 5 to 10 membered heteroaryl;
   R⁶ is hydrogen, substituted or unsubstituted C₁-C₁₀ alkyl, substituted or unsubstituted C₅-C₁₀ aryl, or substituted or unsubstituted 5 to 10 membered heteroaryl;
   any two of R¹, R² or R³ substituents or any two of R⁴, R⁵ or R⁶ substituents may optionally be joined to form a substituted or unsubstituted C₅-C₁₀ aryl or substituted or unsubstituted 5 to 10 membered heteroaryl; and
   any one of R¹, R² or R³ and any one of R⁴, R⁵ or R⁶ may optionally be joined to form a substituted or unsubstituted C₃-C₈ cycloalkyl, substituted or unsubstituted C₅-C₁₀ aryl or substituted or unsubstituted 5 to 10 membered heteroaryl.
3. The process of embodiment 1, wherein
(a) at least one of R¹, R², R³, R⁴, R⁵ or R⁶ is hydrogen; and
(b) R¹ is hydrogen or substituted or unsubstituted C₁-C₁₀ alkyl; R² is hydrogen or substituted or unsubstituted C₁-C₁₀ alkyl; R³ is hydrogen or substituted or unsubstituted C₁-C₁₀ alkyl; R⁴ is hydrogen or substituted or unsubstituted C₁-C₁₀ alkyl; R⁵ is hydrogen or substituted or unsubstituted C₁-C₁₀ alkyl; R⁶ is hydrogen or substituted or unsubstituted C₁-C₁₀ alkyl;
   any two of R¹, R² or R³ substituents or any two of R⁴, R⁵ or R⁶ substituents may optionally be joined to form a substituted or unsubstituted C₅-C₁₀ aryl or substituted or unsubstituted 5 to 10 membered heteroaryl; and
   any one of R¹, R² or R³ and any one of R⁴, R⁵ or R⁶ may optionally be joined to form a substituted or unsubstituted C₃-C₈ cycloalkyl, substituted or unsubstituted C₅-C₁₀ aryl or substituted or unsubstituted 5 to 10 membered heteroaryl.
4. The process of any one of embodiments 1 to 3, wherein R¹, R², R³, R⁴, R⁵ and R⁶ are hydrogen.
5. The process of any one of embodiments 1 to 4, wherein the process comprises contacting the compound of Formula II, or a salt thereof, with a purified KabC polypeptide and a α-keto-glutarate.
6. The process of any one of embodiments 1 to 5, wherein the process for preparing the compound of Formula II, or a salt thereof, comprises:
   a. contacting a compound of Formula III:

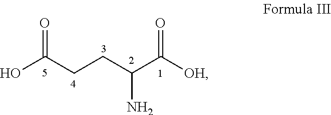

or a salt thereof, with a compound of Formula IV:

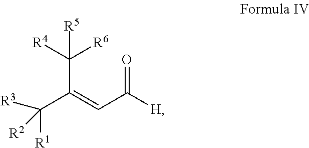

or a salt thereof, to form an imine, and
   b. reducing the imine to produce the compound of Formula II.
7. The process of any one of embodiments 1 to 6, wherein the contacting comprises contacting the compound of Formula II, or a salt thereof, with a recombinant microorganism expressing the recombinant KabC polypeptide.
8. The process of any one of embodiments 1-7, wherein the recombinant KabC polypeptide comprises an amino acid sequence that is at least 80% identical to any of SEQ ID NOs: 1-5.
9. The process of any one of embodiments 1-7, wherein the recombinant KabC polypeptide is encoded by a polynucleotide comprising a sequence that is at least 80% identical to any of SEQ ID NOs: 6-10.
10. The process of any one of embodiments 7-9, wherein the microorganism further expresses a recombinant KabA polypeptide.
11. The process of embodiment 10, wherein the recombinant KabA polypeptide comprises an amino acid sequence that is at least 80% identical to any of SEQ ID NOs: 11-15.
12. The process of embodiment 10, wherein the recombinant KabA polypeptide is encoded by a polynucleotide comprising a sequence that is at least 80% identical to any of SEQ ID NOs: 16-20.
13. The process of any one of embodiments 7-12, wherein the recombinant microorganism is a bacterium.
14. The process of embodiment 13, wherein the bacterium is E. coli.
15. A process for preparing a compound having the formula,

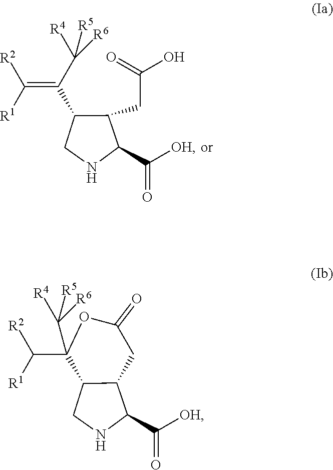

or a salt thereof, wherein the process comprises:
contacting a compound of Formula III:

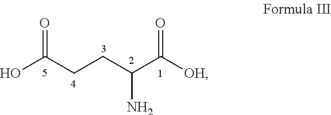

or a salt thereof, with a compound of Formula V:

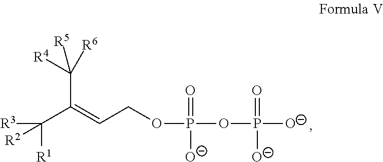

or a salt thereof, a recombinant KabA polypeptide, a recombinant KabC polypeptide, and a α-keto-glutarate.
16. The process of embodiment 15, wherein the recombinant KabA polypeptide and recombinant KabC polypeptide are each expressed by a recombinant microorganism.
17. The process of embodiment 15 or 16, wherein the recombinant KabC polypeptide comprises an amino acid sequence that is at least 80% identical to SEQ ID NO: 1.
18. The process of any one of embodiments 15-17, wherein the recombinant KabC polypeptide is encoded by a polynucleotide that is at least 80% identical to SEQ ID NO: 2.
19. The process of any one of embodiments 15-18, wherein the recombinant KabA polypeptide comprises an amino acid sequence that is at least 80% identical to SEQ ID NO: 3.
20. The process of any one of embodiments 15-19, wherein the recombinant KabA polypeptide is encoded by a polynucleotide that is at least 80% identical to SEQ ID NO: 4.
21. The process of any one of embodiments 15-20, wherein the recombinant KabA polypeptide and the recombinant KabC polypeptide are expressed by the same recombinant microorganism.
22. The process of any one of embodiments 15-21, wherein the microorganism is a bacterium.
23. The process of embodiment 22, wherein the bacterium is E. coli.
24. A compound of Formula II:

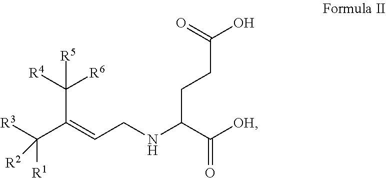

or a salt thereof.
25. A compound of Formula VI:

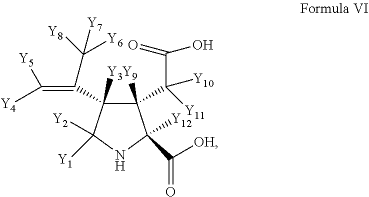

or a salt thereof, wherein:
one of Y₁, Y₂, Y₃, Y₄, Y₅, Y₆, Y₇, Y₈, Y₉, Y₁₀, Y₁₁, or Y₁₂ is a hydrogen that is isotopically enriched with deuterium or tritium, and the rest of Y₁, Y₂, Y₃, Y₄, Y₅, Y₆, Y₇, Y₈, Y₉, Y₁₀, Y₁₁, and Y₁₂ are non-enriched hydrogen atoms.
26. A compound of Formula VII:

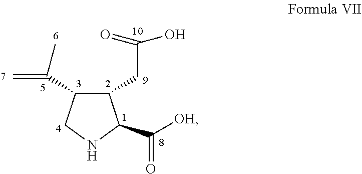

or a salt thereof, wherein:
1, 2, 3, 4, 5, 6, 7, 8, 9, and 10 are carbon atoms;
and at least one of 1, 2, 3, 4, 5, 6, 7, 8, 9, or 10 carbon atom is isotopically enriched with carbon-13 or carbon-14.
27. A compound of Formula VIII:

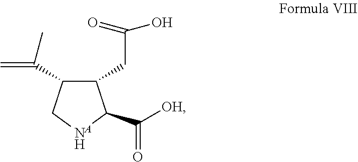

or a salt thereof, wherein N^{A} is a nitrogen atom, and N^{A} is isotopically enriched with nitrogen-15.
28. A recombinant polynucleotide encoding a KabA polypeptide, a KabC polypeptide, or both.
29. The recombinant polynucleotide of embodiment 28, wherein the polynucleotide encodes both the KabA polypeptide and the KabC polypeptide.
30. The recombinant polynucleotide of embodiment 28 or 29, wherein the recombinant KabC polypeptide comprises an amino acid sequence that is at least 80% identical to any of SEQ ID NOs: 1-5.
31. The recombinant polynucleotide of any one of embodiments 28-30, wherein the recombinant KabC polypeptide is encoded by a polynucleotide that is at least 80% identical to any of SEQ ID NOs: 6-10.
32. The recombinant polynucleotide of any one of embodiments 28-31, wherein the recombinant KabA polypeptide comprises an amino acid sequence that is at least 80% identical to any of SEQ ID NOs: 11-15.
33. The recombinant polynucleotide of any one of embodiments 28-32, wherein the recombinant KabA polypeptide is encoded by a polynucleotide that is at least 80% identical to any of SEQ ID NOs: 16-20.
34. The recombinant polynucleotide of any one of embodiments 28-33, wherein the KabA polypeptide, the KabC polypeptide, or both is a Digenea spp. polypeptide, a Palmaria spp. polypeptide, a Rhodophysema spp. polypeptide, or a Grateloupia spp. polypeptide; optionally wherein the Digenea spp. is Digenea simplex, the Palmaria spp. is Palmaria palmata or Palmaria hecatensis, the Rhodophysema spp. is Rhodophysema elegans, and/or the Grateloupia spp. is Grateloupia filicina.
35. An expression vector comprising the recombinant polynucleotide of any one of embodiments 28-34.
36. A recombinant cell comprising a polynucleotide of any one of embodiments 28-34, or a vector of embodiment 35.
37. The recombinant cell of embodiment 36, wherein the cell is a microorganism.
38. The recombinant cell of embodiment 36, wherein the cell is a bacterium.
39. The recombinant cell of embodiment 36, wherein the cell is E. coli.
40. A process for preparing a compound having the formula,

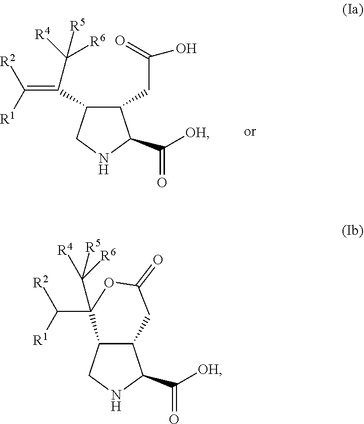

or a salt thereof, wherein the process comprises culturing a recombinant cell of any one of embodiments 36-39 in a culture medium.
41. The process of embodiment 40, wherein the culture medium comprises D-glucose.
42. The process of embodiment 40 or 41, further comprising isolating the compound of Formula Ia or Ib, or the salt thereof.

1. A process for preparing a compound having the formula, 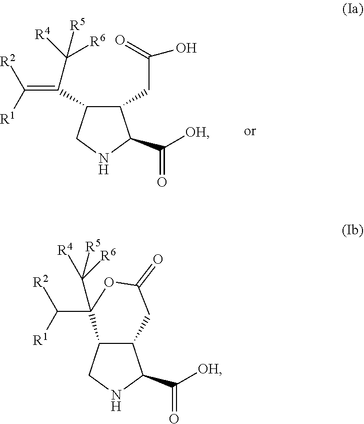
or a salt thereof, wherein the compound or a salt of Formula (Ia) or (Ib) is prepared by cyclizing a compound of formula II: 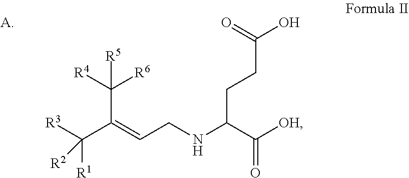
or a salt thereof; wherein the cyclization comprises contacting compound of Formula II with a recombinant KabC polypeptide; wherein:
(a) at least one of R¹, R², R³, R⁴, R⁵ or R⁶ is hydrogen; and
(b)
R¹ is hydrogen, halogen, —CN, —C(O)OH, —CONH₂, —NO₂, —SO₂Cl, —SO₂NH₂, —NHNH₂, —NHSO₂CH₃, —N₃, —NR^{1A}R^{1B}, —OR^{1A}, —SR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CN, —C(O)OH, —CONH₂, —NO₂, —SO₂Cl, —SO₂NH₂, —NHNH₂, —NHSO₂CH₃, —N₃, —NR^{2A}R^{2B}, —OR^{2A}, —SR^{2A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CN, —C(O)OH, —CONH₂, —NO₂, —SO₂Cl, —SO₂NH₂, —NHNH₂, —NHSO₂CH₃, —N₃, —NR^{3A}R^{3B}, —OR^{3A}, —SR^{3A} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CN, —C(O)OH, —CONH₂, —NO₂, —SO₂Cl, —SO₂NH₂, —NHNH₂, —NHSO₂CH₃, —N₃, —NR^{4A}R^{4B}, —OR^{4A}, —SR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CN, —C(O)OH, —CONH₂, —NO₂, —SO₂Cl, —SO₂NH₂, —NHNH₂, —NHSO₂CH₃, —N₃, —NR^{5A}R^{5B}, —OR^{5A}, —SR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CN, —C(O)OH, —CONH₂, —NO₂, —SO₂Cl, —SO₂NH₂, —NHNH₂, —NHSO₂CH₃, —N₃, —NR^{6A}R^{6B}, —OR^{6A}, —SR^{6A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
any two of R¹, R² or R³ substituents or any two of R⁴, R⁵ or R⁶ substituents may optionally be joined to form a substituted or unsubstituted aryl or substituted or unsubstituted heteroaryl;
any one of R¹, R² or R³ and any one of R⁴, R⁵ or R⁶ may optionally be joined to form a substituted or unsubstituted cycloalkyl, substituted or unsubstituted aryl or substituted or unsubstituted heteroaryl; and
each R^{1A}, R^{2A}, R^{3A}, R^{4A}, R^{5A}, R^{6A}, RIB R^{2B}, R^{3B}, R^{4B}, R^{5B}, and R^{6B} is independently a hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

2. The process of claim 1, wherein:
(a) at least one of R¹, R², R³, R⁴, R⁵ or R⁶ is hydrogen; and
(b)
R¹ is hydrogen, substituted or unsubstituted C₁-C₁₀ alkyl, substituted or unsubstituted C₅-C₁₀ aryl, or substituted or unsubstituted 5 to 10 membered heteroaryl;
R² is hydrogen, substituted or unsubstituted C₁-C₁₀ alkyl, substituted or unsubstituted C₅-C₁₀ aryl, or substituted or unsubstituted 5 to 10 membered heteroaryl;
R₃ is hydrogen, substituted or unsubstituted C₁-C₁₀ alkyl, substituted or unsubstituted C₅-C₁₀ aryl, or substituted or unsubstituted 5 to 10 membered heteroaryl;
R⁴ is hydrogen, substituted or unsubstituted C₁-C₁₀ alkyl, substituted or unsubstituted C₅-C₁₀ aryl, or substituted or unsubstituted 5 to 10 membered heteroaryl;
R⁵ is hydrogen, substituted or unsubstituted C₁-C₁₀ alkyl, substituted or unsubstituted C₅-C₁₀ aryl, or substituted or unsubstituted 5 to 10 membered heteroaryl;
R⁶ is hydrogen, substituted or unsubstituted C₁-C₁₀ alkyl, substituted or unsubstituted C₅-C₁₀ aryl, or substituted or unsubstituted 5 to 10 membered heteroaryl;
any two of R¹, R² or R³ substituents or any two of R⁴, R⁵ or R⁶ substituents may optionally be joined to form a substituted or unsubstituted C₅-C₁₀ aryl or substituted or unsubstituted 5 to 10 membered heteroaryl; and
any one of R¹, R² or R³ and any one of R⁴, R⁵ or R⁶ may optionally be joined to form a substituted or unsubstituted C₃-C₈ cycloalkyl, substituted or unsubstituted C₅-C₁₀ aryl or substituted or unsubstituted 5 to 10 membered heteroaryl.

3. (canceled)

4. (canceled)

5. The process of claim 1, wherein the process comprises contacting the compound of Formula II, or a salt thereof, with a purified KabC polypeptide and a α-keto-glutarate.

6. The process of claim 1, wherein the process for preparing the compound of Formula II, or a salt thereof, comprises:
(a) contacting a compound of Formula III: 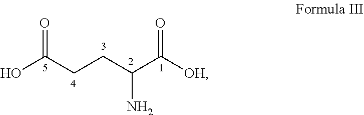
or a salt thereof, with a compound of Formula IV: 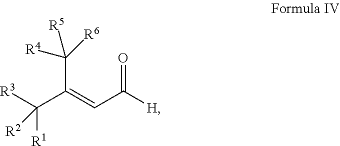
or a salt thereof, to form an imine, and
(b) reducing the imine to produce the compound of Formula II.

7. The process of claim 1, wherein the contacting comprises contacting the compound of Formula II, or a salt thereof, with a recombinant microorganism expressing the recombinant KabC polypeptide.

8. (canceled)

9. (canceled)

10. The process of claim 7, wherein the microorganism further expresses a recombinant KabA polypeptide.

11. (canceled)

12. (canceled)

13. The process of claim 7, wherein the recombinant microorganism is a bacterium.

14. (canceled)

15. A process for preparing a compound having the formula, 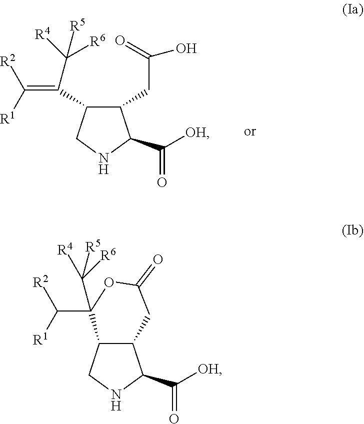
or a salt thereof, wherein the process comprises:
contacting a compound of Formula III: 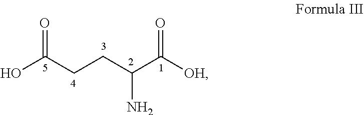
or a salt thereof, with a compound of Formula V: 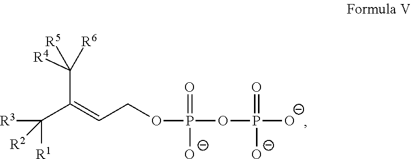
or a salt thereof, a recombinant KabA polypeptide, a recombinant KabC polypeptide, and an α-keto-glutarate.

16. The process of claim 15, wherein the recombinant KabA polypeptide and recombinant KabC polypeptide are each expressed by a recombinant microorganism.

17. (canceled)

18. (canceled)

19. (canceled)

20. (canceled)

21. The process of claim 15, wherein the recombinant KabA polypeptide and the recombinant KabC polypeptide are expressed by the same recombinant microorganism.

22. The process of claim 15, wherein the microorganism is a bacterium.

23. (canceled)

24. A compound of Formula II: 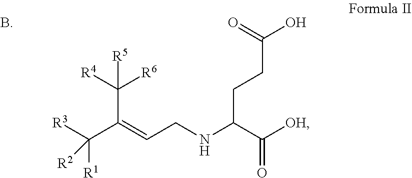
or a salt thereof.

25. A compound of Formula VI, VII, or VIII: 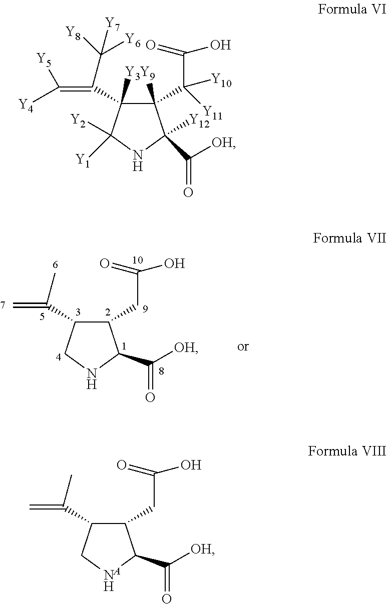
or a salt thereof,
wherein:
one of Y₁, Y₂, Y₃, Y₄, Y₅, Y₆, Y₇, Y₈, Y₉, Y₁₀, Y₁₁, or Y₁₂ is a hydrogen that is isotopically enriched with deuterium or tritium, and the rest of Y₁, Y₂, Y₃, Y₄, Y₅, Y₆, Y₇, Y₈, Y₉, Y₁₀, Y₁₁, and Y₁₂ are non-enriched hydrogen atoms;
1, 2, 3, 4, 5, 6, 7, 8, 9, and 10 are carbon atoms; and at least one of 1, 2, 3, 4, 5, 6, 7, 8, 9, or 10 carbon atom is isotopically enriched with carbon-13 or carbon-14; and
N^{A} is a nitrogen atom, and N^{A} is isotopically enriched with nitrogen-15.

26. (canceled)

27. (canceled)

28. A recombinant polynucleotide encoding a KabA polypeptide, a KabC polypeptide, or both.

29. (canceled)

30. (canceled)

31. (canceled)

32. (canceled)

33. (canceled)

34. The recombinant polynucleotide of claim 28, wherein the KabA polypeptide, the KabC polypeptide, or both is a Digenea spp. polypeptide, a Palmaria spp. polypeptide, a Rhodophysema spp. polypeptide, or a Grateloupia spp. polypeptide; optionally wherein the Digenea spp. is Digenea simplex, the Palmaria spp. is Palmaria palmata or Palmaria hecatensis, the Rhodophysema spp. is Rhodophysema elegans, and/or the Grateloupia spp. is Grateloupia filicina.

35. An expression vector comprising the recombinant polynucleotide of claim 28.

36. A recombinant cell comprising a polynucleotide of claim 28.

37. The recombinant cell of claim 36, wherein the cell is a microorganism.

38. The recombinant cell of claim 36, wherein the cell is a bacterium.

39. (canceled)

40. A process for preparing a compound having the formula, 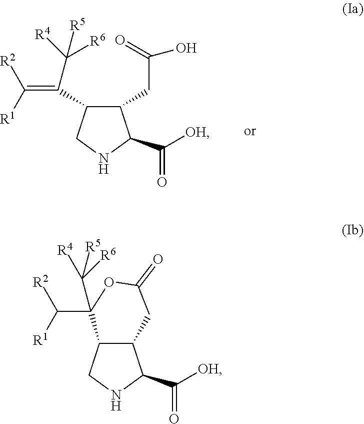
or a salt thereof, wherein the process comprises culturing a recombinant cell of claim 36 in a culture medium.

41. (canceled)

42. (canceled)
